(19) United States
(12) Patent Application Publication
(10) Pub. No.: US 2019/0256813 A1
(43) Pub. Date: Aug. 22, 2019

(54) CELL CULTURE DISH SUITABLE FOR IN SITU ELECTROPORATION AND INDUCING DESIRED CELL POTENCY AND OTHER BEHAVIORS

(71) Applicant: Christopher B. REID, Los Angeles, CA (US)

(72) Inventor: Christopher B. REID, Los Angeles, CA (US)

(21) Appl. No.: 16/171,420

(22) Filed: Oct. 26, 2018

Related U.S. Application Data
(63) Continuation-in-part of application No. 14/764,195, filed on Jul. 29, 2015, now Pat. No. 10,138,451.
Application No. 14/764,195 is a continuation-in-part of application No. 12/601,819, filed on May 31, 2011.

(51) Int. Cl. C12M 3/06; C12M 1/00; C12N 5/00; C12M 1/42; C12N 13/00

(52) U.S. Cl. CPC C12M 23/16; C12M 23/34; C12N 13/00; C12M 35/02; C12N 5/0062

(57) ABSTRACT

A cell culture dish made of non media-permeable material and having a base and a plurality of separate juxtaposed side-by-side wells having common interior well walls preventing physical contact or movement of chemical factors between the separated cell or tissue cultures, the walls having different heights and defining an outer “surround” cell or tissue culture and one or more inner “center” cell or tissue cultures to enable contained cell or tissue communication between the well spaces, the wells configured to comprise two or more cell or tissue cultures, the wells permitting signal communication between the cells or tissues situated within said wells, and further comprising wall material, electrode contacts electrodes and/or electrode contacts, and well dimensions suitable for facilitating electroporation. The inventor observed 100% efficiency in cell reprogramming with protein electroporation-orders of magnitude above other reported methods.

### BACKGROUND OF THE INVENTION

The present invention relates to a novel culturing apparatus (assembly) which can be termed a cell culture dish. More particularly, the present invention is a “combined cell culture dish” or “dish-in-dish” apparatus comprising at least one smaller cell culture dish fixedly positioned within a larger cell culture dish, and the number of such fixated cell culture dishes can include a multiple number of fixated cell culture dishes within one another, either concentric or eccentric, in any number of geometric shapes, and without limitation to the number of petri dishes included. An alternate embodiment of this invention can include a plurality of cell culture dishes juxtaposed side-by-side having common interior well walls, and the well walls may or may not be different in height depending on the application. The combined cell culture dish differs from the prior art because the walls of said combined petri dishes may be of different heights and made from any combination of transparent and non-transparent materials that will allow juxtaposing and different cultures to grow simultaneously. Such separate but juxtaposing culture growth can then be studied to determine whether certain cultures grown separately and in close proximity influence each other in certain ways. The combined cell culture dish of the present invention may or may not be fitted with single or multiple covers and may or may not be stacked.

Examples of prior petri dishes may be found in the following U.S. Pat. No. 4,675,298 (Brusewitz, Gerhard). U.S. Pat. No. 4,160,700 (Boomus, Mary), and U.S. Pat. No. 3,660,243 (Young, Cecil).

The transcendent challenge for medicine in the 21st century will be replacing damaged, worn-out or genetically-compromised cells. Transcription factors binding specifically to DNA play a vital role in regulating gene expression. It is the particular complement of transcription factors within an individual cell, that determine which cellular programs are active and which are turned off. In this capacity transcription factors play a decisive role in determining and maintaining cellular identity, as well as determining cellular vulnerability.

### SUMMARY OF THE INVENTION

The present invention is directed to a novel cell culture dish having a multi-chambered construction which facilitates juxtaposition of different physically separated cultures. Said novel multi-chambered cell culture dish will permit co-culturing of any two or more separate cultures, whether those cultures are species related or not.

In general, the cell culture dish of the present invention comprises two or more dishes which create a central compartment and one or more peripheral compartments which surround the central compartment. Said central and peripheral compartments may take the form of any shape, including, but not limited to. cylindrical, square, pentagonal, or hexagonal. The material used to construct said petri dish may include, but may not be limited to. any non media-permeable form of glass, plastic or metal or combination thereof, which will sustain culture growth and permit observation and recording of said culture growth, including, but not limited to. the recording of signal transduction. Separated areas created by utilizing the central compartment and one or more peripheral compartments may be geometrically concentric or eccentric.

The petri dish of the present invention may comprise one or more dishes within a dish or may be constructed of a single dish with a flat well bottom having one or more sets of walls that extend from said well bottom forming one or more separate enclosures having the same geometric shape or a variety of geometric shapes. The wall or walls are constructed to ensure physical isolation of two or more sets of cells from one another to prevent physical contact between the separated cells or movement of chemical factors originating in the media or within the cells. The separate wells may or may not be numbered to enhance the identification of certain cell cultures.

A preferred embodiment of this invention is depicted in FIG. 1 below.

The petri dish described above can be sterilized using either wet or dry heat. However, the petri dish may be a single use device as well. The outer wall of the multi-chambered petri dish can be sized appropriately to fit high-perform an c e incubation and perfusion chambers for live cell imaging and to withstand temperatures ranging from 5 degrees below Celsius to 50 degrees above Celsius. However, the multi-chambered petri dishes of this invention may also withstand a host of temperatures outside the previously stated range.

One object of the present invention is to provide a multi-chambered cell or tissue culture dish suitable for assessing cell communication that is not prohibited by intervention of the chamber walls.

Another object of this invention is to provide a cell or tissue culture dish having a plurality of separate wells which permit communication between cells or tissues situated within said wells of signals or communication which might emanate from said cells or tissues.

Still another object of this invention is to provide a multi-chambered cell or tissue culture dish with a transparent and flat bottom to enable convenient and accurate viewing and analysis of the contents of each separate chamber.

A further object of this invention is to provide a multi-chambered cell or tissue culture dish which can be made from a number of transparent materials, including, but not limited to. glass, acrylic polymers, fluorinated ethylene propylene, ultra high molecular weight polyethylene, polycarbonate, polystyrene, or any amorphous high-performance polymer, with or without electrodes and/or electrode contacts facilitating electroporation.

Yet another object of this invention is to provide a multi-chambered cell or tissue culture dish manufactured with well walls having different heights and defining an outer “surround” cell culture and one or more inner “center” cell cultures to enable contained cell or tissue communication within the well spaces. Such communication would include, but would not be limited to. putative nociceptive cell signaling in physically disconnected but proximal cell populations, including cell-to-cell communications which are taking place after eliminating the availability of any potential pathways for neural or diffusible factor mediated cell-cell communication.

The ability to derive proliferating, self-renewing, multipotent and pluripotent cell population(s) from otherwise non-pluripotent, non-self renewing cells may have significant positive implications for all fields utilizing cellular therapies. These fields include bone marrow transplantation, transfusion medicine, and gene therapy and enable the production of patient-specific stem cells and other desired cell types. Likewise, the ability to initiate differentiation of cells into neural, muscle, and various other desirable stem and somatic cell populations is and will also be of significant value to medicine and commercial processes involving animals. Accordingly, the present invention provides methods for genetic production and uses of self-renewing cell populations, totipotent cell populations, multipotent cell populations, pluripotent cell populations, and differentiating/differentiated cell populations, e.g. neuronal cell populations, muscle cell populations, hematopoietic cell populations, etc., and other desired cell populations such as, for example, HIV resistant cell populations.

It is a proposition of the present invention that the efficient introduction or overexpression of specific transcription factors, alone or in combination with other cell fate determinants (e.g. notch, numb, numblike and other proteins, as well as certain miRNAs and other non-coding RNAs), enables the interconversion of what have been considered transitory (multipotent, pluripotent, and/or self-renewing) or fixed (differentiated or somatic) cellular phenotypes. The ability to reliably induce phenotypic conversion or cellular reprogramming allows the production of stem cells, replacement cells, tissues, and organs that match individual patients. In conjunction with gene therapy techniques and cell culture techniques, cell type interconversion also provides for the production of disease-resistant and genetically-repaired cells that are suitable for transplantation.

It is an object of this invention to provide various manners of generating proliferating, self-renewing, totipotent, multipotent and/or pluripotent cell population(s), as well as other desirable differentiating/differentiated cell populations, from either dividing or non-dividing cells without the use of oncogenes. Differentiating cell populations comprise cells expressing some, but not all markers associated with specific cell type categorization. It is disclosed herein, for example, that appropriate Numb isoform expression in combination with other transgenes/proteins (especially transcription factors) enables the production of dividing, pluripotent cell populations or differentiating cell populations. Moreover, the genetic vectors of the present invention may be used to produce genetic modification (e.g. expression of gene products deficient in the patient) and to transiently or permanently induce proliferation, self-renewal, or stem/progenitor cell behavior in endogenous cells in vivo, particularly those cells found in tissues which normally do not show or no longer show such behavior. Finally, other genetic vectors of the present invention may be used to produce genetic modification and/or to block proliferation, self-renewal, or stem/progenitor cell behavior in cells aberrantly displaying such behavior (e.g. cancer cells). It is also an object of the present invention to provide therapeutic vectors and cells capable of expressing synthetic oligonucleotide sequences predicted to attenuate disease processes. For example, the current invention discloses the use of synthetic oligonucleotides to reduce gene expression critical HIV and other immunodeficiency virus infection, propagation and spread.

The invention may be used with any suitable cells, including vertebrate cells, and including fish, mammalian, avian, amphibian, and reptilian cells.

The inventor observed 100% efficiency in cell reprogramming with protein electroporation-orders of magnitude above other reported methods.

### BRIEF DESCRIPTION OF FIGURES

FIG. 1 is a front view of a two well cell or tissue culture dish constructed according to this invention, containing different cell or tissue cultures within each separate well.

FIG. 2 is a front view of a two well cell or tissue culture dish constructed according to this invention with the wells positioned side-by-side.

FIG. 3 is a front view of a three well cell or tissue culture dish constructed according to this invention with the wells positioned side-by-side.

FIG. 4 is a front view of a nine well cell or tissue culture dish constructed according to this invention with the wells positioned side-by-side.

FIG. 5 is a schematized vector map corresponding to the vector sequence of Example 13.

### DETAILED DESCRIPTION OF FIGURES

The multi-chambered cell or tissue culture dish shown in FIG. 1 is composed of a common base 1 made of the same transparent chemical resistant material. In the present embodiment, two wells are formed as depicted, the center well being defined by well wall 3 and the surround well being defined by well wall 2 which is dimensionally higher, as can be determined by measuring from the base 1 to the surround cell wall rim 4 than is well wall 3 which is measured from the base 1 to the center cell wall rim 5. It is understood that a greater number of center wells may be provided depending on the application having differing cell wall heights. FIG. 1 also depicts a cell or tissue culture 7, situated in the surround well, which is defined dimensionally by the base 1, the surround well wall 2 and the center well wall 3. FIG. 1 also depicts a cell or tissue culture 6, situated in the center well as defined dimensionally by the base 1, and the center well wall 3.

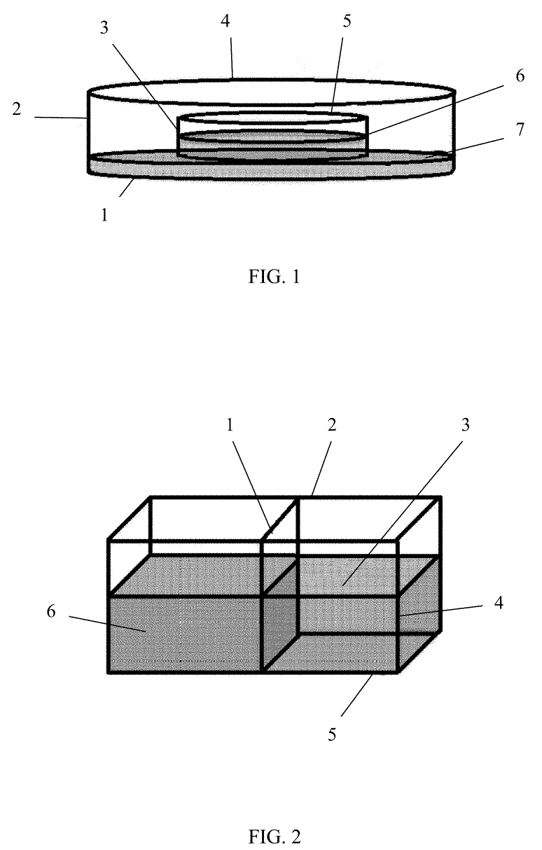

The multi-chambered cell or tissue culture dish shown in FIG. 2 is composed of a common base 5 made of the same transparent chemical resistant material. In the present embodiment, two wells are formed as depicted, the left well being separated from the right well by well wall 1 and the right well being defined by well walls 1, 2, 3, and 4. It is understood that a greater number of wells than the two depicted may be juxtaposed together depending on the application. FIG. 2 also depicts a cell or tissue culture 6 situated in the left well.

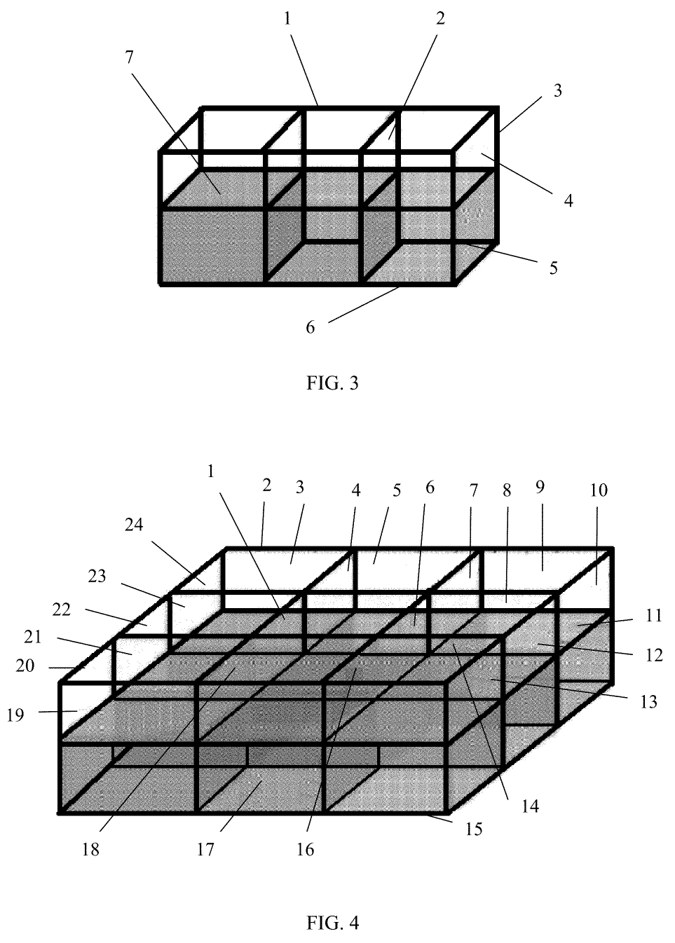

The multi-chambered cell or tissue culture dish shown in FIG. 3 is composed of a common base 6 made of the same transparent chemical resistant material. In the present embodiment, three wells are formed as depicted, the right well being separated from the middle well by well wall 2 and the right well being defined by well walls 2, 3, 4, and 5. It is understood that a greater number of wells than the three depicted may be juxtaposed together depending on the application. FIG. 3 also depicts a cell or tissue culture 7 situated in the far left well.

The multi-chambered cell or tissue culture dish shown in FIG. 4 is composed of a common base 15 made of the same transparent chemical resistant material as the remainder of well walls. In the present embodiment, nine wells are formed as depicted, the right well being separated from the middle well by well wall 2 and the first well being defined by well walls 24, 3, 4, and 1. the second well being defined by well walls 4, 5, 6, and 7, the third well being defined by well walls 7, 8, 9, and 10, the fourth well being defined by well walls 6, 8, 12, and 14, the fifth well being defined by well walls 14, 14, 16, and a front wall, the sixth well being defined by well walls 16, 17, 18 and a rear wall, the seventh well being defined by well walls 18, 19, 20 and 21, the eighth well being defined by well walls 21, 22, 23 and an interior well wall, and the ninth well being defined as the well situated in the center and surrounded by wells 1 through 8, sharing common walls with those wells. It is understood that a greater number of wells than the nine depicted may be juxtaposed together depending on the application. FIG. 4 also depicts a cell or tissue culture 13 situated in the fourth well.

It will be clear to a person skilled in the art that specific embodiments discussed herein are not the only possible modes of this invention that can be manufactured. Many other features that are not shown in the described embodiments are within the scope of this invention.

### DETAILED DESCRIPTION

All patents, patent applications, and publications cited in this application are hereby incorporated by reference herein in their entireties.

As discussed herein, ‘“DNA” refers to deoxyribonucleic acid and “RNA” refers to ribonucleic acid. As discussed herein, “cDNA” refers to complementary DNA; “mRNA” refers to messenger RNA; “siRNA” refers to small interfering RNA; “shRNA” refers to small hairpin RNA; “miRNA’” refers to microRNA, such as single-stranded RNA molecules, typically about 20-30 nucleotides in length, which may regulate gene expression; “decoy” and “decoy RNA” and “RNA decoy” refer to an RNA molecule that mimics the natural binding domain for a ligand.

As used herein, the meaning of the term “ameliorating” includes lessening an effect, or reducing damage, or minimizing the effect or impact of an action, activity, or function, and includes, for example, lessening the deleterious effects of a disease or condition.

As used herein, the meaning of the term “retarding” includes slowing or lessening the progress of an effect or action, and includes, for example, slowing the progress of a disease, slowing the rate of infection, or otherwise acting to slow or reduce the advance or progress of a disease or condition.

As used herein, an “inducing agent” is an agent that aids or is alone effective to promote an action. For example, an exogenous agent that affects a promoter, e.g., by initiating or enhancing its activity, and so affects expression of a gene under control of the promoter, may be termed an inducing agent. For example, tetracycline may be used as an inducing agent; and doxycycline may be used as an inducing agent.

A nucleic acid sequence (e.g., a nucleic acid sequence encoding a polypeptide) is termed “operably linked” to another nucleic acid sequence (e.g., a promoter) when the first nucleic acid sequence is placed in a functional relationship with the second nuceleic acid sequence. For example, a promoter is operably linked to a coding sequence if the promoter affects the transcription or expression of the coding sequence. As used herein, the term “driven by” refers to a gene or coding sequence that is operably linked to a promoter sequence, and that the promoter sequence affects the transcription or expression of the coding sequence.

As used herein, a “marker” is a molecule that is detectable, or codes for a detectable molecule, or acts on other molecules so that the presence of the marker is detectable. A “marker protein” or “marker polypeptide” is a protein or polypeptide that is detectable in a laboratory or clinical environment, and, in embodiments, may be detectable by eye. A “marker gene” encodes a marker protein or marker polypeptide.

As used herein, “HIV” refers to human immunodeficiency virus, and includes variants such as, e.g., HIV-I, HIV-2. Other immunodeficiency viruses include simian immunodeficiency virus (SIV) and feline immunodeficiency virus (FIV). Enzymes related to IHV may be termed “HIV enzymes” and include, for example,\integrase, protease, reverse transcriptase, and transactivating regulatory protein (TAT).

Infection by HIV is believed to involve receptors termed “HIV receptors.” There may be multiple such receptors, some of which may be termed “HIV co-receptors.” As discussed herein, HIV co-receptors include CXCR4 and CCR5.

A theoretical basis for the embodiments of the invention is described herein, however, this discussion is not in any way to be considered as binding or limiting on the present invention. Those of skill in the art will understand that the various embodiments of the invention may be practiced regardless of the model used to describe the theoretical underpinnings of the invention.

In a preferred embodiment, cells are “selected” from accessible, dividing or non-dividing cell populations for the purpose of generating the desired a) proliferating, multipotent or pluripotent cell population, differentiating b) populations of neuronal cells c) muscle cells, d) and/or any other desired cell population; moreover the desired cell population may be capable of further differentiation in vitro, in vivo, and/or tissue-appropriate and regionally-appropriate differentiation in vivo.

Sources of cells selected for use in the invention:

Selected cells may include any cell practicable in the present invention. Cells selected for use in the present invention (herein termed “selected cells”) may originate as endogenous cells of the patient—including cells derived from other organ systems; or from exogenous sources (including those derived from cell lines, cryopreserved sources, banked sources, and donors). Cells may also be selected from cells genetically-modified with synthetic or natural nucleic acid sequences. The term “selected cells” as used herein does not include human embryonic stem cells.

In embodiments of the present invention, in order that they may be isolated without the involvement of invasive procedures, selected cells will preferably be easily accessible cells (e.g. peripheral blood leukocytes, circulating hematopoietic stem cells, epithelial cells (e.g. buccal cheek cells (e.g. Michalczyk et al, 2004)), adipose tissue (e.g. Gimble et al., 2007; Ma et al., 2007), umbilical cord blood cells (e.g. Zhao, et al., 2006; Tian et al., 2007), etc.). However, bone marrow stem cells, spermatogonia (e.g. Guan el al., 2006; Takahashi et al., 2007), primordial germ cells (PGCs), stem cells isolated from amniotic membranes (e.g. Ilancheran et al., 2007), amniotic fluid (e.g. De Coppi et al., 2007), as well as cells isolated from the skin (e.g. Tumbar, 2006; Dunnwald et al., 2001; Szudal'tseva et al., 2007), etc., are also covered by the present invention. Such cells can be isolated from the tissues in which they reside by any means known to the art.

Spermatogonia cells can be isolated using a two-step enzymatic digestion followed by Percoll separation. Cells can then be resuspended in minimum essential medium (MEM) supplemented with bovine serum albumin to a final concentration of 106/mL. In detail: Tubule fragments are accessed surgically and teased apart prior to treatment with 1 mg/ml trypsin, hyaluronidase, and collagenase, and then 1 mg/ml hyaluronidase and collagenase, in MEM containing 0.10% sodium bicarbonate, 4 mM L-glutamine, nonessential amino acids, 40 microgram/ml gentamycin, 100 KJ to 100 microgram/ml penicillin-streptomycin, and 15 mM HEPES. Spermatogonia cells are further separated from tubule fragments by centrifugation at 30 times gravity. After filtration through nylon filters with 77- and/or 55-micron pore sizes, cells are collected and loaded onto a discontinuous Percoll density gradient. Fractions with a purity greater than 40% progenitor/stem/spermatogonia cells are washed and resuspended to a concentration of cells equivalent to 10 progenitor/stem/spermatogonia cells per ml. Afterwards cells are cultured and/or stored by any cryopreservation technique known to the art.

The selected cells may be genetically-modified cells, especially cells that have been genetically modified by any means known to the art, to encode therapeutic or commercially useful deoxyribonucleic acid (DNA) or ribonucleic acid (RNA) sequences.

In accordance with an aspect of the present invention, there is provided a method of producing a desired cell population (e.g. pluripotent, neuronal, muscle, etc.) from the selected cells.

Achieving multipotent, pluripotent, and/or self renewing cell populations:

In order to achieve a) a population of proliferating, self renewing pluripotent cells, the selected cell(s) and/or their progeny are transfected/contacted with nucleotide sequence(s) including those encoding the “long” (PRR insert+) isoform(s) of the mammalian numb gene. At about the same time the selected cells may also be transfected/contacted with synthetic oligonucleotides targeting the short Numb isoforms and Numblike, then cultured under conditions which promote growth of the selected cells at an optimal growth rate. Selected cells are maintained under these conditions for the period of time sufficient to achieve the desired cell number.

The cells are grown at the (optimal) rate of growth achieved by incubation with LIF, steel factor, and/or equipotent concentrations of 11-6, hyper 1L-6, IL-7, oncostatin-M and/or cardiotrophin-1; or that growth rate achieved in the presence of other growth enhancing cytokines (e.g. those conditions described for culturing pluripotent cells e.g. Guan et al., 2006). The growth rate is determined from the doubling times of the selected cells in said growth culture medium. Likewise, culture conditions such as those described in U.S. Pat. Nos. 6,432,711 and 5,453,357 may also be suitable for the propagation and expansion, at an optimal growth rate, of cells transfected/contacted with the long (PRR+) Numb isoform(s). Other appropriate protocols and reference cytokine concentrations have been taught by Koshimizu et al., 1996; Keller et al., 1996; Piquet-Pellorce, 1994; Rose et al., 1994; Park and Han, 2000; Guan et al., 2006; Dykstra et al., 2006; Zhang et al., 2007). However the practice of the present invention is not limited to the details of these teachings.

In a preferred embodiment, the selected cells are cultured in a standard growth medium (e.g. Minimal Essential Medium with or without supplements (e.g. glutamine, and beta.-mercaptoethanol). The medium may include basic fibroblast growth factor (bFGF), steel factor, leukemia inhibitory factor (LIF), and/or factors with LIF activity (e.g. LIF, LIF receptor (LIFR), ciliary Neurotrophic factor (CNTF), oncostatin M (OSM), OSM receptor (OSMR), cardiotrophin, interleukins (IL) such as IL-6, hyper IL-6, GP 130, etc.) as well as horse serum. LIF, as well as other factors with LIF activity, prevents spontaneous differentiation of the cells. Under these conditions, selected cells transfected/contacted with the PRR+Numb isoform(s) and their progeny are expected to achieve multipotency, pluripotency and/or self-renewal.

In a preferred embodiment, the selected cell(s) and/or their progeny are transfected/contacted with nucleotide sequence(s) encoding the “long” (PRR insert+) Numb isoform(s) as well as sequences encoding other transgenes. Many of those transgenes are listed below along with their corresponding identification numbers (accession numbers) in the NCBI sequence database.

In another preferred embodiment, the selected cell(s) and/or their progeny are transfected with nucleotide sequence(s) encoding a portion of the “long” (PRR insert+) Numb isoform(s) as well as sequences encoding other transgenes. Many of those transgenes are listed below along with their corresponding identification (accession) numbers (codes) in the NCBI sequence database.

In a preferred embodiment, the selected cells and/or their progeny are transfected/contacted with long (PRR+) Numb isoform encoding sequences as well as sequences encoding other transgenes, including LIF.

In a preferred embodiment, the selected cells and/or their progeny are transfected/contacted with long (PRR+) Numb isoform encoding sequences as well as sequences encoding other transgenes, including ones with LIF activity.

In a preferred embodiment, the selected cells and/or their progeny are transfected/contacted with long (PRR+) Numb isoform(s) encoding sequences as well as sequences encoding other transgenes, including the LIFR.

In a preferred embodiment, the selected cells and/or their progeny are transfected/contacted with long (P RR+) Numb isoform(s) encoding sequences as well as sequences encoding other transgenes, including oncostatin M (OSM).

In a preferred embodiment, the selected cells and/or their progeny are transfected/contacted with long (PRR+) Numb isoform(s) encoding sequences as well as sequences encoding other transgenes, including oncostatin M receptor (OSMR).

In a preferred embodiment, the selected cells and/or their progeny are transfected/contacted with long (PRR+) Numb isoform(s) encoding sequences as well as sequences encoding other transgenes, including cardiotrophin-1.

In a preferred embodiment, the selected cells and/or their progeny are transfected/contacted with long (PRR+) Numb isoform(s) encoding sequences as well as sequences encoding other transgenes, including CNTF.

In a preferred embodiment, the selected cells and/or their progeny are transfected/contacted with long (PRR+) Numb isoform(s) encoding sequences as well as sequences encoding other transgenes, including OCT3/4 and SOX2.

In a preferred embodiment, the selected cells and/or their progeny are transfected/contacted with long (PRRf) Numb isoform(s) encoding sequences as well as sequences encoding other transgenes, including NANOG, OCT3/4 and SOX2.

In a preferred embodiment, the selected cells and/or their progeny are transfected/contacted with long (PRR+) Numb isoform(s) encoding sequences as well as sequences encoding other transgenes, including OCT3/4 and SOX2 and a transgene with LIF activity.

In a preferred embodiment, the selected cells and/or their progeny are transfected/contacted sequences encoding other transgenes, including OCT3/4 and SOX2 and a transgene with LIF activity.

In a preferred embodiment, the selected cells and/or their progeny are transfected/contacted with long (PRR+) Numb isoform(s) encoding sequences as well as sequences encoding other transgenes, including Notch (e.g. Gaiano et al., 2000).

In a preferred embodiment, the selected cells and/or their progeny are transfected/contacted with long (PRR+) Numb isoform(s) encoding sequences as well as sequences encoding other transgenes, including OCT3/4, SOX2 and Notch (e.g. notch 1 and/or notch 2).

In a preferred embodiment, the selected cells and/or their progeny are transfected/contacted with long (PRR+) Numb isoform(s) encoding sequences as well as sequences encoding other transgenes, including OCT3/4, SOX2, NANOG, and Notch.

In a preferred embodiment, the selected cells and/or their progeny are transfected/contacted with long (P RR+) Numb isoform(s) encoding sequences as well as sequences encoding other transgenes, including OCT3/4, SOX2, NANOG, and a transgene with LIF activity.

In a preferred embodiment, the selected cells and/or their progeny are transfected/contacted with long (PRR+) Numb isoform(s) encoding sequences as well as sequences encoding other transgenes, including OCT3/4, SOX2, NANOG, and multiple transgenes with LIF activity.

In a preferred embodiment, the selected cells and/or their progeny are transfected/contacted with long (PRR+) Numb isoform(s) encoding sequences as well as sequences encoding other transgenes, including OCT3/4, Notch, HOXB4 and SOX2.

Over time, other gene combinations differing from those described herein may be described or discovered capable of causing cells to become multipotent, pluripotent, capable of self-renewal, or to begin differentiating. However this patent application covers such “‘genetic reprogramming” of any nucleated cell utilizing nucleic acid or protein electroporation (see Gagne et al., 1991; Saito et al., 2001; Yuan, 2008; Huang et al., 2007; Xia and Zhang, 2007; Cemazar and Sersa 2007; lsaka and Imai, 2007; Luxembourg et al., 2007; Van Tendeloos, 2007; Takahashi, 2007; etc.), liposomes, nanocapsules, nanovaults, etc. (see Goldberg et al., 2007; Li et al., 2007), and/or another approach avoiding viral integration or other random alteration of the cell's genome, as such means increase safety and efficiency.

Excluded, of course, from the category of random alteration are approaches involving gene-targeting and site-directed methods designed to introduce or remove DNA at specific locations in the genome.

Likewise, this patent application covers the genetic reprogramming of any nucleated cell utilizing nucleic acid or protein electroporation, liposomes, nanocapsules, nanovaults, etc., and/or another approach avoiding retroviral/lenti viral integration or other random alteration of the cell's genome, as such means increase safety and efficiency. Such approaches and methods include all known to the art and practicable in the present invention.

In a preferred embodiment, nucleic acid(s) or protein(s) corresponding to a single gene, or portion thereof, (particularly those named herein, discovered according to methods described herein, discovered according to other published methods; or known to be multipotency, pluripotency, or self-renewal inducing) are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to a single gene, or portion thereof, (particularly those named herein, discovered according to methods described herein, discovered according to other published methods; or known to be multipotency, pluripotency, or self-renewal inducing) are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; or known to be multipotency, pluripotency, or self-renewal inducing) so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; or known to be multipotency, pluripotency, or self-renewal inducing) so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to Nanog are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Nanog so long as a population of multipotent, pluripotent, and/or self-renewing cells is produced from the selected cells and the method utilized is electroporation, liposomes, nanocapsules, nanovaults, and/or another approach avoiding viral integration or other random alteration of the cell's genome.

In a separate preferred embodiment, nucleic acid(s) or protein(s) corresponding to Oct4 and Sox2 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding viral integration or other random alteration of the cell's genome.

In a separate preferred embodiment, other nucleic acid(s) or protein(s) are utilized in concert with the nucleic acid(s) or protein(s) corresponding to Oct4/Sox2 so long as a population of multipotent, pluripotent, and/or self-renewing cells is produced from the selected cells and the method utilized is electroporation, liposomes, nanocapsules, nanovaults, and/or another approach avoiding viral integration or other random alteration of the cell's genome.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding viral integration or other random alteration of the cell's genome.

In a separate preferred embodiment, other nucleic acid(s) or protein(s) are utilized in concert with the nucleic acid(s) or protein(s) corresponding to Long (PRR+) Numb isoforms so long as a population of multipotent, pluripotent, and/or self-renewing cells is produced from the selected cells and the method is electroporation, liposomes, nanocapsules, nanovaults, and/or another approach avoiding viral integration or other random alteration of the cell's genome.

In a preferred embodiment, nucleic acid(s) or protein(s) corresponding to Nanog are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to Nanog are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Nanog so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to Nanog so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to a gene with LIF activity are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to a gene with LIF activity are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to to a gene with LIF activity so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to a gene with LIF activity so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to Oct4 are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to Oct4 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding re troviral/lenti viral integration or other random alteration of the cell's genome.

In a separate preferred embodiment, other nucleic acid(s) or protein(s) can be utilized in concert with the nucleic acid(s) or protein(s) corresponding to Oct4 so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to Oct4 so long as a population of multipotent, pluripotent, and/or self-renewing cells is produced from the selected cells and the method utilized is electroporation, liposomes, nanocapsules, nanovaults, and/or another approach avoiding retroviral/lenti viral integration or other random alteration of the cell's genome.

In a preferred embodiment, nucleic acid(s) or protein(s) corresponding to Sox2 are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to Sox2 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Sox2 so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to Sox2 so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to Iin28 are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to Iin28 are the only nucleic acid(s) or protein(s) overexpressed and/or introduced to produce multipotent, pluripotent, and/or self-renewing cells from the selected cells and the method utilized is electroporation, liposomes, nanocapsules, nanovaults. and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to Iin28 so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a preferred embodiment, nucleic acid(s) or protein(s) corresponding to c-myc are the only nucleic acid(s) or protein(s) overexpressed and/or introduced to produce multipotent, pluripotent, and/or self-renewing cells from the selected cells.

In a preferred embodiment, nucleic acid(s) or protein(s) corresponding to c-myc are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to c-myc so long as a population of multipotent, pluripotent, and/or self-renewing cells is produced from the selected cells.

In a separate preferred embodiment, other nucleic acid(s) or protein(s) can be utilized in concert with the nucleic acid(s) or protein(s) corresponding to c-myc so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding Oct4 and Sox2 are the only nucleic acid(s) or protein(s) overexpressed and/or introduced to produce multipotent, pluripotent, and/or self-renewing cells from the selected cells.

In a separate preferred embodiment, nucleic acid(s) or protein(s) corresponding to Oct4 and Sox2 are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Oct4 and Sox2 so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Oct4 and Sox2 so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent, and/or self-renewing cells from the selected cells.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Long (PRR+) Numb Isoforms so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Long (PRR+) Numb Isoforms so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding to Oct4, Sox2, and Nanog are the only nucleic acid(s) or protein(s) overexpressed and/or introduced to produce multipotent, pluripotent, and/or self-renewing cells from the selected cells.

In a separate preferred embodiment, nucleic acid(s) or protein(s) corresponding to Oct4, Sox2, and Nanog are the only nucleic acid(s) or protein(s) overexpressed and/or introduced to produce multipotent, pluripotent, and/or self-renewing cells from the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Oct4, Sox2, and Nanog so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Oct4, Sox2, and Nanog so long as a population of multipotent, pluripotent, and/or self-renewing cells is produced from the selected cells and the method utilized is electroporation, liposomes, nanocapsules, nanovaults, and/or another approach avoiding retroviral/lentiviral integration or other random alteration of the cell's genome.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent. and/or self-renewing cells from the selected cells.

In a separate preferred embodiment, nucleic acid(s) or protein(s) corresponding to Long (PRR+) Numb isoforms are the only nucleic acid(s) or protein(s) overexpressed and/or introduced to produce multipotent, pluripotent, and/or self-renewing cells from the selected cells and the method is electroporation, liposomes, nanocapsules, nanovaults, and/or another approach avoiding retroviral/lentiviral integration or other random alteration of the cell's genome.

In a separate preferred embodiment, other nucleic acid(s) or protein(s) are utilized in concert with the nucleic acid(s) or protein(s) corresponding to Long (PRR+) Numb isoforms so long as a population of multipotent, pluripotent, and/or self-renewing cells is produced from the selected cells.

In a separate preferred embodiment, other nucleic acid(s) or protein(s) are utilized in concert with the nucleic acid(s) or protein(s) corresponding to Long (PRR+) Numb isoforms so long as a population of multipotent, pluripotent, and/or self-renewing cells is produced from the selected cells and the method is electroporation, liposomes, nanocapsules, nanovaults, and/or another approach avoiding retroviral/lentiviral integration or other random alteration of the cell's genome.

It is to be understood that any combination of nucleic acid or protein sequences described herein can be modified by excluding those corresponding to Numb and/or Numblike so long as the desired cell population or behavior is achieved.

Similarly, it should be understood that the methods described herein for initiating differentiation are applicable to any induced or non-induced multipotent, pluripotent, or self-renewing stem cells, other progenitor cells, or other selected cells, not only those obtained in the manner described herein.

It is to be understood that any combination of nucleic acid or protein sequences described herein can be modified by excluding nucleic acid sequences or proteins corresponding to Numb and/or Numblike so long as the desired cell population is achieved.

In another embodiment, the various nucleic acid or protein combinations described herein are employed with the exclusion of the nucleic acid or protein corresponding to the Numblike and/or Numb isoforms.

In a preferred embodiment, the selected cells and/or their progeny are cells that have been genetically-modified beforehand.

In a preferred embodiment, the transfection/contacting steps described herein represent transient transfection.

In a further preferred embodiment such transient transfection is accomplished using viral vectors that do not integrate into the host genome.

In another preferred embodiment, such transient transfection is accomplished using standard transfection techniques (electroporation, chemically mediated transfection, fusogenic or non-fusogenic liposomes, nanocapsules, nanovaults, etc.).

Over time, other gene combinations differing from those described herein may be described or discovered capable of causing cells to become multipotent, pluripotent, capable of self-renewal or to begin differentiating. However this patent application also covers the genetic reprogramming of any nucleated cell utilizing nucleic acid or protein electroporation (for example methods see Gagne et al., 1991; Saito et al., 2001; Yuan, 2008; Huang et al., 2007; Xia and Zhang, 2007; Cemazar and Sersa 2007; Isaka and Imai, 2007; Luxembourg et al., 2007; Van Tendeloos, 2007; Takahashi, 2007; etc.) electroporation, liposomes, nanocapsules, nanovaults, and/or another approach avoiding viral integration or other random alteration of the cell's genome as such means increase safety and efficiency.

In another preferred embodiment, transfection/contacting with long (PRR+) numb isoform encoding sequences (and/or synthetic oligonucleotides targeting numblike and short numb isoforms) is accompanied or replaced by transient or permanent transfection with other sequences including ones selected from those encoding human LIF (e.g. Du and Shi, 1996) oncostatin-M, cardiotrophin-1, IL-I 1, IL-6, IL6R, hyper IL-6, LIFR, gp130, OCT3 (OCT4), Nanog, SOX2, and/or FGF-4.

Simultaneous transfection/contacting with any subset of these distinct transgene sequences can be accomplished by any means known to the art including the use of a single genetic vector, multiple genetic vectors, serial transfection/contacting and selection based on distinct marker proteins and/or antibiotic resistances.

In another preferred embodiment, cells transfected/contacted with long (PRR+) numb isoform(s) are cultured in a cell culture promoting an optimal growth rate, such as described above, and that includes EGF, bFGF, oncostatin, LIF (e.g. Du and Shi, 1996), steel factor, IL-11, cardiotrophin-1, IL-6, hyper-IL-6, CNTF, and/or soluble gpl30.

### Assessment of Potency and Differentiation

Pluripotency and multipotency can be assessed by any means known to the art including 1) transplantation, 2) culture under conditions promoting embryoid body formation, 3) injection of cells into animal blastocyst stage embryos with subsequent development, and 4) RNA expression assays (e.g. RI-PCR and microarray based analyses) for gene expression associated with differentiation, multipotency, pluripotency, etc. (see Guan et al., 2006), 5) colony-formation, as well as by ES-like morphology. One approach disclosed herein for detecting pluripotency in selected cells and/or their progeny involves transfection/contacting with a reporter construct comprising the Nanog promoter operably linked to a fluorescent protein gene. This allows identification and enrichment of Nanog expressing cells using Fluorescence Activated Cell Sorting (FACS), etc.

In a preferred embodiment, endogenous cells (e.g. cells surrounding a burn or injury site) are transfected/contacted in vivo with genetic vectors encoding the long (PRR+) numb isoform(s) alone or in conjuction with other transgenes named herein to transiently promote renewed or increased cell proliferation. This approach can also be utilized clinically in the setting of hypoplastic tissues, disorders where stem/progenitor cells are abnormally depleted, and other disorders where the approach can be shown to be beneficial.

### Achieving Differentiating Cell Populations

In order to achieve b) neural c) muscle d) and other cell populations capable of further environmentally-regulated differentiation in vivo, selected cell(s) and/or their progeny are optionally transfected/contacted with long (PRR+) Numb isoform sequence(s) and/or synthetic oligonucleotide sequences and expanded by growth for sufficient time to achieve the desirable number of cell progeny in vitro (as described above).

Following this optional step, the selected cells and/or their progeny are washed free of the cytokines and agents comprising the expansion/optimal growth media, and are optionally transfected/contacted with the nucleotide sequence(s) encoding the Numblike gene and/or “short” ‘(PRR−) Numb isoform(s) and/or synthetic oligonucleotides targeting the long (PRR+) isoforms, etc. (e.g. Zaehres et al., 2005), then cultured under conditions which promote differentiation of the selected cells into the desired cell type(s).

In most instances, the cells are then cultured in the presence of 5-10% fetal bovine serum and agents(s) promoting differentiation of the selected cells and/or their progeny into a desired cell population. The presence of the fetal bovine and of the agents(s) provides for growth or proliferation at a rate that is less than the optimal (or expansion) growth rate, and favors differentiation of the cells into a desired cell population. The agents and precise culture conditions are selected according to the desired cell population as described below.

### Achieving Neuronal or Neural Cell Populations

When the desired cell population is a neural cell population, the successfully transfected cells are cultured under conditions that promote growth at a rate which is less than the optimal rate and in the presence of agent(s) promoting differentiation of the cells into neural cells. Conditions promoting differentiation into neurons have been described in numerous publications including (Benninger et al., 2003; Chung et al. 2005; Harkany et al., 2004; Ikeda et al., 2004; Ikeda et al., 2005; Wernig et al., 2002; and Wernig et al., 2004). Furthermore, combining retinoic acid exposure with the presence of additional cytokines favors specific neuronal cell type differentiation in vitro (e.g. Soundararajan et al., 2006; Soundararajan et al., 2007; U.S. Pat. No. 6,432,711).

In a preferred embodiment, in vitro differentiation of neurons or neural cells occurs in the presence of 50 ng/mL nerve growth factor (NGF).

In a preferred embodiment, when a neuronal population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding Nurrl, REN, Neurogeninl, Neurogenin2, Neurogenic, Mash 1, Phox2b, Phox2a, dlland, Gata3, Shh, FGF8, Lmxlb, Nkx2.2, Petl, Lbxl, and/or Rnx.

In another preferred embodiment, when dopaminergic neurons are the desired neuronal population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding Mashl, Ngn2, Nurrl, Lmxlb, and/or Ptx-3.

In another preferred embodiment, when serotonergic neurons are the desired neuronal population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding Mashl, Phox2b, Lmxlb, Nkx2.2, Gata2, Gata3 and/or Petl.

In another preferred embodiment, when cholinergic neurons are the desired neuronal population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding MASHlIl, Phox2a and/or RKST4.

In another preferred embodiment, when GABAergic neurons are the desired neuronal population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding MASHl, Phox2a and/or REST4, followed, optionally, by culture in media supplemented with LIF, Neurotrophin 3 (NT3), and/or nerve growth factor (NGF).

In another preferred embodiment, when noradrenergic neurons are the desired neuronal population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding Mashl, dlland, Phox2a, Phox2b, Gata2 and/or Gata3.

In another preferred embodiment, when GABAergic neurons are the desired neuronal population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding P1TX2, Dlx2, Dlx5, antisense Hesl RNA and/or other HESl targeting synthetic oligonucleotides.

In another preferred embodiment, when a neuronal or neural cell population is the desired population, cells transfected/contacted with short (PRR−) numb isoforms (and/or numblike) are cultured in a cell culture medium promoting differentiation, such as described above and that includes one or more of the following agents: retinoic acid, NT3, NGF, glial cell-line derived growth factor (GDNF), and interferon gamma (IFN-gamma)

### Achieving Muscle Cell Populations

When the desired cell population is a muscle population, the successfully transfected/contacted cells are cultured in the presence of an agent promoting differentiation of the cells into muscle cells and growth at a rate less than the optimal rate. Conditions promoting differentiation into muscle cells have also been described previously (Nakamura et al., 2003; Pal and Khanna, 2005; Pipes et al., 2005; Albilez et al., 2006; Pal and Khanna, 2007; Behfar et al., 2007; U.S. Pat. No. 6,432,711). Furthermore, exposure of selected cells and/or their progeny to hexamethylene bis-acrylamide or dimethylsulfoxide in the presence of additional cytokines favors the initiation of muscle type differentiation in vitro.

In a preferred embodiment, when a cardiac muscle cell population is the desired population, cells transfected/contacted with short (PRR−) numb isoforms (and/or numblike) are cultured in a cell culture medium promoting differentiation into cardiomyocytes (He et al., 2003; Guan et al., 2007; etc.), or that includes specific agents at concentrations promoting cardiac cell differentiation (e.g. 0.75%-1% dimethyl sulfoxide (DMSO), 20% normal bovine serum (NBS), 10(-7) mM retinoic acid (RA) and 20% cardiomyocytes conditioned medium (Hua et al., 2006).

In a preferred embodiment, when a muscle cell population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding muscle type specific bHLH-encoding sequences, MyoD, Myogenin, Myf5, Myf6, Mef2, Myocardin, Ifrdl, Gata 4, Gata 5, and Gata 6.

In another preferred embodiment, when a cardiac muscle cell population is the desired population, the cells are transfected/contacted with nucleotide sequences including ones selected from those sequences encoding Myocardin, Gata 4, Gata 5, and Gata 6.

In a preferred embodiment, when a smooth muscle cell population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding the muscle type specific Myocardin nucleotide sequence.

In a preferred embodiment, when a skeletal muscle cell population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding the muscle type specific MyoD and myogenin nucleotide sequences.

In a preferred embodiment, when an oligodendrocyte cell population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding the oligodendrocyte-specific OLIG1, OLIG2, and Zfp488 nucleotide sequences.

Simultaneous transfection with any subset of these distinct transgene sequences listed above can be accomplished by any means known to the art including the use of multiple genetic vectors, serial transfection as well as selection based on distinct marker proteins and/or antibiotic resistance.

When the desired cell population is a hematopoietic cell population, the differentiation medium includes specific agents at concentrations promoting differentiation into hematopoietic progenitor cells (e.g. vascular endothelial growth factor (VEGF), thrombopoietin, etc. (e.g. Ohmizono, 1997; Wang et al., 2005; Srivastava et al., 2007; Gupta et al., 2007) or differentiated hematopoietic cell types (according to methods known to the art for providing differentiated hematopoietic cell types from undifferentiated or pluripotent cells).

When the desired cell population is a germ cell population, the differentiation medium includes specific agents at concentrations promoting differentiation into germ cells (e.g. Nayernia et al. 2006a, 2006b).

In a preferred embodiment, when a germ cell population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding FIGLA (NCBI accession No: AY541030 NM_001004311, SEQ ID NO: 130), FIG alpha (NCBI accession No: U91840, SEQ ID NO: 131), DAZL (NCBI accession No: NM_001190811, SEQ ID NO: 122; NCBI accession No: NM_001351, SEQ ID NO: 123), STRA8 (NCBI accession No: NM_182489, SEQ ID NO: 215), FOXL2 (NCBI accession No: AF522275, SEQ ID NO: 132), OOGENESIN1 (NCBI accession No: NM_178657 XM_622900, SEQ ID NO: 181), OOGENESIN2 (NCBI accession No: NM_198661 XM_355532, SEQ ID NO: 182), OOGENESIN3 (NCBI accession No: NM_201258 XM_131812, SEQ ID NO: 183), OOGENESIN4 (NCBI accession No: NM_001347238 XM_006538858, SEQ ID NO: 184; NCBI accession No: NM_173773, SEQ ID NO: 185), SYCP2 (NCBI accession No: NM_014258 XM_005260247, SEQ ID NO: 216), SYCP3 (NCBI accession No: NM_001177949, SEQ ID NO: 217; NCBI accession No: NM_153694, SEQ ID NO: 218; NCBI accession No: NM_001177948, SEQ ID NO: 219), SPO11 (NCBI accession No: AF169385, SEQ ID NO: 207), REC8 (NCBI accession No: NM_005132, SEQ ID NO: 194; NCBI accession No: NM_001048205, SEQ ID NO: 195), DMC1 (NCBI accession No: NM_007068, SEQ ID NO: 124; NCBI accession No: NM_001363017, SEQ ID NO: 125; NCBI accession No: NM_001278208, SEQ ID NO: 126), MOS (NCBI accession No: NM_005372, SEQ ID NO: 166), STAG3 (NCBI accession No: NM_012447, SEQ ID NO: 212; NCBI accession No: NM_001282716, SEQ ID NO: 213; NCBI accession No: NM_001282717, SEQ ID NO: 214), CCNB1 (NCBI accession No: NM_001354845, SEQ ID NO: 117; NCBI accession No: NM_031966, SEQ ID NO: 118; NCBI accession No: NM_001354844, SEQ ID NO: 119), FOXO1 (NCBI accession No: NM_002015, SEQ ID NO: 133), FOXO3 (NCBI accession No: BC068552, SEQ ID NO: 134), SOHLH1 (NCBI accession No: NM_001101677, SEQ ID NO: 199; NCBI accession No: NM_001012415, SEQ ID NO: 200), SOHLH2 (NCBI accession No: NM_017826 XM_370720, SEQ ID NO: 201; NCBI accession No: NM_001282147, SEQ ID NO: 202), NOBOX (NCBI accession No: NM_001080413, SEQ ID NO: 167; NCBI accession No: XM_017011742, SEQ ID NO: 168), OBOX1 (NCBI accession No: NM_027802, SEQ ID NO: 173), OBOX2 (NCBI accession No: NM_145708, SEQ ID NO: 174), OBOX3 (NCBI accession No: NM_145707, SEQ ID NO: 175), OBOX4 (NCBI accession No: AF461109, SEQ ID NO: 176), OBOX6 (NCBI accession No: NM_145710, SEQ ID NO: 177), LHX8 (NCBI accession No: NM_001001933, SEQ ID NO: 154), LHX9 (NCBI accession No: NM_020204, SEQ ID NO: 155; NCBI accession No: NM_001014434, SEQ ID NO: 156), OOG1 (NCBI accession No: NM_178657 XM_622900, SEQ ID NO: 181), SP1 (NCBI accession No: NM_138473 XM_028606, SEQ ID NO: 208; NCBI accession No: NM_003109, SEQ ID NO: 209), ZFP38 (NCBI accession No: NM_011757, SEQ ID NO: 232; NCBI accession No: NM_001044703, SEQ ID NO: 233; NCBI accession No: NM_001044704, SEQ ID NO: 234), TRF2 (NCBI accession No: U95970, SEQ ID NO: 229), TB2/TRF3 (NCBI accession No: AY457923, SEQ ID NO: 230), TAF4B (NCBI accession No: NM_001293725, SEQ ID NO: 220; NCBI accession No: NM_005640, SEQ ID NO: 221), TAF7L (NCBI accession No: NM_024885, SEQ ID NO: 222), TAF71 (NCBI accession No: NM_001168474, SEQ ID NO: 223), TIA1 (NCBI accession No: NM_022037, SEQ ID NO: 225; NCBI accession No: NM_022173, SEQ ID NO: 226; NCBI accession No: NM_001351508, SEQ ID NO: 227), PHTF1 (NCBI accession No: NM_006608, SEQ ID NO: 189; NCBI accession No: NM_001323041, SEQ ID NO: 190; NCBI accession No: NM_001323042, SEQ ID NO: 191), TNP2 (NCBI accession No: NM_005425, SEQ ID NO: 228), HILS1 (NCBI accession No: NR_024193, SEQ ID NO: 145), DAZL (NCBI accession No: NM_001190811, SEQ ID NO: 122; NCBI accession No: NM_001351, SEQ ID NO: 123), BMP15 (NCBI accession No: NM_005448, SEQ ID NO: 116), PTTG3 (NCBI accession No: NR_002734, SEQ ID NO: 193), AURKC (NCBI accession No: NM_001015878, SEQ ID NO: 114; NCBI accession No: NM_001015879, SEQ ID NO: 115), OTX2 (NCBI accession No: NM_021728, SEQ ID NO: 186; NCBI accession No: NM_172337, SEQ ID NO: 187; NCBI accession No: NM_001270523, SEQ ID NO: 188), SOX15 (NCBI accession No: NM_006942, SEQ ID NO: 203), SOX30 (NCBI accession No: NM_178424, SEQ ID NO: 204; NCBI accession No: NM_007017, SEQ ID NO: 205; NCBI accession No: NM_001308165, SEQ ID NO: 206), FOXR1 (NCBI accession No: NM_181721, SEQ ID NO: 135), ALF (NCBI accession No: NM_001133, SEQ ID NO: 113), OCT4 (NCBI accession No: NM_002701, SEQ ID NO: 178; NCBI accession No: NM_203289, SEQ ID NO: 179; NCBI accession No: NM_001173531, SEQ ID NO: 180), DPPA3/STELLA (NCBI accession No: BC062480, SEQ ID NO: 129), ZFP38 (NCBI accession No: NM_011757, SEQ ID NO: 232; NCBI accession No: NM_001044703, SEQ ID NO: 233; NCBI accession No: NM_001044704, SEQ ID NO: 234), RPS6KA3 (NCBI accession No: NM_004586 XM_939339 XM_944112, SEQ ID NO: 196), HINFP (NCBI accession No: NM_015517, SEQ ID NO: 141; NCBI accession No: NM_198971, SEQ ID NO: 142; NCBI accession No: NM_001243259, SEQ ID NO: 143; NCBI accession No: NM_001351957, SEQ ID NO: 144), NPAT (NCBI accession No: D83243, SEQ ID NO: 169), SP1 (NCBI accession No: NM_138473 XM_028606, SEQ ID NO: 208; NCBI accession No: NM_003109, SEQ ID NO: 209), SP3 (NCBI accession No: NM_003111 XM_092672, SEQ ID NO: 210; NCBI accession No: NM_001017371, SEQ ID NO: 211), HOXA1 (NCBI accession No: NM_005522, SEQ ID NO: 146; NCBI accession No: NM_153620, SEQ ID NO: 147), HOXA7 (NCBI accession No: AJ005814, SEQ ID NO: 148), HEX (NCBI accession No: Z21533, SEQ ID NO: 140), YP30 (NCBI accession No: NM_214493, SEQ ID NO: 231), ZP1 (NCBI accession No: NM_207341, SEQ ID NO: 235), ZP2 (NCBI accession No: NM_003460, SEQ ID NO: 236; NCBI accession No: NM_001290104, SEQ ID NO: 237), ZP3 (NCBI accession No: X56777 S53912, SEQ ID NO: 238), SFE1 (NCBI accession No: NM_001170815, SEQ ID NO: 197), SFE9 (NCBI accession No: AY540956, SEQ ID NO: 198), OPO, PLN (NCBI accession No: NM_002667, SEQ ID NO: 192), RDV, GLD1, MMU-MiR351 (NCBI accession No: NR_029776, SEQ ID NO: 161), MMU-MiR615 (NCBI accession No: NR_030526, SEQ ID NO: 164), MMU-MiR592 (NCBI accession No: NR_030420, SEQ ID NO: 163), MMU-MiR882 (NCBI accession No: NR_030540, SEQ ID NO: 165), MMU-MiR185 (NCBI accession No: NR_029571, SEQ ID NO: 157), MMU-MiR491 (NCBI accession No: NR_030478, SEQ ID NO: 162), MMU-MiR326 (NCBI accession No: NR_029891, SEQ ID NO: 159), MMU-MiR330 (NCBI accession No: NR_029763, SEQ ID NO: 160), MMU-MiR351 (NCBI accession No: NR_029776, SEQ ID NO: 161).

For example, but not limiting, in one preferred embodiment, when a sperm or spermatocyte cell population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding SYCP2 (NCBI accession No: NM_014258 XM_005260247, SEQ ID NO: 216), SYCP3 (NCBI accession No: NM_001177949, SEQ ID NO: 217; NCBI accession No: NM_153694, SEQ ID NO: 218; NCBI accession No: NM_001177948, SEQ ID NO: 219), SPO11 (NCBI accession No: AF169385, SEQ ID NO: 207), REC8 (NCBI accession No: NM_005132, SEQ ID NO: 194; NCBI accession No: NM_001048205, SEQ ID NO: 195), DMC1 (NCBI accession No: NM_007068, SEQ ID NO: 124; NCBI accession No: NM_001363017, SEQ ID NO: 125; NCBI accession No: NM_001278208, SEQ ID NO: 126), MOS (NCBI accession No: NM_005372, SEQ ID NO: 166), STAG3 (NCBI accession No: NM_012447, SEQ ID NO: 212; NCBI accession No: NM_001282716, SEQ ID NO: 213; NCBI accession No: NM_001282717, SEQ ID NO: 214), OCT4 (NCBI accession No: NM_002701, SEQ ID NO: 178; NCBI accession No: NM_203289, SEQ ID NO: 179; NCBI accession No: NM_001173531, SEQ ID NO: 180), ALF (NCBI accession No: NM_001133, SEQ ID NO: 113), RPS6KA3 (NCBI accession No: NM_004586 XM_939339 XM_944112, SEQ ID NO: 196), HINFP (NCBI accession No: NM_015517, SEQ ID NO: 141; NCBI accession No: NM_198971, SEQ ID NO: 142; NCBI accession No: NM_001243259, SEQ ID NO: 143; NCBI accession No: NM_001351957, SEQ ID NO: 144), SP1 (NCBI accession No: NM_138473 XM_028606, SEQ ID NO: 208; NCBI accession No: NM_003109, SEQ ID NO: 209), SP3 (NCBI accession No: NM_003111 XM_092672, SEQ ID NO: 210; NCBI accession No: NM_001017371, SEQ ID NO: 211), TAF71 (NCBI accession No: NM_001168474, SEQ ID NO: 223), TIA1 (NCBI accession No: NM_022037, SEQ ID NO: 225; NCBI accession No: NM_022173, SEQ ID NO: 226; NCBI accession No: NM_001351508, SEQ ID NO: 227), PHTF1 (NCBI accession No: NM_006608, SEQ ID NO: 189; NCBI accession No: NM_001323041, SEQ ID NO: 190; NCBI accession No: NM_001323042, SEQ ID NO: 191), TNP2 (NCBI accession No: NM_005425, SEQ ID NO: 228), HILS1 (NCBI accession No: NR_024193, SEQ ID NO: 145), CLGN (NCBI accession No: NM_001130675, SEQ ID NO: 120; NCBI accession No: NM_004362, SEQ ID NO: 121), TEKT1 (NCBI accession No: NM_053285, SEQ ID NO: 224), FSCN3 (NCBI accession No: NM_020369, SEQ ID NO: 136), DNAHC8 (NCBI accession No: NM_001206927, SEQ ID NO: 127; NCBI accession No: NM_001371, SEQ ID NO: 128), LDHC (NCBI accession No: NM_017448, SEQ ID NO: 149; NCBI accession No: NM_002301, SEQ ID NO: 150), ADAM3 (NCBI accession No: AK302269, SEQ ID NO: 110), OAZ3 (NCBI accession No: NM_016178, SEQ ID NO: 170; NCBI accession No: NM_001134939, SEQ ID NO: 171; NCBI accession No: NM_001301371, SEQ ID NO: 172), AKAP3 (NCBI accession No: NM_001278309, SEQ ID NO: 111; NCBI accession No: NM_006422, SEQ ID NO: 112), MMU-MiR351 (NCBI accession No: NR_029776, SEQ ID NO: 161), MMU-MiR615 (NCBI accession No: NR_030526, SEQ ID NO: 164), MMU-MiR592 (NCBI accession No: NR_030420, SEQ ID NO: 163), MMU-MiR882 (NCBI accession No: NR_030540, SEQ ID NO: 165), and MMU-MiR185 (NCBI accession No: NR_029571, SEQ ID NO: 157).

For example, but not limiting, in one preferred embodiment, when a oocyte cell population is the desired cell population, transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding MOS (NCBI accession No: NM_005372, SEQ ID NO: 166), CCNB1 (NCBI accession No: NM_001354845, SEQ ID NO: 117; NCBI accession No: NM_031966, SEQ ID NO: 118; NCBI accession No: NM_001354844, SEQ ID NO: 119), OCT4 (NCBI accession No: NM_002701, SEQ ID NO: 178; NCBI accession No: NM_203289, SEQ ID NO: 179; NCBI accession No: NM_001173531, SEQ ID NO: 180), FIG alpha (NCBI accession No: U91840, SEQ ID NO: 131), FIGL alpha (NCBI accession No: AY541030 NM_001004311, SEQ ID NO: 130), ALF (NCBI accession No: NM_001133, SEQ ID NO: 113), SOHLH1 (NCBI accession No: NM_001101677, SEQ ID NO: 199; NCBI accession No: NM_001012415, SEQ ID NO: 200), SOHLH2 (NCBI accession No: NM_017826 XM_370720, SEQ ID NO: 201; NCBI accession No: NM_001282147, SEQ ID NO: 202), LHX8 (NCBI accession No: NM_001001933, SEQ ID NO: 154), LHX9 (NCBI accession No: NM_020204, SEQ ID NO: 155; NCBI accession No: NM_001014434, SEQ ID NO: 156), OOG1 (NCBI accession No: NM_178657 XM_622900, SEQ ID NO: 181), FIG alpha (NCBI accession No: U91840, SEQ ID NO: 131), SP1 (NCBI accession No: NM_138473 XM_028606, SEQ ID NO: 208; NCBI accession No: NM_003109, SEQ ID NO: 209), LHX3 (NCBI accession No: NM_178138, SEQ ID NO: 151; NCBI accession No: NM_014564, SEQ ID NO: 152; NCBI accession No: NM_001363746, SEQ ID NO: 153), LHX9 (NCBI accession No: NM_020204, SEQ ID NO: 155; NCBI accession No: NM_001014434, SEQ ID NO: 156), TBP2/TRF3 (NCBI accession No: AY457923, SEQ ID NO: 230), DAZL (NCBI accession No: NM_001190811, SEQ ID NO: 122; NCBI accession No: NM_001351, SEQ ID NO: 123), BMP15 (NCBI accession No: NM_005448, SEQ ID NO: 116), GDF9 (NCBI accession No: NM_005260, SEQ ID NO: 137; NCBI accession No: NM_001288824, SEQ ID NO: 138; NCBI accession No: NM_001288825, SEQ ID NO: 139), PTTG3 (NCBI accession No: NR_002734, SEQ ID NO: 193), AURKC (NCBI accession No: NM_001015878, SEQ ID NO: 114; NCBI accession No: NM_001015879, SEQ ID NO: 115), OTX2 (NCBI accession No: NM_021728, SEQ ID NO: 186; NCBI accession No: NM_172337, SEQ ID NO: 187; NCBI accession No: NM_001270523, SEQ ID NO: 188), SOX15 (NCBI accession No: NM_006942, SEQ ID NO: 203), SOX30 (NCBI accession No: NM_178424, SEQ ID NO: 204; NCBI accession No: NM_007017, SEQ ID NO: 205; NCBI accession No: NM_001308165, SEQ ID NO: 206), FOXR1 (NCBI accession No: NM_181721, SEQ ID NO: 135), NOBOX (NCBI accession No: NM_001080413, SEQ ID NO: 167; NCBI accession No: XM_017011742, SEQ ID NO: 168), OBOX1 (NCBI accession No: NM_027802, SEQ ID NO: 173), OBOX2 (NCBI accession No: NM_145708, SEQ ID NO: 174), OBOX3 (NCBI accession No: NM_145707, SEQ ID NO: 175), OBOX6 (NCBI accession No: NM_145710, SEQ ID NO: 177), OOGENESIN1 (NCBI accession No: NM_178657 XM_622900, SEQ ID NO: 181), OOGENESIN2 (NCBI accession No: NM_198661 XM_355532, SEQ ID NO: 182), OOGENESIN3 (NCBI accession No: NM_201258 XM_131812, SEQ ID NO: 183), OOGENESIN4 (NCBI accession No: NM_001347238 XM_006538858, SEQ ID NO: 184; NCBI accession No: NM_173773, SEQ ID NO: 185), YP30 (NCBI accession No: NM_214493, SEQ ID NO: 231), ZP1 (NCBI accession No: NM_207341, SEQ ID NO: 235), ZP2 (NCBI accession No: NM_003460, SEQ ID NO: 236; NCBI accession No: NM_001290104, SEQ ID NO: 237), ZP3 (NCBI accession No: X56777 S53912, SEQ ID NO: 238), SFE1 (NCBI accession No: NM_001170815, SEQ ID NO: 197), SFE9 (NCBI accession No: AY540956, SEQ ID NO: 198), OPO, PLN (NCBI accession No: NM_002667, SEQ ID NO: 192), RDV, GLD1, DAZL (NCBI accession No: NM_001190811, SEQ ID NO: 122; NCBI accession No: NM_001351, SEQ ID NO: 123), STRA8 (NCBI accession No: NM_182489, SEQ ID NO: 215), MMU-MiR615 (NCBI accession No: NR_030526, SEQ ID NO: 164), MMU-MiR491 (NCBI accession No: NR_030478, SEQ ID NO: 162), MMU-MiR326 (NCBI accession No: NR_029891, SEQ ID NO: 159), MMU-MiR330 (NCBI accession No: NR_029763, SEQ ID NO: 160), MMU-MiR212 (NCBI accession No: NR_029794, SEQ ID NO: 158) and MMU-MiR351 (NCBI accession No: NR_029776, SEQ ID NO: 161).

When the desired cell population is an endoderm and pancreatic islet cell population, the differentiation media includes specific agents at concentrations promoting differentiation into endoderm and pancreatic islet cells (e.g. Xu et al., 2006; Denner et al., 2007; Shim et al., 2007; Jiang et al., 2007).

In a preferred embodiment, differentiation of selected cells and/or their progeny may occur in the differentiation medium in the absence of transfection with numblike, short Numb isoforms or other transgenes/proteins, although the differentiation medium may be unchanged.

In embodiments, a single vector will be utilized which controls the expression of nucleotide sequence(s) encoding the “long” (PRR+) isoform(s) of the mammalian numb gene (and/or synthetic oligonucleotides targeting numblike or the short numb isoforms) under one regulable promoter (e.g. a tetracyc line-regulated promoter), while the Numblike and short Numb isoforms (and/or synthetic oligonucleotides targeting the long (PRR+) isoforms) are expressed under the control of another, distinct, but also regulable promoter. Thus, the long (PRR+) numb isoform(s) can be expressed (and/or short isoforms repressed) when expansion of the selected cells is desired and an inducing agent (e.g. tetracycline) is added to the growth medium; later numblike and the short isoforms can be expressed (and/or long (PRR+) numb isoform(s) repressed) when differentiation is desired.

Alternatively, proteins and peptides corresponding to Numb isoforms, Notch, OCT3/4, SOX2, and other DNA sequences listed herein may be applied in analogous fashion to selected cells and/or their progeny via electroporation (e.g. Koken et al., 1994; Ritchie and Gilroy, 1998), using nano particles, cationic lipids, fusogenic liposomes (e.g. Yoshikawa et al., 2005; 2007), etc. in lieu of, or in combination with genetic transfection. Generally, electroporation allows for high transfection efficiency (and efficient production of the desired cells) without genomic integration of the transgene and is therefore associated with increased safety.

The DNA or RNA encoding protein(s) or polypeptide(s) promoting proliferation, multipotentiality, pluripotentiality or differentiation of the selected cells may be isolated in accordance with standard genetic engineering techniques (for example, by isolating such DNA from a cDNA library of the specific cell line) and placing it into an appropriate expression vector, which then is transfected into the selected cells.

In another preferred embodiment, endoderm and pancreatic islet cells are the desired population, and transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding Foxa2, Sox17, HLXB9 and/or Pdxl.

In another preferred embodiment, hepatocytes are the desired population, and transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding hepatic nuclear factor (HNF)-I, IINF-3, IINF-4, HNF-6 and creb-binding protein.

In another preferred embodiment, hematopoietic cells are the desired population, and transfection/contacting with short numb isoform (and/or numblike) proteins or with sequences encoding short numb isoform proteins (and/or numblike), is accompanied or replaced by transient or permanent transfection/contacting with other proteins and/or nucleic acid sequences, including ones selected from those encoding Runxl/AML1 and NOV(CCN3), and/or cell culture in the presence of colony stimulating factors specific for the desired cell populations. The Runxl/AML1 a isoform is introduced when engraftment is desired and the b isoform when differentiation is desired (Creemers et al., 2006).

In another preferred embodiment, chondrocytes are the desired population, and lransfcction with sequences encoding short numb isoforms (and/or numblike) is accompanied or replaced by transient or permanent transfection of other sequences including ones encoding Sox9, CREB-binding protein, Gataó and/or Runx2.

In another preferred embodiment, bone cells (especially osteoblasts) are the desired population, and transfection with sequences encoding short numb isoforms (and/or numblike) is accompanied or replaced by transient or permanent transfection of other sequences including Runx2.

In a preferred embodiment, the genetic vectors encoding the long Numb isoforms (such as those described herein) are introduced transiently or under the control of a regulable promoter, into endogenous cells in vivo in order to cause those cells proliferate transiently.

In a preferred embodiment, endogenous cells (e.g. ependymal zone cells of the central nervous system) are transfected/contacted in vivo with genetic vectors encoding either the shortest numb isoform or the numblike protein(s) alone or in conduction with other transgenes/proteins named herein, in order to transiently or permanently promote renewed or increased differentiation (especially neuronal differentiation) and migration of progenitor/ependymal cells in the central nervous system). This renewal or increase is measured in terms of the number of cells showing new-onset expression of markers associated with differentiation. This may be accomplished by introduction of the genetic vectors into the organ system using methods suitable for that purpose (see examples).

In a preferred embodiment, endogenous cells (e.g. ependymal zone cells of the central nervous system) are transfected/contacted in vivo with genetic vectors encoding the long numb isoform(s) and/or other transgenes/proteins named herein, in order to transiently promote renewed or increased stem cell proliferation (with subsequent differentiation of progeny cells). This renewal or increase is measured in terms of the number of cells showing new-onset expression of marlers associated with dividing progenitors. This may be accomplished by introduction of the genetic vectors into the organ system using methods suitable for that purpose (see examples).

Likewise this approach is also be suitable for inducing renewed or increased differentiation from other stem cell populations in other tissues (such as the skin, etc). This approach can be utilized, for example, clinically in the setting of central nervous system injury, disorders of other tissues where normal differentiation or migration are inadequate, dysplaslic disorders and other disorders where the approach is beneficial.

In a preferred embodiment, nucleic acid(s) or protein(s) corresponding to a single gene, or portion thereof, (particularly those named herein, discovered according to methods described herein, discovered according to other published methods; and/or known to be capable of initiating the desired manner of differentiation) are the only nucleic acid(s) or protein(s) overexpressed and/or introduced to initiate differentiation in the selected cells.

In a preferred embodiment, nucleic acid(s) or protein(s) corresponding to a single gene, or portion thereof, (particularly those named herein, discovered according to methods described herein, discovered according to other published methods; and/or known to be capable of initiating the desired manner of differentiation) are the only nucleic acid(s) or protein(s) overexpressed and/or introduced to initiate differentiation in the selected cells and the method utilized is electroporation, liposomes, nanocapsules, nanovaults, and/or another approach avoiding retroviral/lentiviral integration or other random alteration of the cell's genome.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; and/or known to be capable of initiating the desirable manner of differentiation) so long as a population of differentiating cells is produced from the selected cells.

In a separate preferred embodiment, other nucleic acid(s) or protein(s) can be utilized in concert with the nucleic acid(s) or protein(s) corresponding to a single gene, or portion thereof, (particularly those named herein, discovered according to methods described herein, discovered according to other published methods; and/or known to be capable of initiating the desirable manner of differentiation) so long as a population of differentiating cells is produced from the selected cells and the method utilized is electroporation, liposomes, nanocapsules, nanovaults, and/or another approach avoiding retroviral/lentiviral integration or other random alteration of the cell's genome.

It is to be understood that any combination of nucleic acid or protein sequences described herein can be modified by excluding those corresponding to Numb and/or Numblike so long as the desired cell population or behavior is achieved.

Similarly, it should be understood that the methods described herein (or elsewhere) for initiating differentiation are applicable to any induced or non-induced multipotent, pluripotent, or self-renewing stem cells, or other selected cells, not only those obtained in the manner described herein.

### Sources of Selected Cells

The population of selected cells may derive from various stem cells, progenitor cells and somatic cells. However somatic cells lacking nuclei (e.g. mature, human red blood cells) are specifically excluded. Selected stem cells may be derived from existing cell lines or isolated from stored, banked, or cryopreserved sources. Typical sources of stem cells include bone marrow, peripheral blood, placental blood, amniotic fluid (e.g. De Coppi et al., 2007), umbilical cord blood (e.g. Zhao, et al., 2006; Tian et al., 2007), adipose tissue (e.g. Gimble et al., 2007; Ma et al., 2007), non-human embryos, and others. Circulating leukocytes and other non-stem cells may likewise be selected and subjected to the same culture conditions as described above effective that they acquire multipotency, pluripotency and/or self-renewal as a result. Examples of other accessible somatic cells useful in this invention include lymphocytes and epithelial (e.g. buccal cheek) cells. Isolation and collection of cells selected for use within the present invention may be performed by any method known to the art.

In embodiments involving animals, stem cells isolated from prostate, testis, embryonic brain, and intestine are also disclosed as being preferred sources of selected cells.

In a preferred embodiment, the selected cells and/or their progeny are cultured in a three-dimensional format.

A further aim of the present invention is to provide cells for use in the production of patient-compatible and patient-specific tissues and organs for transplantation to patients deemed to be requiring such organs or tissues. It is disclosed herein that the pluripotent, multipotent, and/or differentiating cells provided by the methods described herein (or similar methods) be utilized in conjunction with techniques aimed at the production of such organs and/or tissues (e.g. Boland et al., 2006. Xu et al., 2006; Campbell and Weiss, 2007). Such utilization is specifically covered by the present invention.

For instance, pluripotent, multipotent, and/or differentiating cells produced or treated according to the methods desribed herein (or other published methods) may be grown in association with three-dimcsnisonal or two-dimensional scaffoldings engineered to replicate normal tissue structure and/or organ structures (e.g. Yarlagada et al., 2005; Kim et al, 1998; WO/2003/070084; EP1482871; WO03070084; U.S. Pat. Nos. 2,395,698; 7,297,540; 6,995,013; 6,800,753; Isenberg et al., 2006).

Similarly, scaffoldings to be occupied by the pluripotent, multipotent, and/or differentiating cells may be derived from cadaveric organ(s) or tissue(s) after the cadaveric organs or tissues (e.g. bone, heart, kidney, liver, lung, etc.) may be treated in such away that the host immune cells resident in that tissue, and other undesirable or ancillary host cells, are eliminated (e.g. by ionizing radiation, sterilization (e.g. Mroz et al., 2006), and/or various methods of decellularization (U.S. Pat. Nos. 6,734,018; 6,962,814; 6,479,064; 6,376,244; 5,032,508; 4,902,508; 4,956,178; 5,281,422, 5,554,389; 6,099,567; and 6,206,931; 4361552 and 6576618; 6753181; U.S. application Ser. No. 11/162,715; WO/2001/048153; WO/2002/024244; WO003002165; WO/2001/049210; WO/2007/025233; European Patents EP1482871; EP1246903; EP1244396; EP0987998; EP1244396; EP1 333870; Rieder et al., 2004; Ott et al., 2008; Taylor et al., 1998)).

Likewise, it is anticipated that the pluripotent, multipotent, and/or differentiating cells of the present invention may be used in applications utilizing inkjet-style printing for tissue engineering (e.g. Boland et al., 2006. Xu et al., 2006; Campbell et al., 2007).

Therefore such use of the cells produced or treated according to the methods described herein is covered.

In another preferred embodiment, the selected cells and/or their progeny are cultured in hanging drops.

In accordance with another aspect of the present invention, selected cells may be modified genetically beforehand.

In accordance with another aspect of the present invention, selected cells may be modified with DNA or RNA encoding protein(s) or polypeptide(s) promoting differentiation of the cell into a desired cell population.

### Screening Cell Populations

In one embodiment, the methods of this invention comprise screening cells from cell lines, donor sources, umbilical cord blood, and autologous or donor bone marrow, blood, spermatogonia, primordial germ cells, buccal cheek cells, or any other cell source effective in the current invention. Selected cells can be screened to confirm successful transfection with beneficial sequence(s) or therapeutic vector(s) as well as successful initiation of differentiation by any method known to the art (Guan et al., 2006; U.S. Pat. No. 6,432,711). In some embodiments, the cells are screened using standard PCR and nucleic acid hybridization-based methods or using rapid typing methods. In preferred embodiments, the cells are screened according to expression of reporter genes. In some embodiments, cells are screened by expression of a marker gene encoded by the transgene expressing vector(s) such as an antibiotic resistance gene or a fluorescent protein (e.g. GFP) gene.

### Screening for Therapeutic Vectors and Beneficial Sequences

Cells can be screened for the presence of beneficial sequence(s) and therapeutic vector(s) using any method(s) known to the art for detection of specific sequences. Each cell sample can be screened for a variety of sequences simultaneously. Alternatively, multiple samples can be screened simultaneously.

Cell differentiation may be monitored by several means: including (i) morphological assessment, (ii) utilizing reverse transcriptase polymerase chain reaction (RT-PCR), Northern blot, or microarray techniques to monitor changes in gene expression, (iii) assaying cellular expression of specific markers such as beta tubulin III (for neurons) etc. (Ozawa, et al., 1985). In some embodiments, the cells are screened for successful initiation of differentiation using FACS sorting based on cell type specific markers or transgenic marker expression (e.g. antibiotic resistance or fluorescent protein expression) under the control of cell type specific promoters such as the myosin promoter in muscle cells; the human cardiac a-actin promoter in cardiomyocytes; the insulin promoter in insulin producing cells; the neuronal-specific enolase (NSE) promoter for neuronal differentiation, or neurotransmitter related promoters such as the tyrosine hydroxylase promoter in dopaminergic neurons; etc.). In some embodiments, the cells are screened using standard PCR and nucleic acid hybridization-based methods. In a particularly preferred embodiment, the cells are screened using rapid typing methods.

### Screening for Human Leukocyte Antigen (HLA) Type

In certain embodiments, the selected cells are selected with respect to compatible HLA typing. The HLA genotype can be determined by any means known to those of skill in the art.

The cells used for screening may consist of cells taken directly from a donor, or from cell lines established from donor cells, or other practicable cell sources. The cells can be screened for beneficial sequence(s), and/or therapeutic vector(s) and HLA type at once, or separately. Those cells successfully transfected with a beneficial sequence and showing an appropriate HLA genotype can be prepared for transplantation to a patient.

In certain embodiments, the transfected/contacted cells are transplanted without HLA typing. In other embodiments, the cells are HLA typed for compatibility.

### Screening for Agents Promoting a Cellular Phenotype

The present invention also provides for methods of screening proteins and agents for their ability to induce phenotypic changes or differentiation of the selected cells and/or their progeny into desired cell populations. Briefly, vectors encoding complementary DNAs (cDNAs) from appropriate cDNA libraries are transfected into the selected cells/and or their progeny. Once a specific cDNA that induces differentiation or other phenotypic change is identified, such cDNA then may be isolated and cloned into an appropriate expression vector for protein production in appropriate cells (e.g. COS cells) in vitro. Later the protein containing supernatant can be applied to the selected cell cultures to determine if any secreted proteins from such cells induce differentiation Alternatively, candidate agents can be applied to the selected cell cultures to determine if any secreted proteins from such cells induce differentiation (see U.S. Pat. No. 6,432,711).

The present invention also provides for methods of screening nucleic acids for their ability to induce multipotentiality, pluripotentiality, and/or self-renewal, or to initiate differentiation of selected cells and/or their progeny. In these methods, vectors encoding selected cDNAs (or cDNAs from appropriate cDNA libraries, or other sequences) are introduced into the selected cells/and or their progeny using electroporation, nanocapsules, nanovaults, liposomes, retroviruses, lentiviruses, and/or any other practicable means of transfection. Once a specific cDNA that induces a phenotypic change, multipotentiality, pluripotentiality, and/or self-renewal, is identified, such cDNA then may be isolated and cloned into an appropriate expression vector. Assays for determining such changes include those described elsewhere herein.

Likewise the protein corresponding to the identified cDNA may be produced in appropriate cells (e.g. COS cells) in vitro to determine whether the protein containing supernatant can be applied to the selected cell cultures and induce the desired changes.

Finally, proteins may be introduced into the selected cells/and or their progeny using electroporation, nanocapsules, nanovaults, liposomes, retroviruses, lentiviruses, and/or any other practicable means of transfection, and the resulting cells assessed as described herein for multipotentiality, pluripotentiality, self-renewal or the initiation of differentiation.

Tranplantation of Cells into Patients

After screening, selected cells and/or their progeny may be cryopreserved, maintained as cell lines in culture, or may be administered to the patient. Selected cells can be cryopreserved or maintained in culture by any means known to the art and preserved for future transplantation procedures.

Preferably, the cells to be screened are obtained from accessible sources allowing easy collection.

With regard to producing HIV resistant cells: targeted somatic cells and stem cells of this invention can be of any type capable of differentiating into cells that can be infected by HIV, that can sustain the transcription and/or replication of HIV, that can alter the HIV immune response, or that can retard progression to AIDS. Such stem cells include, but are not limited to, pluripotent cells derived from spermatogonia, primordial germ cells, hematopoietic stem cells, peripheral blood cells, placental blood cells, amniotic fluid cells, umbilical cord blood cells, buccal cheek cells, adipose tissue cells (including stem cells derived from those tissues), reprogrammed cells, induced multipotent cells, induced pluripotent cells, etc., non-human embryos, and/or any other cell type that can form blood and immune cells, HIV target cells, and other cells.

Therapeutic vector(s) express “beneficial sequence(s)” intended to render transfected/contacted or infected cells less capable of sustaining HIV replication and transcription. The genetic vector expressing “beneficial sequence(s)” as well as any virus derived from such genetic vector, are herein termed “therapeutic vector”.

After screening, cells transfected/contacted with the desired therapeutic vector(s) and expressing beneficial sequence (with or without compatible HLA genotype) may be expanded ex vivo (in vitro) using standard methods to culture dividing cells and maintained as stable cell lines (U.S. Pat. Nos. 6,432,711 and 5,453,357 herein incorporated by reference). Alternatively, these cells can be administered to the patient and expanded in vivo.

Selected cells can be cryopreserved by any means known to the art and preserved for future transplantation procedures.

Transplantation of Desirable Cell Populations into Patients

In certain embodiments, cell populations are enriched for stem cells prior to transplantation. Various methods to select for stem cells are well known in the art. For example, cell samples can be enriched by fluorescently labeled monoclonal antibodies recognizing cell-surface markers of undifferentiated hematopoietic stem cells (e.g., CD34, CD59, Thyl, CD38 low, C-kit low, lin− minus) for sorting via fluorescence-activated cell sorting (FACS).

In other embodiments, a sample of the selected cells is transplanted, without enrichment.

In some embodiments, the endogenous stem cells of the bone marrow are eliminated or reduced prior to transplantation of the therapeutic stem cells. Therapeutic stem cells are defined as those stem cells containing beneficial sequence(s) or therapeutic vector(s).

In some embodiments, the transplantation process may involve the following phases: (1) conditioning, (2) stem cell infusion, (3) neutropenic phase, (4) engraftment phase, and (5) post-engraftment period.

In some embodiments, the endogenous stem cells that normally produce the desired cells (e.g. bone marrow stem cells) are eliminated or reduced prior to transplantation.

Chemotherapy, radiation, etc. and/or methods analogous to those described in U.S. Pat. No. 6,217,867 may be used to condition the bone marrow for appropriate engraftment of the transplant. Finally, therapeutic stem cells may be transplanted into the patient using any method known to the art.

### Design of Numb/Numblike and Other Transgene Encoding Vectors

In one embodiment transfection with nucleic acid sequence(s) encoding numblike/numb isoform(s) is accomplished via viral transfection. The term “‘Numb/Numblike encoding vector(s)” refers to the vectors incorporating the nucleic acid sequence(s) encoding numblike/numb isoform(s) and/or synthetic oligonucleotides targeting numblike or numb isoforms, as well as any additional transgene sequences, synthetic oligonucleoties, etc, and any associated viral supernatant incorporated in those vector sequences.

### The Numb/Numblike Encoding Vector(s) May Comprise an Expression Vector.

Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to adenoviral vectors, adeno-associated viral vectors, and retroviral vectors. Examples of retroviral vectors which may be employed include, but are not limited to, those derived from Moloney Murine Leukemia Virus, Moloney Murine Sarcoma Virus, and Rous Sarcoma Virus, FIV, HIV, SIV and hybrid vectors.

It is disclosed that the Numb/Numblike encoding vector(s) may be used to transfect cells in vitro and/or in vivo. Transfection can be carried out by any means known to the art, especially through virus produced from viral packaging cells. Such virus may be encapsidated so as to be capable of infecting a variety of cell types. Nevertheless, any encapsidation technique allowing infection of selected cell types and/or their progeny is practicable within the context of the present invention.

### Design of Human Immunodeficiency Virus (HIV) Gene Therapy Vector(s)

The “therapeutic vector(s)” may incorporate an expression vector. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to adenoviral vectors, adeno-associated viral vectors, and retroviral vectors. Examples of retroviral vectors which may be employed include, but are not limited to, those derived from Moloney Murine Leukemia Virus, Moloney Murine Sarcoma Virus, and Rous Sarcoma Virus, feline immunodeficiency virus (FIV), HIV, simian immunodeficiency virus (SIV) and hybrid vectors.

It is disclosed herein that the therapeutic vector(s) may be used to transfect target cells in vitro and/or in vivo. Transfection can be carried out by any means known to the art, especially through virus produced from viral packaging cells. Such virus may be encapsidated so as to be capable of infecting CD34+ cells and/or CD4+ cells. However, in some instances, other cell types are transfected/contacted by means not involving the CD4 or CD34 proteins. Nevertheless, any encapsidation technique allowing infection of such cell types may therefore be included in the disclosure of the present invention.

Pseudotyping with different envelope proteins expands the range of host cells transduceable by viral vectors and therapeutic vectors, and allows the virus to be concentrated to high titers, especially when pseudotyped with the vesicular stomatitis virus envelope glycoprotein (VSV-G) (Li et al., 1998; Reiser et al., 2000).

### Vector Construction

Viral vectors utilized in this invention may be of various types including hybrid vectors. Vectors may, for instance, be third-generation lentiviral vectors which include only a very small fraction of the native genome (Zufferey et al., 1998). Production of transgene encoding vector(s) may also involve self-inactivating transfer vectors (Zufferey et al., 1998; Miyoshi et al., 1998) eliminating the production of full-length vector RNA after infection of target cells.

Viral vectors may be utilized which are replication-incompetent due to failure to express certain viral proteins necessary for replication. However the possibility exists that helper virus may enable therapeutic virus replication. This likelihood can be reduced by the use of self-inactivating vectors.

In a preferred embodiment, transgene sequences are driven by a ubiquitin promoter, U6 promoter, EFl alpha promoter, CMV promoter, regulable promoters and/or desired cell type specific promoters.

### Viral Tropism

In a preferred embodiment, virus derived from the Numb isoform/Numblike encoding vector(s), therapeutic vector(s) and/or other transgeneic vector(s) of this invention is pseudotyped with vesicular stomatitis virus envelope glycoprotein to enable concentration of the virus to high titers and to facilitate infection of CD34+ cells.

### Sequence Selection

The use of any sequence with 70% or greater identity (or complementarity) to any sequence referred to as a NUMB or Numblike sequence (searchable using the Entrez-Pubmed database) is covered by the invention if utilized in the manner described in the present invention.

The current invention also relates in part to a genetic vector that includes sequences capable of markedly reducing the susceptibility of mammalian cells to infection by HIV 1 and IIlV-2 viruses (both together referred to herein as HlV).

The current invention discloses the novel combination of synthetic oligonucleotides to reduce the expression of genes critical to the HIV/AIDS disease process.

The desirability of combining synthetic oligonucleotides to effect co-receptor “knock down” with expression of TAR and RRIi decoy secμjences arises from the proposition, expressed herein, that combining multiple gene therapy approaches simultaneously targeting 1) HIV infection, 2) HlV transcription, and 3) HIV replication in individual cells is likely to produce superior therapeutic benefits than any of these approaches in isolation.

Therapeutic vector(s) express “‘beneficial sequence(s)” intended to render transfected/contacted or infected cells less capable of sustaining HIV replication and transcription. The genetic vector expressing “beneficial sequence(s)” as well as any virus derived from such genetic vector, are herein termed “therapeutic vector”.

The present invention is directed in part to the genetic modification of cells susceptible to infection by HIV or capable of propagating HlV. Such cells are herein termed “target cells”.

The present invention provides a composition and method for using therapeutic viral vectors to reduce the susceptibility of mature or immature target cells, leukocytes, blood cells, any stem/progenitor cells, and/or their progeny to infection by HIV.

It follows that the present invention also provides a composition and method for using therapeutic viral vectors to reduce the susceptibility of reprogrammed cells, induced multipotent cells, induced pluripotent cells, and/or their progeny to infection by HIV.

It is a further objective of this invention to reduce the ability of mature or immature target cells, stem/progenitor cells, (including reprogrammed cells, induced multipotent cells, induced pluripotent cells) and/or their progeny to sustain immunodeficiency virus replication and transcription.

It is another objective of this invention to achieve efficient, long-term expression of the therapeutic sequences in mature or immature target cells, other quiescent cells, stem/progenitor cells, and/or their progeny.

In one aspect, this invention provides a method for preventing or treating HIV infection. The method involves transplanting stem cells transfected/contacted with therapeutic vector(s) or sequence(s), into patients with HIV infection.

Beneficial sequence(s) may be ones that reduce the ability of HIV to infect a cell, transcribe viral DNA, or replicate within an infected cell, or which enhances the ability of a cell to neutralize HIV infection.

In certain embodiments, the beneficial sequence(s) represent synthetic oligonucleotide(s) which interfere with HIV entry, including siRNA, shRNA, antisense RNA or miRNA directed against any of the HIV co-receptors (including, but not limited to, CXCR4, CCR5, CCR2b, CCR3, and CCR1).

In a preferred embodiment, the therapeutic vector(s) includes synthetic oligonucleotides targeting one or more HIV co-receptors including CXCR4, CCR5, CCR1, CCR2, CCR3, CXCR6 and/or BOB.

In another preferred embodiment the therapeutic vector(s) includes synthetic oligonucleotides targeting the major HIV co-receptors CXCR4 and CCR5

In a further preferred embodiment the therapeutic vector(s) includes synthetic oligonucleotides targeting one or more HIV enzymes such as HIV reverse transcriptase, integrase and protease.

Appropriate sequences for the synthetic oligonucleotides are those 1) predictable by computer algorithms to be effective in reducing targeted sequences, and 2) capable of successfully reduce the amount of targeted enzyme by >70% in standard quantitative RNA assays and in assays of enzymatic activity or to a lesser but therapeutic degree.

The phrase “targeted sequence” indicates that a particular sequence has a nucleotide base sequence that has at least 70% identity to a viral genomic nucleotide sequence or its complement (e.g., is the same as or complementary to such viral genomic sequence), or is a corresponding RNA sequence. In particular embodiments of the present invention, the term indicates that the sequence is at least 70% identical to a viral genomic sequence of the particular virus against which the oligonucleotide is directed, or to its complementary sequence.

Any of the various types of synthetic oligonucleotides may be expressed via therapeutic vector transfection, and the current invention is directed to all possible combinations of such oligonucleotides.

In a preferred embodiment, the synthetic oligonucleotide sequences are driven by target cell, specific promoter(s).

In another preferred embodiment, the synthetic oligonucleotide sequences are driven by U6 promoter(s).

Synthetic oligonucleotides, by the same token, may be included in the same therapeutic vector(s) with decoy RNA.

### Decoy RNA

Decoy RNA are sequences of RNA that are effective at binding to certain proteins and inhibiting their function.

In a preferred embodiment, the therapeutic vector(s) comprise(s) multiple decoy RNA sequences.

In a further embodiment the decoy RNA sequences are flanked by sequences that provide for stability of the decoy sequence.

In another preferred embodiment the decoy RNA sequences are RRE and/or TAR decoy sequences.

In a preferred embodiment, the RRE and TAR decoy sequences are HIV-2 derived TAR and RRE sequences.

In another preferred embodiment the decoy sequences also include Psi element decoy sequences.

In a preferred embodiment, the decoy sequences are each driven by a U6 promoter.

In another preferred embodiment, the decoy sequences are driven by target-cell specific promoters.

In a preferred embodiment, the therapeutic vector targets multiple stages of the HIV life cycle by encoding synthetic nucleotide sequence(s) in combination with HIV-2 TAR and/or RRE decoy sequences.

In another preferred embodiment, the vector includes miRNA oligonucleotide sequences.

In another preferred embodiment, the vector includes shRNA oligonucleotide sequences.

In another preferred embodiment, the vector includes si RNA oligonucleotide sequences.

In another preferred embodiment, the vector includes RNAi oligonucleotide sequences.

In another preferred embodiment, the vector includes ribozyme sequences.

In another preferred embodiment, the vector includes a combination of synthetic oligonucleotide classes.

In a further embodiment, the synthetic nucleotide sequences target HIV co-receptors such as CCR5, CXCR4, etc.

In a further embodiment, the synthetic nucleotide sequences target HIV enzymes such as integrase, protease, reverse transcriptase, TAT, etc.

In a further embodiment, the ribozyme sequences target HIV co-receptors such as CCR5, CXCR4, etc, or HIV enzymes such as integrase, protease, reverse transcriptase, TAT, etc.

In a preferred embodiment, virus is generated using the therapeutic vector(s) and the virus is pseudotyped.

In a preferred embodiment, virus is generated using the therapeutic vector(s) and the virus is not pseudotyped and the virus shows native HIV tropism.

In a preferred embodiment, the therapeutic vector(s) is a viral vector.

In a preferred embodiment, the therapeutic vector(s) is a lentiviral vector.

In a preferred embodiment, the therapeutic vector(s) is a third generation lentiviral vector.

In a preferred embodiment, the therapeutic vector(s) includes a combination of synthetic oligonucleotide classes.

In a preferred embodiment, synthetic nucleotide sequence expression is driven by the EF-I alpha promoter or other target-cell appropriate promoters.

In a preferred embodiment, synthetic nucleotide sequence expression is driven by the 1)6 promoter or other target-cell appropriate promoters.

In a preferred embodiment, synthetic nucleotide sequence expression is driven by a combination of EF-I alpha and U6, and/or other target-cell appropriate promoters.

In a preferred embodiment, HF-I alpha drives miRNA expression while the U6 promoter drives RNA decoy expression.

In a preferred embodiment, HF-I alpha drives siRNA sequence expression while the U6 promoter drives RNA decoy expression.

In a preferred embodiment, EF-I alpha drives shRNA sequence expression while the U 6 promoter drives RNA decoy expression.

In a preferred embodiment, the therapeutic vector(s) includes multiple miRNA sequences directed against CXCR4, multiple miRNA sequences directed against CCR5, an 111V-2 RRE decoy sequence and an HIV-2 TAR decoy sequence, and the vector is a viral vector.

In a preferred embodiment, treatment involving the therapeutic vector(s) is combined with other modes of antiretroviral therapy including pharmacological therapies.

Antiretroviral therapies appropriate for combination with the therapeutic vector(s) are those that have additive or synergistic effects in combination with the therapeutic vector.

Cells targeted for gene therapy in HIV may include, but are not necessarily be limited to mature peripheral blood T lymphocytes, monocytes, tissue macrophages, T cell progenitors, macrophage-monocyte progenitor cells, and/or multipotent hematopoietic stem cells, such as those found in umbilical cord blood, peripheral blood, and occupying bone marrow spaces.

The present invention also relates to transfection of CD4+ T cells, macrophages, T cell progenitors, macrophage-monocyte progenitors, CD 34+ stem/progenitor cells and/or any other quiescent cell, dividing cell, stem cell or progenitor cell capable of differentiation in vitro or in vivo into HIV target cells, CD4+ T cells, macrophages, T cell progenitors, macrophage-monocyte progenitors, and/or CD 34+ stem/progenitor cells. Transfected cells, therefore, can be endogenous cells in situ, or exogenous cells derived from other body regions or even other individual donors. Cells selected for this purpose are herein termed “selected cells”.

By the same token, self-renewing, multipotent and/or pluripotent stem cells (including reprogrammed and induced pluripotent cells) represent another logical target for HIV gene therapy, and their use is specifically covered by the present invention.

In one embodiment of this process, selected cells (e.g. hematopoietic stem cells, skin stem cells, umbilical cord cells, primordial germ cells (PGCs), spermatogonia, any accessible somatic cell, etc.) are 1) propagated in culture using one or more cytokines such as steel factor, leukemia inhibitory factor (LIF), cardiotropic 1, IL-11, IL-6, IL-6 R, GP-130, CNTF, IGF-I, bFGF, and/or oncostatin-M and 2) transfected/contacted with the therapeutic vector(s) or beneficial sequence(s) prior to differentiation using any methods known to the art, such as those described in U.S. Pat. No. 5,677,139 herein incorporated by reference, or by methods analogous to U.S. Pat. No. 5,677,139 with respect to other target cells.

In separate embodiments, it may be desirable to perform the various steps prior to transfection.

In separate embodiments, for the purpose of generating pluripotent stem cell populations, it may be desirable to perform only the incubation steps above.

Appropriate concentrations of LlF and steel factor for stem/progenitor cell propagation/proliferation as well as other cell culture conditions have been described previously (e.g. U.S. Pat. Nos. 6,432,711 and 5,453,357 herein incorporated by reference). Other appropriate protocols and reference cytokine concentrations have been taught by Koshimizu et al., 1996; Keller et al., 1996; Piquet-Pellorce, 1994; Rose et al., 1994; Park and Han, 2000; Guan et al., 2006; Dykstra et al., 2006).

The population of target cells may include somatic cells, stem cells and progenitor cells. Stem cells may be derived from existing cell lines or isolated from stored, banked, or cryopreserved sources. Typical sources of stem cells include marrow, peripheral blood, placental blood, amniotic fluid, umbilical cord blood, adipose tissue, non-human embryos, etc.

Somatic cells, especially circulating leukocytes and other non-progenitor/stem cells may likewise be subjected to the same culture conditions as described above for stem/progenitor cells effective that they acquire stem/progenitor cell properties as a result.

The invention also discloses the production (e.g. US Patent Application 20030099621) of target cells from stem/progenitor cells that may be made relatively resistant to HIV infection and/or HIV replication.

It is understood, however, that any method of differentiating previously propagated stem/progenitor/leukocyte cells into the desired target cells may be employed within the scope of the invention so long as functional target cells relatively resistant to HIV infection and/or HIV replication/and/or HIV transcription are produced.

In a preferred embodiment, the therapeutic viral vector is packaged with one or more envelope proteins from native HIV viruses conferring upon the therapeutic virus the capacity to infect any cell that native HIV strains are capable of infecting.

Cells selected for use in this invention will be in some instances accessible (e.g. umbilical cord stem cells, bone marrow stem cells, spermatogonia and primordial germ cells of the testis, stem cells isolated from amniotic fluid, stem cells isolated from the skin, etc.). Such cells can be isolated from the tissues in which they reside by any means known to the art.

Other selected cells may comprise reprogrammed cells, induced multipotent cells, induced pluripotent cells, etc.

In accordance with an aspect of the present invention, there is provided a method of producing a desired cell line, cell type, or cell class from the selected cells. Generally, the method comprises culturing the selected cells and/or their progeny under conditions which promote growth of the selected cells at an optimal growth rate. The resulting cell population is then cultured under conditions which promote cell growth at a rate which is typically less than the optimal rate, and in the presence of an agent promoting differentiation of the cells into the desired cell line, cell type, or cell class (e.g. CD4+ T cells).

The present invention also discloses the propagation of the selected cells and/or their progeny in culture, before or after transfection with the therapeutic vector, by any means known to the art (e.g. US Patent Application 20060099177). Such methods also include incubation with LIF, steel factor, 11-6, IL-7, oncostatin-M and/or cardiotropic 1 and other growth enhancing cytokines, etc.

The present invention further discloses the directed differentiation of cells transfected/contacted with the therapeutic vector(s) into desired cell types by further incubation in media containing the appropriate cytokines and growth factors such as colony stimulating factors such as M-CSF (CSF-I), GM-CSF, IL-7, any cytokine promoting CD4+ T cell differentiation, etc.

### Transfection

Genetic modification of selected cells and target cells, whether they be exogenous cells or endogenous cells can be performed according to any published or unpublished method known to the art (e.g. U.S. Pat. Nos. 6,432,711, 5,593,875, U.S. 5,783,566, U.S. Pat. Nos. 5,928,944, 5,910,488, 5,824,547, etc.) or by other generally accepted means. Suitable methods for transforming host cells can be found in Sambrook et al.

(Molecular Cloning: A Laboratory Manual, 2nd Edition, Cold Spring Harbor Laboratory press (1989)), and other laboratory textbooks.

Successfully transfected/contacted cells can be identified by selection protocols involving markers such as antibiotic resistance genes in addition to RNA expression assays and morphological analyses. Clones from successfully transfected/contacted cells, expressing the appropriate exogenous DNA at appropriate levels, can be preserved as cell lines by cryopreservation (utilizing any appropriate method of cryopreservation known to the art).

Selectable markers (e.g., antibiotics resistance genes) may include those which confer resistance to drugs, such as G418, hygromycin, ampicillin and blasticidin, etc. Cells containing the gene of interest can be identified by drug selection where cells that have incorporated the selectable marker gene survive, and others die.

A theoretical basis for the embodiments of the invention is described herein, however, this discussion is not in any way to be considered as binding or limiting on the present invention. Those of skill in the art will understand that the various embodiments of the invention may be practiced regardless of the model used to describe the theoretical underpinnings of the invention.

The invention will now be described and illustrated with respect to the following examples; however, the scope of the present invention is not intended to be limited thereby.

### Example 1: Construction of the Transgenic Vectors Suitable for Use in the Present Invention

Suitable EGFP-Numb and HGFP-Numblike, and EGFP-X lentiviral vectors (where X is any transgene described in the present invention) can be produced by cloning into an appropriate viral vector (e.g. the two-gene I Il V-EGFP-HSA vector (Reiser et al., 2000)). Adapter primers can be selected for PCR amplification of Numblike and Numb isoform cDNAs and cloning into a genetic vector. In preparation for cloning, the gene vector is digested with enzymes. Subsequently, the cDNA for each transgene is inserted into the nef coding region previously occupied by the I ISA cDNA. EGFP (enhanced green fluorescent protein) and a cell population-appropriate promoter (e.g. CMV ie or EFl alpha) having been previously inserted into the viral coding region. Genetic constructs may include a vector backbone, and a transactivator which regulates a promoter operably linked to heterologous nucleic acid sequences.

Examples of retroviral vectors which may be employed include, but are not limited to, those derived from Moloney Murine Leukemia Virus, Moloney Murine Sarcoma Virus, and Rous Sarcoma Virus, FIV, and HIV. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to, lentiviral vectors, adenoviral vectors, adeno-associated viral vectors, and retroviral vectors.

The replication incompetent pcDNA 6.2/EmGFP-Bsd/V5-DEST vector is an example of an appropriate expression vector (Invitrogen) and allows expression of synthetic oligonucleotides (e.g. miRNAs) transferred from the pcDNA 6.2 GW/miR vector that have the capacity to cleave targeted sequences. These vectors include flanking and loop sequences from endogenous miRNA to direct the excision of the engineered miRNA from a longer Pol II transcript (pre-miRNA).

Combining multiple miRNA sequences directed against specific endogenous RNA species increases the likelihood of success in reducing target sequence expression. miRNA sequences may be operably linked to regulable or tissue specific promoters.

By utilizing lentiviral vectors for gene expression, the resulting Numb/Numblike encoding vector(s) and/or other transgenic vector(s) of this invention, becomes capable of stably transducing both dividing and non-dividing cell types.

In a preferred embodiment, the resulting Numb/Numblike encoding vector(s), and/or other transgenic vector(s) of this invention contain multiple synthetic oligonucleotide sequences driven by one or more promoters so as to reduce expression of specific numb isoforms and/or numblike.

### Example 2

Another example of a suitable vector is a retroviral vector. Retroviruses are RNA viruses that contain an RNA genome. The gag, pol, and env genes are flanked by long terminal repeat (LTR) sequences. The 5′ and 3′ LTR sequences promote transcription and polyadenylation of mRNA's.

The retroviral vector may provide a regulable transactivating element, an internal ribosome reentry site (IRHS), a selection marker, and a target heterologous gene operated by a regulable promoter.

Alternatively, multiple sequences may be expressed under the control of multiple promoters. Finally, the retroviral vector may contain cis-acting sequences necessary for reverse transcription and integration. Upon infection, the RNA is reverse transcribed to DNA that integrates efficiently into the host genome. The recombinant retrovirus of this invention is genetically modified in such a way that some of the retroviral, infectious genes of the native virus have been removed and in certain instances replaced instead with a target nucleic acid sequence for genetic modification of the cell. The sequences may be exogenous DNA or RNA, in its natural or altered form.

### Example 3: Example Methods for Generation of Numb/Numblike Encoding Vector(s), and/or Other Transgenic Vector(s) of this Invention

The methods for generation of the resulting Numb/Numblike encoding vector(s), and/or other transgenic vector(s) of this invention include those taught in Invitrogen's Viral Power Lentiviral Expression Systems Manual, 2007. Briefly, the EmGFP-bsd cassette is cloned as a PmIl-BIpI fragment into the pLenti6/R4R2/V5-DEST vector, while the mill-long (PRR+) numb isoform or miR-short numb isoform/numblike cassettes are simultaneously transferred by BP reaction into pDONR221. Then the regulable promoter(s) and miR-isoform cassettes are Multisite LR crossed into the modified pLenti6/EmGFP-bsd/R4R2-DES Tvector.

Multiple vectors can be generated in this manner comprising different combinations of synthetic oligonucleotides and transgene cassettes.

pLenti6/R4R2/V5-DEST vector sequence:
(SEQ ID NO: 1)
aatgtagtcttatgcaatactcttgtagtcttgcaacatggtaacgatgagttagcaacatgccttacaaggagagaaaaagca
ccgtgcatgccgattggtggaagtaaggtggtacgatcgtgccttattaggaaggcaacagacgggtctgacatggattgg
acgaaccactgaattgccgcattgcagagatattgtatttaagtgcctagctcgatacataaacgggtctctctggttagacca
gatctgagcctgggagctctctggctaactagggaacccactgcttaagcctcaataaagcttgccttgagtgcttcaagtagt
gtgtgcccgtctgltgtgtgactctggtaactagagatccctcagacccttttagtcagtgtggaaaatctctagcagtggcgc
ccgaacagggacttgaaagcgaaagggaaaccagaggagctctctcgacgcaggactcggcttgctgaagcgcgcacg
gcaagaggcgaggggcggcgactggtgagtacgccaaaaattttgactagcggaggctagaaggagagagatgggtgc
gagagcgtcagtattaagcgggggagaattagatcgcgatgggaaaaaattcggttaaggccagggggaaagaaaaaat
ataaattaaaacatatagtatgggcaagcagggagctagaacgattcgcagttaatcctggcctgttagaaacatcagaagg
ctgtagacaaatactgggacagctacaaccatcccttcagacaggatcagaagaacttagatcattatataatacagtagcaa
ccctctattgtgtgcatcaaaggatagagataaaagacaccaaggaagctttagacaagatagaggaagagcaaaacaaaa
gtaagaccaccgcacagcaagcggccgctgatcttcagacctggaggaggagatatgagggacaattggagaagtgaatt
atataaatataaagtagtaaaaattgaaccattaggagtagcacccaccaaggcaaagagaagagtggtgcagagagaaaa
aagagcagtgggaataggagctttgttccttgggltcttgggagcagcaggaagcactatgggcgcagcgtcaatgacgct
gacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacag
ctcctggggatttggggttgctctggaaaactcatttgcaccactgctgtgccttggaatgctagttggagtaataaatctctgg
aacagatttggaatcacacgacctggatggagtgggacagagaaattaacaattacacaagcttaatacactccttaattgaa
gaatcgcaaaaccagcaagaaaagaatgaacaagaattattggaattagataaatgggcaagtttgtggaattggtttaacat
aacaaattggctgtggtatataaaattattcataatgatagtaggaggcttggtaggtttaagaatagtttttgctgtactttctata
gtgaatagagttaggcagggatattcaccattatcgtttcagacccacctcccaaccccgaggggacccgacaggcccgaa
ggaatagaagaagaaggtggagagagagacagagacagatccattcgattagtgaacggatctcgacggtatcgatgtcg
acgttaacgctagtgatatcaactttgtatagaaaagttgaacgagaaacgtaaaatgatataaatatcaatatattaaattagat
tttgcataaaaaacagactacataatactgtaaaacacaacatatccagtcactatggcggccgcattaggcaccccaggcttt
acactttatgcttccggctcgtataatgtgtggattttgagttaggatccgtcgagattttcaggagctaaggaagctaaaatgg
agaaaaaaatcaclggatataccaccgttgatatatcccaatggcatcgtaaagaacattttgaggcatttcagtcagttgctca
atgtacctataaccagaccgttcagctggatattacggcctttttaaagaccgtaaagaaaaataagcacaagttttatccggc
ctttattcacattcttgcccgcctgatgaatgctcatccggaattccgtatggcaatgaaagacggtgagctggtgatatgggat
agtgttcacccttgttacaccgttttccatgagcaaactgaaacgttttcatcgctctggagtgaataccacgacgatttccggc
agtttctacacatatattcgcaagatgtggcgtgttacggtgaaaacctggcctatttccctaaagggtttattgagaatatgttttt
cgtctcagccaatccctgggtgagtttcaccagttttgatttaaacgtggccaatatggacaacttcttcgcccccgttttcacca
tgggcaaatattatacgcaaggcgacaaggtgctgatgccgctggcgatlcaggttcatcatgccgtttgtgatggcttccat
gtcggcagaatgcttaatgaattacaacagtactgcgatgagtggcagggcggggcgtaaagatctggatccggcttacta
aaagccagataacagtatgcgtatttgcgcgctgattttlgcggtataagaatatatactgatatgtatacccgaagtatgtcaa
aaagaggtatgctatgaagcagcgtattacagtgacagttgacagcgacagctatcagttgctcaaggcatatatgatgtcaa
tatctccggtctggtaagcacaaccatgcagaatgaagcccgtcgtctgcgtgccgaacgctggaaagcggaaaatcagg
aagggatggctgaggtcgcccggtttattgaaatgaacggctcttttgctgacgagaacagggactggtgaaatgcagttta
aggtttacacctataaaagagagagccgttatcgtctgtttgtggatgtacagagtgatattattgacacgcccgggcgacgg
atggtgatccccctggccagtgcacgtctgctgtcagataaagtctcccgtgaactttacccggtggtgcatatcggggatga
aagctggcgcatgatgaccaccgatatggccagtgtgccggtctccgttatcggggaagaagtggctgatctcagccaccg
cgaaaatgacatcaaaaacgccattaacctgatgttctggggaatataaatgtcaggctccgttatacacagccagtctgcag
gtcgaccatagtgactggatatgttgtgttttacagtattatgtagtctgttttttatgcaaaatctaatttaatatattgatatttatatc
attttacgtttctcgttcagctttcttgtacaaagtggttgatatccagcacagtggcggccgctcgagtctagagggcccgcg
gttcgaaggtaagcctatccctaaccctctcctcggtctcgattctacgcgtaccggttagtaatgagtttggaattaattctgtg
gaatgtgtgtcagttagggtgtggaaagtccccaggctccccagcaggcagaagtatgcaaagcatgcatctcaattagtca
gcaaccaggtgtggaaagtccccaggctccccagcaggcagaagtatgcaaagcatgcatctcaattagtcagcaaccata
gtcccgcccctaactccgcccatcccgcccctaactccgcccagttccgcccattctccgccccatggctgactaatttttttta
tttatgcagaggccgaggccgcctctgcctctgagctattccagaagtagtgaggaggcttttttaggaggcctaggcttttgca
aaaagctcccgggagcttgtatatccattttcggatctgatcagcacgtgttgacaattaatcatcggcatagtatatcggcata
gtataatacgacaciggtgaggaactaaaccatggccaagcctttgtctcaagaagaatccaccctcattgaaagagcaacg
gctacaatcaacagcatccccatctctgaagactacagcgtcgccagcgcagctctctctagcgacggccgcatcttcactg
gtgtcaatgtatatcattttactgggggaccttgtgcagaactcgtggtgctgggcactgctgctgctgcggcagctggcaac
ctgacttgtatcgtcgcgatcggaaatgagaacaggggcatcttgagcccctgcggacggtgccgacaggtgcttctcgat
ctgcatcctgggatcaaagccatagtgaaggacagtgatggacagccgacggcagttgggattcgtgaattgctgccctct
ggttatgtgtgggagggctaagcacaattcgagctcggtacctttaagaccaatgacttacaaggcagctgtagatcttagcc
actttttaaaagaaaaggggggactggaagggctaattcactcccaacgaagacaagatctgctttttgcttgtactgggtctc
tctggttagaccagatctgagcctgggagctctctggctaactagggaacccactgcttaagcctcaataaagcttgccttga
gtgcttcaagtagtgtgtgcccgtctgttgtgtgactctggtaactagagatccctcagacccttltagtcagtgtggaaaatctc
tagcagtagtagttcatgtcatcttattattcagtatttataacttgcaaagaaatgaatatcagagagtgagaggaacttgtttatt
gcagcttataatggttacaaataaagcaatagcatcacaaaUtcacaaataaagcatttttttcactgcattctagttgtggtttgt
ccaaactcatcaatgtatcttatcatgtctggctctagctatcccgcccctaactccgcccatcccgcccctaactccgcccagt
tccgcccattctccgccccatggctgactaattttttttatttatgcagaggccgaggccgcctcggcctctgagctattccaga
agtagtgaggaggcttttttggaggcctagggacgtacccaattcgccctatagtgagtcgtattacgcgcgctcactggccg
tcgttttacaacgtcgtgactgggaaaaccctggcgttacccaacttaatcgccttgcagcacatccccctttcgccagctggc
gtaatagcgaagaggcccgcaccgatcgcccttcccaacagttgcgcagcctgaatggcgaatgggacgcgccctgtag
cggcgcattaagcgcggcgggtgtggtggttacgcgcagcgtgaccgctacacttgccagcgccctagcgcccgctccttt
cgctttcttcccttcctttctcgccacgttcgccggctttccccgtcaagctctaaatcgggggctccctttagggttccgatttag
tgctttacggcacctcgaccccaaaaaacttgattagggtgatggttcacgtagtgggccatcgccctgatagacggtttttcg
ccctttgacgttggagtccacgttctttaatagtggactcttgttccaaactggaacaacactcaaccctatctcggtctattctttt
gatttataagggattttgccgatttcggcctattggttaaaaaatgagctgatttaacaaaaatttaacgcgaattttaacaaaata
ttaacgcttacaatttaggtggcacttttcggggaaatgtgcgcggaacccctatttgtttatttttctaaatacattcaaatatgtat
ccgctcatgagacaataaccctgataaatgcttcaataatattgaaaaaggaagagtatgagtattcaacatttccgtgtcgcc
cttattcccttttttgcggcattttgccttcctgtttttgctcacccagaaacgctggtgaaagtaaaagatgctgaagatcagttg
ggtgcacgagtgggttacatcgaactggatctcaacagcggtaagatccttgagagttttcgccccgaagaacgttttccaat
gatgagcacttttaaagtlctgctatgtggcgcggtattatcccgtattgacgccgggcaagagcaactcggtcgccgcatac
actattctcagaatgacttggttgagtactcaccagtcacagaaaagcatcttacggatggcatgacagtaagagaattatgca
gtgctgccataaccatgagtgataacactgcggccaacttacttctgacaacgatcggaggaccgaaggagctaaccgcttt
tttgcacaacatgggggatcatgtaactcgccttgatcgttgggaaccggagctgaatgaagccataccaaacgacgagcg
tgacaccacgatgcctgtagcaatggcaacaacgttgcgcaaactattaactggcgaactacttactctagcttcccggcaac
aattaatagactggatggaggcggataaagttgcaggaccacttctgcgctcggcccttccggctggctggtttattgctgat
aaatctggagccggtgagcgtgggtctcgcggtatcattgcagcactggggccagatggtaagccctcccgtatcgtagtta
tctacacgacggggagtcaggcaactatggatgaacgaaatagacagatcgctgagataggtgcctcactgattaagcatt
ggtaactgtcagaccaagtttactcatatatactttagattgatttaaaacttcatttttaatttaaaaggatctaggtgaagatcctt
tttgataatctcatgaccaaaatcccttaacgtgagttttcgttccactgagcgtcagaccccgtagaaaagatcaaaggatctt
cttgagatcctttttttctgcgcgtaatctgctgcttgcaaacaaaaaaaccaccgctaccagcggtggtttgtttgccggatca
agagctaccaactctttttccgaaggtaactggcttcagcagagcgcagataccaaatactgttcttctagtgtagccgtagtta
ggccaccacttcaagaactctgtagcaccgcctacatacctcgctctgctaatcctgttaccagtggctgctgccagtggcga
taagtcgtgtcttaccgggttggactaagacgatagttaccggataaggcgcagcggtcgggctgaacggggggttcgtgc
acacagcccagcttggagcgaacgacctacaccgaactgagatacctacagcgtgagctatgagaaagcgccacgcttcc
cgaagggagaaaggcggacaggtatccggtaagcggcagggtcggaacaggagagcgcacgagggagcttccaggg
ggaaacgcctggtatctttatagtcctgtcgggtttcgccacctctgacttgagcgtcgatttttgtgatgctcgtcaggggggc
ggagcctatggaaaaacgccagcaacgcggcctttttacggttcctggccttttgctggccttttgctcacatgttctttcctgc
gttatcccctgattctgtggataaccgtattaccgcctttgagtgagctgataccgctcgccgcagccgaacgaccgagcgc
agcgagtcagtgagcgaggaagcggaagagcgcccaatacgcaaaccgcctctccccgcgcgttggccgattcattaat
gcagctggcacgacaggtttcccgactggaaagcgggcagtgagcgcaacgcaattaatgtgagttagctcactcattagg
caccccaggctttacactttatgcttccggctcgtatgttgtgtggaattgtgagcggataacaatttcacacaggaaacagcta
tgaccatgattacgccaagcgcgcaattaaccctcactaaagggaacaaaagctggagctgcaagctt

### Example 4: Additional Methods for Generation of Therapeutic Vector(s)

“Packaging cell lines” derived from human and/or animal fibroblast cell lines result from transfecting or infecting normal cell lines with viral gag, pol, and env structural genes. On the other hand, packaging cell lines produce RNA devoid of the psi sequence, so that the viral particles produced from packaging cell do not contain the gag, pol, or env genes. Once the therapeutic vector's DNA containing the psi sequence (along with the therapeutic gene) is introduced into the packaging cell, by means of transfection or infection, the packaging cell may produce virions capable of transmitting the therapeutic RNA to the final target cell (e.g. a CD4+ cell).

The “infective range” of the therapeutic vector(s) is determined by the packaging cell line. A number of packaging cell lines are available for production of virus suitable for infecting a broad range of human cell types. These packaging cell lines are nevertheless generally capable of encapsidating viral vectors derived from viruses that in nature usually infect different animal species. For example, vectors derived from SIV or MMLV can be packaged by GP 120 encapsidating cell lines.

An example protocol for producing a therapeutic viral supernatant is provided as follows:

1. Twenty micrograms of retrovirus vector are mixed with 2-3 micrograms of viral DNA containing the selectable marker gene (e.g. antibiotic resistance gene) by gentle tapping in 0.8-1 milliliter of Ilepes buffered saline (pH=7.05) in a 1.5 ml plastic tube.

2. Seventy microliters of 2M CaCl are added to the mixture by repeated gentle tapping.

3. When a blue precipitate first begins to appear within the tube, the product should be gently applied to a 30% confluent layer of packaging cells (from any number of commercial vendors). The DNA mixture should be applied only after first removing the medium from the packaging cells.

4. The packaging cells are set to incubate for 20-30 minutes at room temperature (25 degrees Celsius) before transferring them back to an incubator at 36-38 degrees Celsius for 3.5 hours.

5. Add 3.5-4 milliliters of Hepes buffered saline containing 15% glycerol for 3 minutes then wash cell with Dulbecco's Modified Eagle's Medium (DMEM)+! 0% FBS×2.

6. Add back DMKM+10% FBS, and incubate cells for 20 hours at 37 degrees Celsius.

7. Remove and filter medium containing therapeutic viral particles.

Excess viral supernatant is immediately stored or concentrated and stored at −80 degrees Celsius). Supernatant may stored with 5-8 micrograms of polybrene to increase the efficiency of target cell infection. Otherwise polybrene may be excluded or added just before infection.

8. Stable producer lines can be established by splitting packaging cell lines 1 to 20, or 1 to 40 and subsequently incubating these cells for up to 10 days (changing medium every three days) in medium containing selective drugs (e.g. certain antibiotics corresponding to transfected resistance genes).

9. After 10 days isolated colonies are picked, grown-up aliquoted and frozen for storage.

Assay of Retrovirus Infectivity/Titration is achieved by application of a defined volume of viral supernatant to a layer of confluent “test” cells such as NIII 3T3 cells plated at 20% confluence. After 2-3 cell division times (24-36 hours for NIH 3T3 cells) colonies of “test” cells incubated at 37 degrees in antibiotic-containing medium are counted. The supernatant's titer are estimated from these colony counts by the following formula:

Colony Forming Units/ml=colonies identified×0.5(split factor)/volume of virus(ml)

The accuracy of this estimate is increased by testing large volumes of supernatant over many plates of “test” cells.

Application of the therapeutic viral supernatant to target cells may be accomplished by various means appropriate to the clinical situation.

### Example 5: Growth Medium for Selected Cells

Selected cells can be expanded/grown in Dulbecco's modified Minimal Essential Medium (DMEM) supplemented with glutamine, beta.-mercaptoethanol, 10% (by volume) horse serum, and human recombinant Leukemia Inhibitory Factor (LIF). LIF replaces the need for maintaining selected cells on feeder layers of cells, (which may also be employed) and is essential for maintaining selected cells in an undifferentiated, multipotent, or pluripotent state, such cells can be maintained in Dulbecco's modified Minimal Essential Medium (DMEM) supplemented with glutamine, beta.-mercaptoethanol, 10% (by volume) horse serum, and human recombinant Leukemia Inhibitory Factor (LIF). The LIF replaces the need for maintaining cells on feeder layers of cells, (which may also be employed) and is essential for maintaining cells in an undifferentiated state (per U.S. Pat. No. 6,432,711).

In order to initiate the differentiation of the selected cells into neuronal cells, the cells are trypsinized and washed free of LIF, and placed in DMEM supplemented with 10% fetal bovine serum (FBS). After resuspension in DMEM and 10% FBS, IX105 cells are plated in 5 ml DMEM, 10% FBS, 0.5 microM retinoic acid in a 60 mm Fisher bacteriological grade Petri dishes, where the cells are expected to form small aggregates. Aggregation aids in proper cell differentiation. High efficiency transfection with appropriate neuronal transcription factors can occur before or after plating in DMEM, FBS, and retinoic acid. (See U.S. Pat. Nos. 6,432,711 and 5,453,357 for additional details).

### Example 6: Hla Matching

Selected cells (e.g. umbilical cord blood or cells from any other suitable source and/or their progeny), can be screened, genetically-modified (optional), expanded, and induced to begin differentiating into the desired cell type(s) (optional). The cells are then transplanted according to standard stem cell transplantation protocols. In certain instances, cells may be transplanted into patients without HLA matching.

### Example 7

In some rare instance, it may be appropriate to introduce transgene encoding vectors into patients in order to stimulate or inhibit cellular division or cellular differentiation, in vivo.

### Example 8: Genetic Modification of Selected Cells

In vitro genetic modification of exogenous cells or patient's endogenous cells can be performed according to any published or unpublished method known to the art (e.g. U.S. Pat. Nos. 6,432,711, 5,593,875, 5,783,566, 5,928,944, 5,910,488, 5,824,547, etc.) or by other generally accepted means. Suitable methods for transforming host cells can be found in Sambrook et al. (Molecular Cloning: A Laboratory Manual, 2nd Edition, Cold Spring Harbor Laboratory press (1989)), and other laboratory textbooks.

Successfully transfected/contacted cells are identified by selection protocols involving markers such as antibiotic resistance genes in addition to RNA expression assays and morphological analyses. Clones from successfully transfected/contacted cells, expressing the appropriate exogenous DNA at appropriate levels, can be preserved as cell lines by cryopreservation (utilizing any appropriate method of cryopreservation known to the art).

Selectable markers (e.g., antibiotics resistance genes) may include those conferring resistance to drugs, such as G418, hygromycin and methotrexate. Cells containing the gene of interest can be identified by drug selection where cells that have incorporated the selectable marker gene survive, and others die.

The current invention discloses the selection of genetically-modified cells as “selected cells” of the invention. The term genetic modification refers to alteration of the cellular genotype by introducing natural or synthetic nucleic acids into selected cells and/or their progeny or immortalized cell lines and/or their progeny by any means known to the art. Alternatively culture conditions that induce permanent changes in gene expression patterns are considered herein to represent genetic modification. Modification of stem cells, whether they be derived from the host brain, endogenous donor sources, exogenous donor sources, or cell lines, represents a feasible approach to the treatment of certain human diseases, especially those of the human nervous system.

Genetic modifications covered by this disclosure include, but are not limited to: genetic modifications performed in vivo; modifications that alter the activity or amount of metabolic enzymes expressed by endogenous or exogenous selected cells and/or their progeny; modifications which alter the activity, amount, or antigenicity of cellular proteins; modifications which alter the activity or amount of proteins involved in signal transduction pathways; modifications which alter HLA type; modifications which alter cellular differentiation; modifications which alter neoplastic potential; modifications which alter cellular differentiation; modifications which alter the amount or activity of structural proteins; modifications which alter the amount or activity of membrane associated proteins (structural or enzymatic); modifications which alter the activity or amount of proteins involved in DNA repair and chromosome maintenance; modifications which alter the activity or amount of proteins involved in cellular transport; modifications which alter the activity or amount of enzymes; modifications which alter the activity or amount of proteins involved in synapse formation and maintenance; modifications which alter the activity or amount of proteins involved in neurite outgrowth or axon outgrowth and formation; modifications altering the amount or activity of antioxidant producing enzymes within the cell; modifications which lead to altered post-translational modification of cellular proteins; modifications which alter the activity or amount of proteins involved in other aspects of cellular repair, and alterations which increase the lifespan of the cell (such as production of telomerase). Such proteins as those mentioned above may be encoded for by DNA or RNA derived from the human genome or other animal, plant, viral, or bacterial genomes. This invention also covers sequences designed de novo.

In addition, this invention relates to the in situ, genetic modification of selected cells and/or their progeny cells for the treatment of disease. Endogenous stem cells may be modified in situ by direct injection or application of DNA or RNA vectors, including viruses, retroviruses, liposomes, etc, into the substance of the tissue or into the appropriate portion of the ventricular system of the brain. Since 1992, we have modified thousands of stem/progenitor cells and many thousand progeny cells in this manner. Our data shows that this manner of modifying progenitor cells results in a tremendous variety of modified cell types throughout the nervous system, and has never resulted in adverse effects.

### Example 9: Introduction of Genetic Vectors into the Host

In a preferred embodiment, endogenous cells are transfected/contacted with vectors such as those described herein in vivo by introduction of the therapeutic vector(s) into the host blood, tissues, nervous system, bone marrow, etc. The greatest benefit may be achieved by modifying a large number of endogenous target cells. This may be accomplished by using an appropriately-sized, catheter-like device, or needle to inject the therapeutic vector(s) into the venous or arterial circulation, into a specific tissue, such as muscle tissue, or into the nervous system. In a preferred embodiment, the virus is pseudotyped with VSV-G envelope glycoprotein and native HIV-I env proteins.

### Example 10: Injection into the Nervous System

Transplantation of selected cells (from either the growth or differentiation media) into the fetal nervous system or genetic modification of endogenous fetal cells utilizing genetic vectors may be accomplished in the following manner Under sterile conditions, the uterus and fetuses are visualized by ultrasound or other radiological guidance. Alternatively the uterus may be exposed surgically in order to facilitate direct identification of fetal skull landmarks. Selected cells can then be introduced by injection (using an appropriately-sized catheter or needle) into the ventricular system, germinal zone(s), or into the substance of the nervous system. Injections may be performed in certain instances, through the mother's abdominal wall, the uterine wall and fetal membranes into the fetus. The accuracy of the injection is monitored by direct observation, ultrasound, contrast, or radiological isotope based methods, or by any other means of radiological guidance known to the art.

Under appropriate sterile conditions, direct identification of fetal skull landmarks is accomplished visually as well as by physical inspection and palpation coupled with stereotaxic and radiologic guidance. Following cell culture, appropriate amounts of the selected or differentiating cells can then be introduced by injection or other means into the ventricular system, germinal zones, or into the substance of the nervous system. The accuracy of the injection may be monitored by direct observation, ultrasound, or other radiological guidance.

In certain, neurological diseases of the adult nervous system, such as Huntington's disease and Parkinson's disease, cells of a specific portion of the brain are selectively affected. In the case of Parkinson's disease, it is the dopaminergic cells of the substantia nigra. In such regionally-specific diseases affecting adults, localized transplantation of cells may be accomplished by radiologically-guided transplantation of differentiating cells under sterile conditions. Radiologic guidance may include the use of CT and/or MRI, and may take advantage contrast or isotope based techniques to monitor injected materials.

In certain neurologic diseases, such as some metabolic storage disorders, cells are affected across diverse regions of the nervous system, and the greatest benefit may be achieved by genetically-modifying endogenous cells or introducing selected cells of the present invention (either from the growth culture media or the differentiating medium) into the tissue in large numbers in a diffuse manner. In the nervous system, these diseases may be best approached by intraventricular injections (using an appropriately-sized, catheter-like device, or needle) (especially at early stages of development) which allows diffuse endogenous cell modification or diffuse engraflment of selected cells isolated from the growth and/or differentiation media. Nevertheless, injection of the cells into the circulatory system for the same purpose is also covered. However, with regard to any disorder affecting multiple organs or the body diffusely (e.g. lysosomal storage disorders, hemoglobinpathies, muscular dystrophy), the cells isolated from the growth and/or differentiation media may also be preferentially introduced directly into the circulation and/or visceral organs, such as the liver, kidney, gut, spleen, adrenal glands, pancreas, lungs, and thymus using endoscopic guidance and any appropriately-sized, catheter-like device, allowing diffuse engraftment of the cells throughout the body, as well as specific introduction and infiltration of the cells into the selected organs.

### Example 11: Delivery of Cells by Injection in to the Circulatory Stream and Organs

Diseases of one organ system may be treatable with genetically modified cells from a separate organ system. Also, in some instances, it may become apparent that the selected cells may integrate and differentiate on their own, in vivo, in sufficient numbers if they are injected into blood stream either arterial, venous or hepatic, after culturing in the growth and/or differentiation media. This approach is covered by the present invention. The treatment of diffuse muscle (e.g. muscular dystrophies), organ, tissue, or blood disorders (e.g. Hereditary Spherocytosis, Sickle cell anemia, other hemoglobinopathies, etc,) may, for instance, involve the injection of cells isolated from the growth media or differentiating media into the patient, especially the patient's circulation. This approach is also believed to ameliorate ischemic injuries such as myocardial infarction, stroke, etc., as well as traumatic injuries to brain and other tissues. Injection of such cells produced by the current invention, directly into the circulation, by needle or catheter, so that the cells are enabled to “home” to the bone marrow, muscle, kidneys, lungs, and/or any other other organ system, as well as injection directly into the bone marrow space is suitable for the practice of the present invention. Likewise injection of the cells directly into a lesion site with or without radiologic, ultrasonic or fluoroscopic guidance is also suitable for the practice of the present invention.

Methods of isolating selected cells useful in the present invention include those described by Zhao et al., 2006.

In a preferred embodiment, genetic vectors encoding numblike and/or numb isoforms comprise regulable promoters operably linked to the Numb or numblike transgenes.

In another preferred embodiment, the mode of transfection may be selected from those modes of transfection/contacting that provide for transient rather than permanent expression of the numblike and numb isoforms.

### Example 12

Example Genetic Modifications It is believed that hundreds of diseases and clinical conditions are able to be treated and/or ameliorated by the methods of the present invention including, but in no way limited to Canavan's disease (ASP); Tay-Sach's disease (HEXA); Lesch-Nyhan syndrome (HRPT); Huntington's disease(HTT); Sly syndrome; type A and type B Niemann Pick disease;

Sandhoff s disease (HEXB); Fabry's disease (GLA); type C Niemann-Pick disease(NPC1); Gaucher's disease (GBA); Parkinson's disease(PARK2, etc.); Von Hippel Lindau's disease, Sickle cell anemia (HBB) and other thalassemias as well as similar diseases. These transgenes may represent the coding region or portions of the coding region of the normal genes.

It is to be understood, however, that the scope of the present invention is not to be limited to the specific embodiments and examples described above. The invention may be practiced other than as particularly described and still be within the scope of the accompanying claims.

### Example 13

An example sequence for a vector capable of rendering cells pluripotent and expressing a long Numb isoform, Oct-4, Sox-2, and EmGFP nucleic acid sequences under the control of tetracycline-sensitive promoters is:

(SEQ ID NO: 2)
aatgtagtcttatgcaatactcttgtagtcttgcaacatggtaacgatgagttagcaacatgccttacaaggagagaaaaagca
ccgtgcatgccgattggtggaagtaaggtggtacgatcgtgccttattaggaaggcaacagacgggtctgacatggattgg
acgaaccactgaattgccgcattgcagagatattgtatttaagtgcctagctcgatacataaacgggtctctctggttagacca
gatctgagcctgggagctctctggctaactagggaacccactgcttaagcctcaataaagcttgccttgagtgcttcaagtagt
gtgtgcccgtctgttgtgtgactciggtaactagagatccctcagacccttttagtcagtgtggaaaatctctagcagtggcgc
ccgaacagggacttgaaagcgaaagggaaaccagaggagctctctcgacgcaggactcggcttgctgaagcgcgcacg
gcaagaggcgaggggcggcgactggtgagtacgccaaaaattttgactagcggaggctagaaggagagagatgggtgc
gagagcgtcagtattaagcgggggagaattagatcgcgatgggaaaaaattcggttaaggccagggggaaagaaaaaat
ataaattaaaacatatagtatgggcaagcagggagctagaacgattcgcagttaatcctggcctgttagaaacatcagaagg
ctgtagacaaatactgggacagctacaaccatcccttcagacaggatcagaagaacttagatcattatataatacagtagcaa
ccctctattgtgtgcatcaaaggatagagataaaagacaccaaggaagctttagacaagatagaggaagagcaaaacaaaa
gtaagaccaccgcacagcaagcggccgctgatcttcagacctggaggaggagatatgagggacaattggagaagtgaatt
atataaatataaagtagtaaaaattgaaccattaggagtagcacccaccaaggcaaagagaagagtggtgcagagagaaaa
aagagcagtgggaataggagctttgttccttgggttcttgggagcagcaggaagcactatgggcgcagcgtcaatgacgct
gacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacag
ctcctggggatttggggttgctctggaaaactcatttgcaccactgctgtgccttggaatgctagttggagtaataaatctctgg
aacagatttggaatcacacgacctggatggagtgggacagagaaattaacaattacacaagcttaatacactccttaattgaa
gaatcgcaaaaccagcaagaaaagaatgaacaagaattattggaattagataaatgggcaagtttgtggaattggtttaacat
aacaaattggctgtggtatataaaattattcataatgatagtaggaggcttggtaggtttaagaatagtttttgctgtactttctata
gtgaatagagttaggcagggatattcaccattatcgtttcagacccacctcccaaccccgaggggacccgacaggcccgaa
ggaatagaagaagaaggtggagagagagacagagacagatccattcgattagtgaacggatctcgacggtatcgatgtcg
acgttaacgctagtgatatcaactttgtatagaaaagttgaacgagaaacgtaaaatgatataaatatcaatatattaaattagat
tttgcataaaaaacagactacataatactgtaaaacacaacatatccagtcactatgggacggatcgggagatctcccgatcc
cctatggtgcactctcagtacaatctgctctgatgccgcatagttaagccagtatctgctccctgcttgtgtgttggaggtcgct
gagtagtgcgcgagcaaaatttaagctacaacaaggcaaggcttgaccgacaattgcatgaagaatctgcttagggttagg
cgttttgcgctgcttcgcgatgtacgggccagatatacgcgttgacattgattattgactagttattaatagtaatcaattacggg
gtcattagttcatagcccatatatggagttccgcgttacataacttacggtaaatggcccgcctggctgaccgcccaacgacc
cccgcccattgacgtcaataatgacgtatgttcccatagtaacgccaatagggactttccattgacgtcaatgggtggagtattt
acggtaaactgcccacttggcagtacatcaagtgtatcatatgccaagtacgccccctattgacgtcaatgacggtaaatggc
ccgcctggcattatgcccagtacatgaccttatgggactttcctacttggcagtacatctacgtattagtcatcgctattaccatg
gtgatgcggttttggcagtacatcaatgggcgtggatagcggtttgactcacggggatttccaagtctccaccccattgacgt
caatgggagtttgttttggaaccaaaatcaacgggactttccaaaatgtcgtaacaactccgccccattgacgcaaatgggcg
gtaggcgtgtacggtgggaggtctatataagcagagctctccctatcagtgatagagatctccctatcagtgatagagatcgt
cgacgagctcgtttagtgaaccgtcagatcgcctggagacgccatccacgctgttttgacctccatagaagacaccgggac
cgatccagcctccggactctagcgtttaaacttaagcttaccatgccttcgcaagccctcatttcaccaggcccccggcttgg
ggcgccttccttccccatggcgggacacctggcttcggatttcgccttctcgccccctccaggtggtggaggtgatgggcca
ggggggccggagccgggctgggttgatcctcggacctggctaagcttccaaggccctcctggagggccaggaatcggg
ccgggggttgggccaggctctgaggtgtgggggattcccccatgccccccgccgtatgagttctgtggggggatggcgta
ctgtgggccccaggttggagtggggctagtgccccaaggcggcttggagacctctcagcctgagggcgaagcaggagtc
ggggtggagagcaactccgatggggcctccccggagccctgcaccgtcacccctggtgccgtgaagctggagaaggag
aagctggagcaaaacccggaggagtcccaggacatcaaagctctgcagaaagaactcgagcaatttgccaagctcctgaa
gcagaagaggatcaccctgggatatacacaggccgatgtggggctcaccctgggggttctatttgggaaggtattcagcca
aacgaccatctgccgctttgaggctctgcagcttagcttcaagaacatgtgtaagctgcggcccttgctgcagaagtgggtg
gaggaagctgacaacaatgaaaatcttcaggagatatgcaaagcagaaaccctcgtgcaggcccgaaagagaaagcgaa
ccagtatcgagaaccgagtgagaggcaacctggagaatttgttcctgcagtgcccgaaacccacactgcagcagatcagc
cacatcgcccagcagcttgggctcgagaaggatgtggtccgagtgtggttctgtaaccggcgccagaagggcaagcgatc
aagcagcgactatgcacaacgagaggattttgaggctgctgggtctcctttctcagggggaccagtgtcctttcctctggccc
cagggccccattttggtaccccaggctatgggagccctcacttcactgcactgtactcctcggtccctttccctgagggggaa
gcattccccctgtctccgtcaccactctgggctctcccatgcattcaaactgaggtgcctgcccttctaggaatgggggaca
gggggaggggaggagctagggaaagaaaacctggagtttgtgccagggtttttgggattaagttcttcattcactaaggaag
gaattgggaacacaaagggtgggggcaggggagtttggggcaactggttggagggaaggtgaagttcaatgatgctcttg
attttaatcccacatcatgtatcacttttttcttaaataaagaagcctgggacacagtagatagacacacttaaaaaaaaaaacct
cgactgtgccttctagttgccagccatctgttgtttgcccctcccccgtgccttccttgaccctggaaggtgccactcccactgt
cctttcctaataaaatgaggaaattgcatcgcattgtctgagtaggtgtcattctattctggggggtggggtggggcaggaca
gcaagggggaggattgggaagacaatagcaggcatgctggggatgcggtgggctctatgggacggatcgggagatctc
ccgatcccctatggtgcactctcagtacaatcttgctctgatgccgcatagttaagccagtatctgctccctgcttgtgtgttgga
ggtcgctgagtagtgcgcgagcaaaatttaagctacaacaaggcaaggcttgaccgacaattgcatgaagaatctgcttag
ggttaggcgttttgcgctgcttcgcgatgtacgggccagatatacgcgttgacattgattattgactagttattaatagtaatcaa
ttacggggtcattagttcatagcccatatatggagttccgcgttacataacttacggtaaatggcccgcctggctgaccgccca
acgacccccgcccattgacgtcaataatgacgtatgttcccatagtaacgccaatagggactttccattgacgtcaatgggtg
gagtatttacggtaaactgcccacttggcagtacatcaagtgtatcatatgccaagtacgccccctattgacgtcaatgacggt
aaatggcccgcctggcattatgcccagtacatgaccttatgggactttcctacttggcagtacatctacgtattagtcatcgctat
taccatggtgatgcggttttggcagtacatcaatgggcgtggatagcggtttgactcacggggatttccaagtctccaccccat
tgacgtcaatgggagtttgttttggaaccaaaatcaacgggactttccaaaatgtcgtaacaactccgccccattgacgcaaat
gggcggtaggcgtgtacggtgggaggtctatataagcagagctctccctatcagtgatagagatctccctatcagtgataga
gatcgtcgacgagctcgtttagtgaaccgtcagatcgcctggagacgccatccacgctgttttgacctccatagaagacacc
gggaccgatccagcctccggactctagcgttlaaacttaagcttaccatgctattaacttgttcaaaaaagtatcaggagttgtc
aaggcagagaagagagtgtttgcaaaagggggaaagtagtttgctgcctctttaagactaggactgagagaaagaagagg
agagagaaagaaagggagagaagtttgagccccaggcttaagcctttccaaaaaataataataacaatcatcggcggcgg
caggatcggccagaggaggagggaagcgctttttttgatcctgattccagtttgcctctctctttttttcccccaaattattcttcg
cctgattttcctcgcggagccctgcgctcccgacacccccgcccgcctcccctcctcctctccccccgcccgcgggccccc
caaagtcccggccgggccgagggtcggcggccgccggcgggccgggcccgcgcacagcgcccgcatgtacaacatg
atggagacggagctgaagccgccgggcccgcagcaaacttcggggggcggcggcggcaactccaccgcggcggcgg
ccggcggcaaccagaaaaacagcccggaccgcgtcaagcggcccatgaatgccttcatggtgtggtcccgcgggcagc
ggcgcaagatggcccaggagaaccccaagatgcacaactcggagatcagcaagcgcctgggcgccgagtggaaactttt
gtcggagacggagaagcggccgttcatcgacgaggctaagcggctgcgagcgctgcacatgaaggagcacccggatta
taaataccggccccggcggaaaaccaagacgctcatgaagaaggataagtacacgctgcccggcgggctgctggccccc
ggcggcaatagcatggcgagcggggtcggggtgggcgccggcctgggcgcgggcgtgaaccagcgcatggacagtta
cgcgcacatgaacggctggagcaacggcagctacagcatgatgcaggaccagctgggctacccgcagcacccgggcct
caatgcgcacggcgcagcgcagatgcagcccatgcaccgctacgacgtgagcgccctgcagtacaactccatgaccagc
tcgcagacctacatgaacggctcgcccacctacagcatgtcctactcgcagcagggcacccctggcatggctcttggctcc
atgggttcggtggtcaagtccgaggccagctccagcccccctgtggttacctcttcctcccactccagggcgccctgccag
gccggggacctccgggacatgatcagcatgtatctccccggcgccgaggtgccggaacccgccgcccccagcagacttc
acatgtcccagcactaccagagcggcccggtgcccggcacggccattaacggcacactgcccctctcacacatgtgaggg
ccggacagcgaactggaggggggagaaattttcaaagaaaaacgagggaaatgggaggggtgcaaaagaggagagta
agaaacagcatggagaaaacccggtacgctcaaaaagaaaaaggaaaaaaaaaaatcccatcacccacagcaaatgaca
gctgcaaaagagaacaccaatcccatccacactcacgcaaaaaccgcgatgccgacaagaaaacttttatgagagagatc
ctggacttctttttgggggactatttttgtacagagaaaacctggggagggtggggagggcgggggaatggaccttgtatag
atctggaggaaagaaagctacgaaaaactttttaaaagttctagtggtacggtaggagctttgcaggaagtttgcaaaagtctt
taccaataatatttagagctagtctccaagcgacgaaaaaaatgttttaatatttgcaagcaacttttgtacagtatttatcgagat
aaacatggcaatcaaaatgtccattgtttataagctgagaatttgccaataUtttcaaggagaggcttcttgctgaattttgattct
gcagctgaaatttaggacagttgcaaacgtgaaaagaagaaaattattcaaatttggacattttaattgtttaaaaattgtacaaa
aggaaaaaattagaataagtactggcgaaccatctctgtggtcttgtttaaaaagggcaaaagttttagactgtactaaattttat
aacttactgttaaaagcaaaaatggccatgcaggttgacaccgttggtaatttataatagcttttgttcgatcccaactttccatttt
gttcagataaaaaaaaccatgaaattactgtgtttgaaatattttcttatggtttgtaatatttctgtaaatttaUgtgatattttaagg
ttttcccccctttattttccgtagttgtaUttaaaagattcggctctgtattatttgaatcagtctgccgagaatccatgtatatatttg
aactaatatcatccttataacaggtacattttcaacttaagtattttctccattatgcacagtttgagataaataaatttttgaaatatg
gacactgaaaaaaaaaaaaaaaaaacctcgactgtgccttctagttgccagccatctgttgtttgcccctcccccgtgccttcc
ttgaccctggaaggtgccactcccactgtcctttcctaataaaatgaggaaattgcatcgcattgtctgagtaggtgtcattctat
tctggggggtggggtggggcaggacagcaagggggaggattgggaagacaatagcaggcatgctggggatgcggtgg
gctctatgggacggatcgggagatctcccgatcccctatggtgcactctcagtacaatctgctctgatgccgcatagttaagc
cagtatctgctccctgcttgtgtgttggaggtcgctgagtagtgcgcgagcaaaatttaagctacaacaaggcaaggcttgac
cgacaattgcatgaagaatctgcttagggttaggcgttttgcgctgcttcgcgatgtacgggccagatatacgcgttgacattg
attattgactagttattaatagtaatcaattacggggtcattagttcatagcccatatatggagttccgcgttacataacttacggta
aatggcccgcctggctgaccgcccaacgacccccgcccattgacgtcaataatgacgtatgttcccatagtaacgccaatag
ggactttccattgacgtcaatgggtggagtatttacggtaaactgcccacttggcagtacatcaagtgtatcatatgccaagta
cgccccctattgacgtcaatgacggtaaatggcccgcctggcattatgcccagtacatgaccttatgggactttcctacttggc
agtacatctacgtattagtcatcgctattaccatggtgatgcggttttggcagtacatcaatgggcgtggatagcggtttgactc
acggggatttccaagtctccaccccattgacgtcaatgggagtttgttttggaaccaaaatcaacgggactttccaaaatgtcg
taacaactccgccccattgacgcaaatgggcggtaggcgtgtacggtgggaggtctatataagcagagctctccctatcagt
gatagagatctccctatcagtgatagagatcgtcgacgagctcgtttagtgaaccgtcagatcgcctggagacgccatccac
gctgttttgacctccatagaagacaccgggaccgatccagcctccggactctagcgtttaaacttaagcttaccatggttgtca
tgggggaggtggtggcgcttggtggccactggcggccgaggtagaggcagtggcgcttgagttggtcgggggcagcgg
cagatttgaggcttaagcaacttcttccggggaagagtgccagtgcagccactgttacaattcaagatcttgatctatatccata
gattggaatattggtgggccagcaatcctcagacgcctcacttaggacaaatgaggaaactgaggcttggtgaagttacgaa
acttgtccaaaatcacacaacttgtaaagggcacagccaagaUcagagccaggctgtaaaaattaaaatgaacaaattacg
gcaaagttttaggagaaagaaggatgtttatgttccagaggccagtcgtccacatcagtggcagacagatgaagaaggcgtt
cgcaccggaaaatgtagcttcccggttaagtaccttggccatgtagaagttgatgaatcaagaggaatgcacatctgtgaag
atgctgtaaaaagattgaaagctgaaaggaagttcttcaaaggcttctttggaaaaactggaaagaaagcagttaaagcagtt
ctgtgggtctcagcagatggactcagagttgtggatgaaaaaactaaggacctcatagttgaccagacgatagagaaagttt
cUtctgtgccccagacaggaactttgatagagccttttcttacatatgccgtgatggcaccactcgtcgctggatctgtcactg
cttcatggctgtcaaggacacaggtgaaaggttgagccatgcagtaggctgtgcttttgcagcctgtttagagcgcaagcag
aagcgggagaaggaatgtggagtgactgctacttttgatgctagtcggaccacttttacaagagaaggatcattccgtgtcac
aacagccactgaacaagcagaaagagaggagatcatgaaacaaatgcaagatgccaagaaagctgaaacagataagata
gtcgttggttcatcagttgcccctggcaacactgccccatccccatcctctcccacctctcctacttctgatgccacgacctctct
ggagatgaacaatcctcatgccatcccacgccggcatgctccaattgaacagcttgctcgccaaggctctttccgaggttttc
ctgctcttagccagaagatgtcaccctttaaacgccaactatccctacgcatcaatgagltgccttccactatgcagaggaaga
ctgatttccccattaaaaatgcagtgccagaagtagaaggggaggcagagagcatcagctccctgtgctcacagatcacca
atgccttcagcacacctgaggaccccttctcatctgctccgatgaccaaaccagtgacagtggtggcaccacaatctcctacc
ttccaagctaatggcactgactcagccttccatgtgcttgctaagccagcccatactgctctagcacccgtagcaatgcctgtg
cgtgaaaccaacccttgggcccatgcccctgatgctgclaacaaggaaattgcagccacatgtlcggggaccgagtggggt
caatcttctggtgctgcctctccaggtctcttccaggccggtcatagacgtactccctctgaggccgaccgatggttagaaga
ggtgtctaagagcgtccgggctcagcagccccaggcctcagctgctcctctgcagccagttctccagcctcctccacccact
gccatctcccagccagcatcacctttccaagggaatgcattcctcacctctcagcctgtgccagtgggtgtggtcccagccct
gcaaccagcctttgtocctgcccagtcctatcctgtggccaatggaatgccctatccagcccctaatgtgcctglgglgggca
tcactccctcccagatggtggccaacgtatttggcactgcaggccaccctcaggctgcccatccccatcagtcacccagcct
ggtcaggcagcagacattccctcactacgaggcaagcagtgctaccaccagtcccttctttaagcctcctgctcagcacctc
aacggttctgcagctttcaatggtgtagatgatggcaggttggcctcagcagacaggcatacagaggttcctacaggcacct
gcccagtggatccttttgaagcccagtgggctgcattagaaaataagtccaagcagcgtactaatccctcccctaccaaccct
ttctccagtgacttacagaagacgtttgaaattgaactttaagcaatcattatggctatgtatcttgtccataccagacagggagc
agggggtagcggtcaaaggagcaaaacagactttgtctcctgattagtactcttttcactaatcccaaaggtcccaaggaaca
agtccaggcccagagtactgtgaggggtgattttgaaagacatgggaaaaagcattcctagagaaaagctgccttgcaatta
ggctaaagaagtcaaggaaatgttgctttctgtactccctcttcccttacccccttacaaatctctggcaacagagaggcaaag
tatctgaacaagaatctatattccaagcacatttactgaaatgtaaaacacaacaggaagcaaagcaatctccctttgtttttcag
gccattcacctgcctcctgtcagtagtggcctgtattagagatcaagaagagtggtttgtgctcaggctggggaacagagag
gcacgctatgctgccagaattcccaggagggcatatcagcaactgcccagcagagctatattttgggggagaagttgagctt
ccatttlgagtaacagaataaatattatatatatcaaaagccaaaatctttatttttatgcatttagaatattttaaatagttctcagat
attaagaagttgtatgagttgtaagtaatcttgccaaaggtaaaggggctagttgtaagaaattgtacataagattgatttatcatt
gatgcctactgaaataaaaagaggaaaggctggaagctgcagacaggatccctagcttgttttctgtcagtcattcattgtaag
tagcacattgcaacaacaatcatgcttatgaccaatacagtcactaggttgtagtttUtttaaataaaggaaaagcagtattgtc
ctggttttaaacctatgatggaattctaatgtcaUattttaatggaatcaatcgaaatatgctctatagagaatatatcttttatatat
tgclgcagtttccttatgttaatcctttaacactaaggtaacatgacataatcataccatagaagggaacacaggttaccatattg
gtttgtaatatgggtcttggtgggttttgttttatcctttaaattttgttcccatgagttttgtggggatggggattctggttttattagct
ttgtgtgtgtcctcttcccccaaacccccttttggtgagaacatccccttgacagttgcagcctcttgacctcggataacaataa
gagagctcatctcatttttacttttgaacgttggccttacaatcaaatgtaagttatatatatttgtactgatgaaaatttataatctgc
tttaacaaaaataaatgttcatggtagaagcttttaaaaaaaaaaaaacctcgactgtgccttctagttgccagccatctgttgttt
gcccctcccccgtgccttccttgaccctggaaggtgccactcccactgtcctttcctaataaaatgaggaaattgcatcgcatt
gtctgagtaggtgtcattctattctggggggtggggtggggcaggacagcaagggggaggattgggaagacaatagcag
gcatgctggggatgcggtgggctctatgggacggatcgggagatctcccgatcccctatggtgcactctcagtacaatctgc
tctgatgccgcatagttaagccagtatctgctccctgcttgtgtgttggaggtcgctgagtagtgcgcgagcaaaatttaagct
acaacaaggcaaggcttgaccgacaattgcatgaagaatctgcttagggttaggcgttttgcgctgcttcgcgatgtacggg
ccagatatacgcgttgacattgattattgactagttattaatagtaatcaattacggggtcattagttcatagcccatatatggagt
tccgcgttacataacttacggtaaatggcccgcctggctgaccgcccaacgacccccgcccattgacgtcaataatgacgta
tgttcccatagtaacgccaatagggactttccattgacgtcaatgggtggagtatttacggtaaactgcccacttggcagtaca
tcaagtgtatcatatgccaagtacgccccctattgacgtcaatgacggtaaatggcccgcctggcattatgcccagtacatga
ccttatgggactttcctacttggcagtacatctacgtattagtcatcgctattaccatggtgatgcggttttggcagtacatcaatg
ggcgtggatagcggtttgactcacggggatttccaagtctccaccccattgacgtcaatgggagtttgttttggaaccaaaatc
aacgggactttccaaaatgtcgtaacaactccgccccattgacgcaaatgggcggtaggcgtgtacggtgggaggtctatat
aagcagagctctccctatcagtgatagagatctccctatcagtgatagagatcgtcgacgagctcgtttagtgaaccgtcaga
tcgcctggagacgccatccacgctgttttgacctccatagaagacaccgggaccgatccagcctccggactctagcgtttaa
acttaagcttaccatggtgagcaagggcgaggagctgttcaccggggtggtgcccatcctggtcgagctggacggcgacg
taaacggccacaagttcagcgtgtccggcgagggcgagggcgatgccacctacggcaagctgaccctgaagttcatctgc
accaccggcaagctgcccgtgccctggcccaccctcgtgaccaccttcacctacggcgtgcagtgcttcgcccgctacccc
gaccacatgaagcagcacgacttcttcaagtccgccatgcccgaaggctacgtccaggagcgcaccatcttcttcaaggac
gacggcaactacaagacccgcgccgaggtgaagUcgagggcgacaccctggtgaaccgcatcgagctgaagggcatc
gacttcaaggaggacggcaacatcctggggcacaagctggagtacaactacaacagccacaaggtctatatcaccgccga
caagcagaagaacggcatcaaggtgaacttcaagacccgccacaacatcgaggacggcagcgtgcagctcgccgacca
ctaccagcagaacacccccatcggcgacggccccgtgctgctgcccgacaaccactacctgagcacccagtccgccctg
agcaaagaccccaacgagaagcgcgatcacatggtcctgctggagttcgtgaccgccgccgggatcactctcggcatgg
acgagctgtacaagtaacctcgactgtgccttctagttgccagccatctgUgtttgcccctcccccgtgccttccttgacccig
gaaggtgccactcccactgtcctttcctaataaaatgaggaaattgcatcgcattgtctgagtaggtgtcattctattctggggg
gtggggtggggcaggacagcaagggggaggattgggaagacaatagcaggcatgctggggatgcggtgggctctatgg
gacggatcgggagatctcccgatcccctatggtgcactctcagtacaatctgctctgatgccgcatagttaagccagtatctg
ctccctgcttgtgtgttggaggtcgctgagtagtgcgcgagcaaaatttaagctacaacaaggcaaggcttgaccgacaatt
gcatgaagaatctgcttagggttaggcgttttgcgctgcttcgcgatgtacgggccagatatacgcgttgacattgattattgac
tagttattaatagtaatcaattacggggtcattagttcatagcccatatatggagttccgcgttacataacttacggtaaatggcc
cgcctggctgaccgcccaacgacccccgcccattgacgtcaataatgacgtatgttcccatagtaacgccaatagggacttt
ccattgacgtcaatgggtggagtatttacggtaaactgcccacttggcagtacatcaagtgtatcatatgccaagtacgcccc
ctattgacgtcaatgacggtaaatggcccgcctggcattatgcccagtacatgaccttatgggactttcctacttggcagtacat
ctacgtattagtcatcgctattaccatggtgatgcggttttggcagtacatcaatgggcgtggatagcggtttgactcacgggg
atttccaagtctccaccccattgacgtcaatgggagtttgttttggaaccaaaatcaacgggactttccaaaatgtcgtaacaac
tccgccccattgacgcaaatgggcggtaggcgtgtacggtgggaggtctatataagcagagctcgtgagtttggggaccct
tgattgttctttctttttcgctattgtaaaattcatgttatatggagggggcaaagttttcagggtgttgtttagaatgggaagatgtc
ccttgtatcaccatggaccctcatgataattttgtttctttcactttctactctgttgacaaccattgtctcctcttattttcttttcattttc
tgtaactttttcgttaaactttagcttgcatttgtaacgaatttttaaattcacttttgtttatttgtcagattgtaagtactttctctaatca
cttttttttcaaggcaatcagggtatattatattgtacttcagcacagttttagagaacaattgttataattaaatgataaggtagaat
atttctgcatataaattctggctggcgtggaaatattcttattggtagaaacaactacatcctggtcatcatcctgcctttctctttat
ggttacaatgatatacactgtttgagatgaggataaaatactctgagtccaaaccgggcccctctgctaaccatgttcatgcctt
cttctttttcctacagctcctgggcaacgtgctggttattgtgctgtctcatcattttggcaaagaattgtaatacgactcactatag
ggcgaattgatatgtctagattagataaaagtaaagtgattaacagcgcattagagctgcatgtctagattagataaaagtaaa
gtgattaacagcgcattagagctgcUaatgaggtcggaatcgaaggtttaacaacccgtaaactcgcccagaagctaggtg
tagagcagcctacattgtattggcatgtaaaaaataagcgggctttgctcgacgccttagccattgagatgttagataggcacc
atactcacttttgccctttagaaggggaaagctggcaagattttttacgtaataacgctaaaagttttagatgtgctttactaagtc
atcgcgatggagcaaaagtacatttaggtacacggcctacagaaaaacagtatgaaactctcgaaaatcaattagcctttttat
gccaacaaggtttttcactagagaatgcattatatgcactcagcgctgtggggcattttactttaggttgcgtattggaagatca
agagcatcaagtcgctaaagaagaaagggaaacacctactactgatagtatgccgccattattacgacaagctatcgaattat
ttgatcaccaaggtgcagagccagccttcttattcggccttgaattgatcatatgcggattagaaaaacaacttaaatgtgaaa
gtgggtccgcgtacagcggatcccgggaattcagatcttattaaagcagaacttgtttattgcagcttataatggttacaaataa
agcaatagcatcacaaatttcacaaataaagcatttttltcactgcattctagttgtggtttgtccaaactcatcaatgtatcttatca
tgtctggtcaatgtgtgtcagttagggtgtggaaagtccccaggctccccagcaggcagaagtatgcaaagcatgcatctca
attagtcagcaaccaggtgtggaaagtccccaggctccccagcaggcagaagtatgcaaagcatgcatctcaattagtcag
caaccatagtcccgcccctaactccgcccatcccgcccctaactccgcccagttccgcccattctccgccccatggctgact
aattttttttatttatgcagaggccgaggccgcctctgcctctgagctattccagaagtagtgaggaggcttttttggaggccta
ggcttttgcaaaaagctccccatagtgactggatatgttgtgttttacagtattatgtagtctgttttttatgcaaaatctaatttaata
tattgatatttatatcattttacgtttctcgttcagctttcttgtacaaagtggttgatatccagcacagtggcggccgctcgagtct
agagggcccgcggttcgaaggtaagcctatccctaaccctctcctcggtctcgattctacgcgtaccggttagtaatgagttt
ggaattaattctgtggaatgtgtgtcagttagggtgtggaaagtccccaggctccccagcaggcagaagtatgcaaagcatg
catctcaattagtcagcaaccaggtgtggaaagtccccaggctccccagcaggcagaagtatgcaaagcatgcatctcaatt
agtcagcaaccatagtcccgcccctaactccgcccatcccgcccctaactccgcccagttccgcccattctccgccccatgg
ctgactaattttttttatttatgcagaggccgaggccgcctctgcctctgagctattccagaagtagtgaggaggcttttttggag
gcctaggcttttgcaaaaagctcccgggagcttgtatatccattttcggatctgatcagcacgtgttgacaattaatcatcggca
tagtatatcggcatagtataatacgacaaggtgaggaactaaaccatggccaagcctttgtctcaagaagaatccaccctcatt
gaaagagcaacggctacaatcaacagcatccccatctctgaagactacagcgtcgccagcgcagctctctctagcgacgg
ccgcatcttcactggtgtcaatgtatatcattttactgggggaccttgtgcagaactcgtggtgctgggcactgctgctgctgcg
gcagctggcaacctgacttgtatcgtcgcgatcggaaatgagaacaggggcatcttgagcccctgcggacggtgccgaca
ggtgcttctcgatctgcatcctgggatcaaagccatagtgaaggacagtgatggacagccgacggcagttgggattcgtga
attgctgccctctggtlatgtgtgggagggctaagcacaattcgagctcggtacctttaagaccaatgacttacaaggcagct
gtagatcttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagt
gtggaaaatctctagcagtagtagttcatgtcatcttattattcagtatttataacttgcaaagaaatgaatatcagagagtgaga
ggaacttgtttattgcagcttataatggttacaaataaagcaatagcatcacaaatttcacaaataaagcatttttttcactgcattc
tagttgtggtttgtccaaactcatcaatgtatcttatcatgtctggctctagctatcccgcccctaactccgcccatcccgcccct
aactccgcccagttccgcccattctccgccccatggctgactaattttttttatttatgcagaggccgaggccgcctcggcctct
gagctattccagaagtagtgaggaggcttttttggaggcctagggacgtacccaattcgccctatagtgagtcgtattacgcg
cgctcactggccgtcgttttacaacgtcgtgactgggaaaaccctggcgttacccaacttaatcgccttgcagcacatccccc
tttcgccagctggcgtaatagcgaagaggcccgcaccgatcgccctlcccaacagttgcgcagcctgaatggcgaatggg
acgcgccctgtagcggcgcattaagcgcggcgggtgtggtggttacgcgcagcgtgaccgctacacttgccagcgcccta
gcgcccgctcctttcgctttcttcccttcctttctcgccacgttcgccggctttccccgtcaagctctaaatcgggggctcccttt
agggttccgatttagtgctttacggcacctcgaccccaaaaaacttgattagggtgatggttcacgtagtgggccatcgccct
gatagacggtttttcgccctttgacgttggagtccacgttctttaatagtggactcttgttccaaactggaacaacactcaaccct
atctcggtctattcttttgatttataagggattttgccgatttcggcctattggttaaaaaatgagctgatttaacaaaaatttaacgc
gaattttaacaaaatattaacgcttacaatttaggtggcacttttcggggaaatgtgcgcggaacccctatttgtttatttttctaaa
tacattcaaatatgtatccgctcatgagacaataaccctgataaatgcttcaataatattgaaaaaggaagagtatgagtattca
acatttccgtgtcgcccttattcccttttttgcggcattttgccttcctgtttttgctcacccagaaacgctggtgaaagtaaaagat
gctgaagatcagttgggtgcacgagtgggttacatcgaactggatctcaacagcggtaagatccttgagagttttcgccccg
aagaacgttttccaatgatgagcacttttaaagttctgctatgtggcgcggtattatcccgtattgacgccgggcaagagcaac
tcggtcgccgcatacactattctcagaatgacttggttgagtactcaccagtcacagaaaagcatcttacggatggcatgaca
gtaagagaattatgcagtgctgccataaccatgagtgataacactgcggccaacttacttctgacaacgatcggaggaccga
aggagctaaccgcttttttgcacaacatgggggatcatgtaactcgccttgatcgttgggaaccggagctgaatgaagccata
ccaaacgacgagcgtgacaccacgatgcctgtagcaatggcaacaacgttgcgcaaactattaactggcgaactacttactc
tagcttcccggcaacaattaatagactggatggaggcggataaagttgcaggaccacttctgcgctcggcccttccggctgg
ctggtttattgctgataaatctggagccggtgagcgtgggtctcgcggtatcattgcagcactggggccagatggtaagccct
cccgtatcgtagttatctacacgacggggagtcaggcaactatggatgaacgaaatagacagatcgctgagataggtgcct
cactgattaagcattggtaactgtcagaccaagtttactcatatatactttagattgatttaaaacttcatttttaatttaaaaggatct
aggtgaagatcctttttgataatctcatgaccaaaatcccttaacgtgagttttcgttccactgagcgtcagaccccgtagaaaa
gatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgttcttctagt
gtagccgtagttaggccaccacttcaagaactctgtagcaccgcctacatacctcgctctgctaatcctgttaccagtggctgc
tgccagtggcgataagtcgtgtcttaccgggttggactcaagacgatagttaccggataaggcgcagcggtcgggctgaac
ggggggttcgtgcacacagcccagcttggagcgaacgacctacaccgaactgagatacctacagcgtgagctatgagaa
agcgccacgcttcccgaagggagaaaggcggacaggtatccggtaagcggcagggtcggaacaggagagcgcacga
gggagcttccagggggaaacgcctggtatctttatagtcctgtcgggtttcgccacctctgacttgagcgtcgatttttgtgatg
ctcgtcaggggggcggagcctatggaaaaacgccagcaacgcggcctttttacggttcctggccttttgctggccttttgctc
acatgttctttcctgcgttatcccctgattctgtggataaccgtattaccgcctttgagtgagctgataccgctcgccgcagccg
aacgaccgagcgcagcgagtcagtgagcgaggaagcggaagagcgcccaatacgcaaaccgcctctccccgcgcgtt
ggccgattcattaatgcagctggcacgacaggtttcccgactggaaagcgggcagtgagcgcaacgcaattaatgtgagtt
agctcactcattaggcaccccaggctttacactttatgcttccggctcgtatgttgtgtggaattgtgagcggataacaatttcac
acaggaaacagctatgaccatgattacgccaagcgcgcaattaaccctcactaaagggaacaaaagctggagctgcaagc
tt.

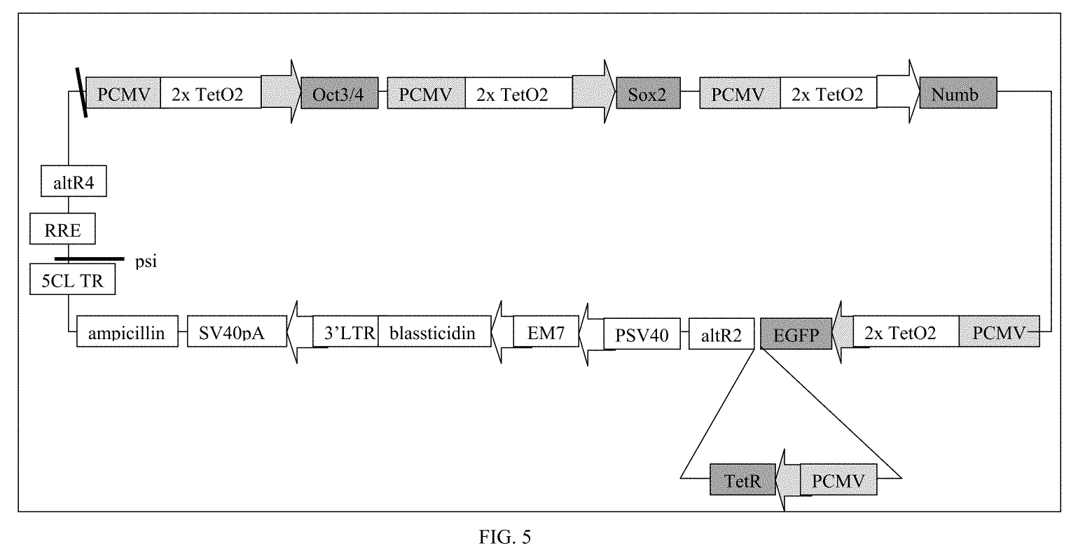

A schematized map corresponding to the vector sequence above is shown in FIG. 5.

The vector may be constructed fully through de novo gene synthesis, or in part through the cloning of the Numb, Sox and OCT3/4 cDNA sequences into the position occupied by LacZ in the Invitrogen pcDNA4tolacZ vector. Similarly, the tetR gene is found in the Invitrogen pcDNA6/TR vector. Coding sequences of genes referenced are also appropriate for cloning into the pcDNA41acZ vector.

Alternatively, the tetR gene may be transfected into target cells separately utilizing the pcDNA6/TR vector in combination with a vector comprising the sequence here minus the tetR gene and its PCMV promoter.

Likewise, multiple vectors may be employed so long as elements similar to the elements included in the above sequence are present. This may reduce the likelihood of promoter competition. It is to be understood that other conditional promoter elements may be substituted for the tetracycline sensitive promoter elements.

### Example 14

It is expected that intravenous and other administration of pluripotent stem cells produced according to the methods described herein (or other published methods) one or more times can provide replacement cells to the body and that such administration may serve to extend the life or improve the health of the patient suffering age-related senescence.

### Example 15: Production of Germ Cells

The current invention covers the derivation of germ cells from multipotent, pluripotent, and/or self-renewing stem cells produced according to the methods described herein (or according to other published methods). The production of such germ cells may be suitable for treating infertility and producing embryos in vitro (e.g. Ilubner et al., 2003; Kehler et al., 2005; Nayernia et al., 2006a; Nayernia et al., 2006b; Drusenheimer et al., 2007; Moore et al., 2007; etc.)

### Example 16: Generation of Transgenic Animals

The present invention covers the generation of transgenic animals. As with other pluripotent cells, the pluripotent cells produced by the methods described herein (or other published methods) may be utilized to produce transgenic animals by any method known to the art.

### Example 17: Therapeutic Vector Construction

Examples of retroviral vectors which may be employed include, but are not limited to, those derived from Moloney Murine Leukemia Virus, Moloney Murine Sarcoma Virus, and Rous Sarcoma Virus, FIV, and HIV. Appropriate expression vectors are that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to, lentiviral vectors, adenoviral vectors, adeno-associated viral vectors, and retroviral vectors.

The replication incompetent pcDNA 6.2 GW/miR and pcDNA 6.2/EmGFP-Bsd/V5-DEST vectors are examples of an appropriate expression vectors (Invitrogen) and allow expression of synthetic oligonucleotides (e.g. miRNAs) that have the capacity to cleave targeted sequences. These vectors include flanking and loop sequences from endogenous miRNA to direct the excision of the engineered miRNA from a longer Pol II transcript (pre-miRNA).

Alternatively, inclusion of the HIV psi sequence allows the therapeutic vector to compete with native HIV genome for packaging into viral particles, also inhibiting HIV transmission.

Combining multiple miRNA sequences directed against a single target increases the likelihood of success in reducing target sequence expression. miRNA sequences may be operably linked to tissue specific promoters such as the EF-I alpha promoter, any T cell specific promoter, or macrophage specific promoter to ensure expression in the desired cell types.

Utilizing Invitrogen's lentiviral destination (DEST) vectors for gene expression, the resulting therapeutic vector(s) becomes capable of stably transducing both dividing and non-dividing cell types.

In a preferred embodiment, the therapeutic vector(s) contains multiple synthetic oligonucleotide sequences driven by one or more promoters so as to reduce expression of CXCR4, CCR5, and/or any other cellular protein known to act as a co-receptor for HIV infection in target cells.

In one therapeutic vector (constructed in 2006), four miRNA sequences targeting CXCR4 and CCR5 co-receptors were cloned into the pcDNA 6.2 GW/miR vector along with decoy RNA sequences targeting HIV-2 TAR and RRE.

Genetic constructs may include a vector backbone, and a transactivator which regulates a promoter operably linked to heterologous nucleic acid sequences.

Another example of a suitable vector is a retroviral vector. Retroviruses are RNA viruses which contain an RNA genome. The gag, pol, and env genes are flanked by long terminal repeat (LTR) sequences. The 5′ and 3′ LTR sequences promote transcription and polyadenylation of mRNA's.

The retroviral vector may provide a regulable transactivating element, an internal ribosome reentry site (IRES), a selection marker, and a target heterologous gene operated by a regulable promoter.

Alternatively, multiple sequences may be expressed under the control of multiple promoters. Finally, the retroviral vector may contain cis-acting sequences necessary for reverse transcription and integration. Upon infection, the RNA is reverse transcribed to DNA which integrates efficiently into the host genome. The recombinant retrovirus of this invention is genetically modified in such a way that some of the retroviral, infectious genes of the native virus are removed and in embodiments replaced instead with a target nucleic acid sequence for genetic modification of the cell. The sequences may be exogenous DNA or RNA, in its natural or altered form.

### Example 18: Example Methods for Generation of the Therapeutic Vector

The methods for generation of the therapeutic vector(s) include those taught in Invitrogen's Viral Power Lentiviral Expression Systems Manual (incorporated by reference herein). Briefly, the EmGFP-bsd cassette is cloned as a PmII-BIpI fragment into the pLenti6/R4R2/V5-DEST vector, while the miR-decoy cassette is simultaneously transferred by BP reaction into pDONR221. Then the EFIa promoter and miR-decoy are Multisite LR crossed into the modified pLenti6/KmGFP-bsd/R4R2-DES Tvector.

pLenti6/R4R2/V5-DEST vector sequence:
(SEQ ID NO: 1)
aatgtagtcttatgcaatactcttgtagtcttgcaacatggtaacgatgagttagcaacatgccttacaaggagagaaaaagca
ccgtgcatgccgattggtggaagtaaggtggtacgatcgtgccttattaggaaggcaacagacgggtctgacatggattgg
acgaaccactgaattgccgcattgcagagatattgtattlaagtgcctagctcgatacataaacgggtctctctggttagacca
gatctgagcctgggagctctclggctaactagggaacccactgcttaagcctcaataaagcttgccttgagtgcttcaagtagt
gtgtgcccgtctgttgtgtgactctggtaactagagatccctcagacccttttagtcagtgtggaaaatctctagcagtggcgc
ccgaacagggacttgaaagcgaaagggaaaccagaggagctctctcgacgcaggactcggcttgctgaagcgcgcacg
gcaagaggcgaggggcggcgactggtgagtacgccaaaaattttgactagcggaggctagaaggagagagatgggtgc
gagagcgtcagtattaagcgggggagaattagatcgcgatgggaaaaaattcggttaaggccagggggaaagaaaaaat
ataaattaaaacatatagtatgggcaagcagggagclagaacgattcgcagttaatcctggcctgttagaaacatcagaagg
ctgtagacaaatactgggacagctacaaccatcccttcagacaggatcagaagaacttagatcattatataatacagtagcaa
ccctctattgtgtgcatcaaaggatagagataaaagacaccaaggaagctttagacaagatagaggaagagcaaaacaaaa
gtaagaccaccgcacagcaagcggccgctgatcttcagacctggaggaggagatatgagggacaattggagaagtgaatt
atataaatataaagtagtaaaaattgaaccattaggagtagcacccaccaaggcaaagagaagagtggtgcagagagaaaa
aagagcagtgggaataggagctttgttccttgggttcttgggagcagcaggaagcactatgggcgcagcgtcaatgacgct
gacggtacaggccagacaattattgtctggtatagtgcagcagcagaacaatttgctgagggctattgaggcgcaacagcat
ctgttgcaactcacagtctggggcatcaagcagctccaggcaagaatcctggctgtggaaagatacctaaaggatcaacag
ctcctggggatttggggttgctctggaaaactcatttgcaccactgctgtgccttggaatgctagttggagtaataaatctctgg
aacagatttggaatcacacgacctggatggagtgggacagagaaattaacaattacacaagcttaatacactccttaattgaa
gaatcgcaaaaccagcaagaaaagaatgaacaagaattattggaattagataaatgggcaagtttgtggaattggtttaacat
aacaaattggctgtggtatataaaattattcataatgatagtaggaggcttggtaggtttaagaatagtttttgctgtactttctata
gtgaatagagttaggcagggatattcaccattatcgtttcagacccacctcccaaccccgaggggacccgacaggcccgaa
ggaatagaagaagaaggtggagagagagacagagacagatccattcgattagtgaacggatctcgacggtatcgatgtcg
acgttaacgctagtgatatcaactttgtatagaaaagttgaacgagaaacgtaaaatgatataaatatcaatatattaaattagat
tttgcataaaaaacagactacataatactgtaaaacacaacatatccagtcactatggcggccgcattaggcaccccaggcttt
acactttatgcttccggctcgtataatgtgtggattttgagttaggatccgtcgagattttcaggagctaaggaagctaaaatgg
agaaaaaaatcactggatataccaccgttgatatalcccaatggcatcgtaaagaacaUttgaggcatttcagtcagttgctc
aatgtacctataaccagaccgttcagctggatattacggcctttttaaagaccgtaaagaaaaataagcacaagttttatccgg
cctttattcacattcttgcccgcctgatgaatgctcatccggaattccgtatggcaatgaaagacggtgagctggtgatatggg
atagtgttcacccttgttacaccgttttccatgagcaaactgaaacgttttcatcgctctggagtgaataccacgacgatttccgg
cagtttctacacatatattcgcaagatgtggcgtgttacggtgaaaacctggcctatttccctaaagggtttattgagaatatgttt
ttcgtctcagccaatccctgggtgagtttcaccagttttgatttaaacgtggccaatatggacaacttcttcgcccccgttttcac
catgggcaaatattatacgcaaggcgacaaggtgctgatgccgctggcgattcaggUcatcatgccgtttgtgatggcttcc
atgtcggcagaatgcttaatgaattacaacagtactgcgatgagtggcagggcggggcgtaaagatctggatccggcttact
aaaagccagataacagtatgcgtatttgcgcgctgatttttgcggtataagaatatatactgatatgtatacccgaagtatgtca
aaaagaggtatgctatgaagcagcgtattacagtgacagttgacagcgacagctatcagttgctcaaggcatatatgatgtca
atatctccggtctggtaagcacaaccatgcagaatgaagcccgtcgtctgcgtgccgaacgctggaaagcggaaaatcag
gaagggatggctgaggtcgcccggtttattgaaatgaacggctcttttgctgacgagaacagggactggtgaaatgcagtlt
aaggttlacacctataaaagagagagccgttatcgtctgtttgtggatgtacagagtgatattattgacacgcccgggcgacg
gatggtgatccccctggccagtgcacgtctgctgtcagataaagtctcccgtgaactttacccggtggtgcatatcggggatg
aaagctggcgcatgatgaccaccgatatggccagtgtgccggtctccgttatcggggaagaagtggctgatctcagccacc
gcgaaaatgacatcaaaaacgccattaacctgatgttctggggaatataaatgtcaggctccgttatacacagccagtctgca
ggtcgaccatagtgactggatatgttgtgttttacagtattatgtagtctgttttttatgcaaaatctaatttaatatattgatatttatat
cattttacgtttctcgttcagctttcttgtacaaagtggttgatatccagcacagtggcggccgctcgagtctagagggcccgc
ggttcgaaggtaagcctatccctaaccctctcctcggtctcgattctacgcgtaccggttagtaatgagtttggaattaattctgt
ggaatgtgtgtcagttagggtgtggaaagtccccaggctccccagcaggcagaagtatgcaaagcatgcatctcaattagtc
agcaaccaggtgtggaaagtccccaggctccccagcaggcagaagtatgcaaagcatgcatctcaattagtcagcaaccat
agtcccgcccctaactccgcccatcccgcccctaactccgcccagttccgcccattctccgccccatggctgactaatttttttt
attlatgcagaggccgaggccgcctctgcctctgagctattccagaagtagtgaggaggcttttttggaggcctaggcttttgc
aaaaagctcccgggagcttgtatatccattttcggatctgatcagcacgtgttgacaattaatcatcggcatagtatatcggcat
agtataatacgacaaggtgaggaactaaaccatggccaagcctttgtctcaagaagaatccaccctcattgaaagagcaacg
gctacaatcaacagcatccccatctctgaagactacagcgtcgccagcgcagctctctctagcgacggccgcatcttcactg
gtgtcaatgtatatcattttactgggggaccttgtgcagaactcgtggtgctgggcactgctgctgctgcggcagctggcaac
ctgacttgtatcgtcgcgatcggaaatgagaacaggggcatcttgagcccctgcggacggtgccgacaggtgcttctcgat
ctgcatcctgggatcaaagccatagtgaaggacagtgatggacagccgacggcagttgggattcgtgaattgctgccctct
ggttatgtgtgggagggctaagcacaattcgagctcggtacctttaagaccaatgacttacaaggcagctgtagatcttagcc
actttttaaaagaaaaggggggactggaagggctaattcactcccaacgaagacaagatctgctttttgcttgtactgggtctc
tctggttagaccagatctgagcctgggagctctctggctaactagggaacccactgcttaagcctcaataaagcttgccttga
gtgcttcaagtagtgtgtgcccgtctgttgtgtgactctggtaactagagatccctcagacccttttagtcagtgtggaaaatctc
tagcagtagtagttcatgtcatcttattattcagtatttataacttgcaaagaaatgaatatcagagagtgagaggaacttgtttatt
gcagcttataatggttacaaataaagcaatagcatcacaaatttcacaaataaagcatttttttcactgcattctagttgtggtttgt
ccaaactcatcaatgtatcttatcatgtctggctctagctatcccgcccctaactccgcccatcccgcccctaactccgcccagt
tccgcccattctccgccccatggctgactaattttttttatttatgcagaggccgaggccgcctcggcctctgagctattccaga
agtagtgaggaggcttttttggaggcctagggacgtacccaattcgccctatagtgagtcgtattacgcgcgctcactggccg
tcgttttacaacgtcgtgactgggaaaaccctggcgttacccaacttaatcgccttgcagcacatccccctttcgccagctggc
gtaatagcgaagaggcccgcaccgatcgcccttcccaacagttgcgcagcctgaatggcgaatgggacgcgccctgtag
cggcgcattaagcgcggcgggtgtggtggttacgcgcagcgtgaccgctacacttgccagcgccctagcgcccgctccttt
cgctttcttcccttcctttctcgccacgttcgccggctttccccgtcaagctctaaatcgggggctccctttagggttccgatttag
tgctttacggcacctcgaccccaaaaaacttgattagggtgatggttcacgtagtgggccatcgccctgatagacggtttttcg
ccctttgacgttggagtccacgttctttaatagtggactcttgttccaaactggaacaacactcaaccctatctcggtctattctttt
gatttataagggattttgccgatttcggcctattggttaaaaaatgagctgatttaacaaaaatttaacgcgaattttaacaaaata
ttaacgcttacaatttaggtggcacttttcggggaaatgtgcgcggaacccctatttgtttatttttctaaatacattcaaatatgtat
ccgctcatgagacaataaccctgataaatgcttcaataatattgaaaaaggaagagtatgagtattcaacatttccgtgtcgcc
cttattcccttttttgcggcattttgccttcctgtttttgctcacccagaaacgctggtgaaagtaaaagatgctgaagatcagttg
ggtgcacgagtgggttacatcgaactggatctcaacagcggtaagatccttgagagttttcgccccgaagaacgttttccaat
gatgagcacttttaaagttctgctatgtggcgcggtattatcccgtattgacgccgggcaagagcaactcggtcgccgcatac
actattctcagaatgacttggttgagtactcaccagtcacagaaaagcatcttacggatggcatgacagtaagagaattatgca
gtgctgccataaccatgagtgataacactgcggccaacttacttctgacaacgatcggaggaccgaaggagctaaccgcttt
tttgcacaacatgggggatcatgtaactcgccttgatcgttgggaaccggagctgaatgaagccataccaaacgacgagcg
tgacaccacgatgcctgtagcaatggcaacaacgttgcgcaaactattaactggcgaactacttactctagcttcccggcaac
aattaatagactggatggaggcggataaagttgcaggaccacttctgcgctcggcccttccggctggctggtttattgctgat
aaatctggagccggtgagcgtgggtctcgcggtatcattgcagcactggggccagatggtaagccctcccgtatcgtagtta
tctacacgacggggagtcaggcaactatggatgaacgaaatagacagatcgctgagataggtgcctcactgattaagcatt
ggtaactgtcagaccaagtttactcatatatactttagattgatttaaaacttcatttttaatttaaaaggatctaggtgaagatcctu
ttgataatctcatgaccaaaatcccttaacgtgagttttcgttccactgagcgtcagaccccgtagaaaagatcaaaggatcttc
ttgagatcctttttttctgcgcgtaatctgctgcttgcaaacaaaaaaaccaccgctaccagcggtggtttgtttgccggatcaa
gagctaccaactctttttccgaaggtaactggcttcagcagagcgcagataccaaatactgttcttctagtgtagccgtagttag
gccaccacttcaagaactctgtagcaccgcctacatacctcgctctgctaatcctgttaccagtggctgctgccagtggcgat
aagtcgtgtcttaccgggttggactcaagacgatagttaccggataaggcgcagcggtcgggctgaacggggggttcgtg
cacacagcccagcttggagcgaacgacctacaccgaactgagatacctacagcgtgagctatgagaaagcgccacgcttc
ccgaagggagaaaggcggacaggtatccggtaagcggcagggtcggaacaggagagcgcacgagggagcttccagg
gggaaacgcctggtatctttatagtcctgtcgggtttcgccacctctgacttgagcgtcgatttttgtgatgctcgtcagggggg
cggagcctatggaaaaacgccagcaacgcggcctttttacggttcctggccttttgctggccttttgctcacatgttctttcctgc
gttatcccctgattctgtggataaccgtattaccgcctttgagtgagctgataccgctcgccgcagccgaacgaccgagcgc
agcgagtcagtgagcgaggaagcggaagagcgcccaatacgcaaaccgcctctccccgcgcgttggccgattcattaat
gcagctggcacgacaggtttcccgactggaaagcgggcagtgagcgcaacgcaattaatgtgagttagctcactcattagg
caccccaggctttacactttatgcttccggctcgtatgttgtgtggaattgtgagcggataacaatttcacacaggaaacagcta
tgaccatgattacgccaagcgcgcaattaaccctcactaaagggaacaaaagctggagctgcaagctt.

Example miR-Decoy Cassette Sequence:

(SEQ ID NO: 3)
gtcgaccagtggatcctggaggcttgctgaaggctgtatgctgatcgggtgtaaactgagcttggttttggccactgactgac
caagctcattacacccgatcaggacacaaggcctgttactagcactcacatggaacaaatggcccagatcctggaggcttg
ctgaaggctgtatgctgataccaggcaggataaggccagttttggccactgactgactggccttactgcctggtatcaggac
acaaggcctgttactagcactcacatggaacaaatggcccagatcctggaggcttgctgaaggctgtatgctgtgaccagg
atgaccaatccatgttttggccactgactgacatggattgcatcctggtcacaggacacaaggcctgttactagcactcacatg
gaacaaatggcccagatcctggaggcttgctgaaggctgtatgctgatagcttggtccaacctgttagttttggccactgactg
actaacaggtgaccaagctatcaggacacaaggcctgttactagcactcacatggaacaaatggcccagatctccccagtg
gaaagacgcgcaggcaaaacgcaccacgtgacggagcgtgaccgcgcgccgagcgcgcgccaaggtcgggcagga
agagggcctatttcccatgattccttcatatttgcatatacgatacaaggctgttagagagataattagaattaatttgactgtaaa
cacaaagatattagtacaaaatacgtgacgtagaaagtaataatttcttgggtagtttgcagttttaaaattatgttttaaaatgga
ctatcatatgcttaccgtaacttgaaagtatttcgatttcttgggtttatatatcttgtggaaaggacggtgctcgcttcggcagca
cgtcgtgctagggttcttgggttttctcgcaacagcaggttctgcaatgggcgcggcgtccctgaccgtgtcggctcagtccc
ggactttactggccgggatagtgcagcaacagcaacagctgttggacgtggtcaagagacaacaagaactgttgcgactg
accgtctggggaacgaaaaacctccaggcaagagtcactgctatagagaagtacctacaggaccaggcgcggctaaattc
atggggatgtctagacctagagcggacttcggtccgctttttccccagtggaaagacgcgcaggcaaaacgcaccacgtga
cggagcgtgaccgcgcgccgagcgcgcgccaaggtcgggcaggaagagggcctatttcccatgattccttcatatttgcat
atacgatacaaggctgttagagagataattagaattaatttgactgtaaacacaaagatattagtacaaaatacgtgacgtaga
aagtaataatttcttgggtagtttgcagttttaaaattatgttttaaaatggactatcatatgcttaccgtaacttgaaagtatttcgat
ttcttgggtttatatatcttgtggaaaggacggtgctcgcttcggcagcacgtcggtcgctctgcggagaggctggcagattg
agccctgggaggttctctccagcactagcaggtagagcctgggtgttccctgctagactctcaccagtgcttggccggcact
gggcagacggctccacgcttgcttgcttaaagacctcttaataaagctgctctagacctagagcggacttcggtccgctttttt
acgtactcgag.

### Example 19: Methods for Propagating/Proliferating Stem/Progenitor Cells In Vivo

In order to obtain large numbers of target cells that are relatively resistant to 1) HIV infection and/or 2) HIV replication and/or 3) HIV transcription, progenitor/stem cells can be grown in Dulbecco's modified Minimal Essential Medium (DMEM) supplemented with glutamine, beta.-mercaptoethanol, 10% (by volume) horse serum, and human recombinant Leukemia Inhibitory Factor (LIF). The LIF replaces the need for maintaining progenitor/stem cells on feeder layers of cells, (which may also be employed) and is essential for maintaining progenitor/stem cells in an undifferentiated state.

### Example 20

Stem cells are collected from individuals, the cells are transfected/contacted with the therapeutic vectors, then prepared for transplantation by standard methods, with or without HLA typing and matching.

### Example 21

Umbilical cord blood samples are obtained from umbilical blood cord bank. The cells are then transfected/contacted with the therapeutic vector of beneficial sequences, then prepared for transplantation by standard methods, with or without HLA typing and matching. Example 22: Examples of Synthetic oligonucleotide sequences suitable for inclusion in the therapeutic vector.

Any synthetic oligonucleotide sequences that successfully reduce the protein expression of targeted sequences>70% is covered by the present invention.

Any synthetic oligonucleotide sequences that successfully reduce the ability of target cells to sustain HIV replication by >70% or to a lesser but therapeutic degree or HIV viral activity by >70% or to a lesser but therapeutic degree are also covered by this invention.

Examples of miRNA sequences include miRNA sequences derived by IVGN algorithm(Invitrogen). miRNA sequences targeting the CXCR4 gene include top strand: 5′-TGCTGATACCAGGCAGGATAAGGCCAGTTTTGGCCACTGACTGACTGGCCT TACTGCCT GGTAT-3′ (SEQ ID NO: 4) and bottom strand: 5′-CCTGATACCAGGCAGTAAGGCCAGTCAGTCAGTGGCCAAAACTGGCCTTA TCCTGCCTG GTATC-3′ (SEQ ID NO: 5); as well as top strand: 5′-TGCTGTGACCAGGATGACCAATCCATGTTTTGGCCACTGACTGACATGGAT TGCATCCTG GTCA-3′ (SEQ ID NO: 6) and bottom strand: 5′-CCTGTGACCAGGATGCAATCCATGTCAGTCAGTGGCCAAAACATGGATTG GTCATCCTG GTCAC-3′ (SEQ ID NO: 7).

Similarly, miRNA sequences targeting the CCR5 gene include top strand: 5′-TGCTGATCGGGTGTAAACTGAGCTTGGTTTTGGCCACTGACTGACCAAGCT CATTACACCCGAT-3′ (SEQ ID NO: 8) and bottom strand: 5′-CCTGATCGGGTGTAATGAGCTTGGTCAGTCAGTGGCCAAAACCAAGCTCA GTTTACACCCGATC-3′; (SEQ ID NO: 9) as well as top strand 5′-TGCTGATAGCTTGGTCCAACCTGTTAGTTTTGGCCACTGACTGACTAACAG GTGACCAAGCTAT-3′ (SEQ ID NO: 10) and bottom strand: 5′-CCTGATAGCTTGGTCACCTGTTAGTCAGTCAGTGGCCAAAACTAACAGGTT GGACCAAGCTATC-3′ (SEQ ID NO: 11).

### Example 23: Examples of Decoy RNA Suitable for Inclusion in the Therapeutic Vector

Any decoy sequences that successfully reduce the ability of target cells to sustain ITIV replication by >70% or to a lesser but therapeutic degree or HIV viral activity by >70% or to a lesser but therapeutic degree are covered by this invention.

An example TAR decoy sequence is

(SEQ ID NO: 12)
gtcgctctgcggagaggctggcagattgagccctgggaggttctctccag
cactagcaggtagagcctgggtgttccctgctagactctcaccagtgctt
ggccggcactgggcagacggctccacgcttgcttgcttaaagacctctta
ataaagctgc (Browning et al., 1999)

An example RRE decoy sequence is

(SEQ ID NO: 13)
tgctagggttcttgggttttctcgcaacagcaggttctgcaatgggcgcg
gcgtccctgaccgtgtcggctcagtcccggactttactggccgggatagt
gcagcaacagcaacagctgttggacgtggtcaagagacaacaagaactgt
tgcgactgaccgtctggggaacgaaaaacctccaggcaagagtcactgct
atagagaagtacctacaggaccaggcgcggctaaattcatggggatg
(Dillon et al., 1990).

### Example 24: Flanking Sequences Providing Stability for RNA Decoys

Examples of appropriate flanking sequences for RNA decoys are as follows:

(SEQ ID NO: 14)
GUGCUCGCUUCGGCAGCACGTCGAC---TAR
(SEQ ID NO: 15)
DECOY SEQ---UCUAGAGCGGACUUCGGUCCGCUUUU
(SEQ ID NO: 16)
GUGCUCGCUUCGGCAGCACGTCGAC---RRE
(SEQ ID NO: 17)
DECOY SEQ---UCUAGAGCGGACUUCGGUCCGCUUUU

Previously, it was demonstrated that decoy sequences flanked by hairpins on either side, 19 nucleotides (ntds) of the U6 RNA on the 5′ side as well as a 3′ stem immediately preceding a poly U terminator for POEIII, showed greater stability. This arrangement is expected to protect against 3 ′-5′ exonuclease attack, and to reduce the chances of the 3′ trailer interfering with the insert RNA folding. Since only the first 3/4 of the tRNA sequence is present, the 5′ end of the insert should be protected and export from the nucleus should be prevented (Good et al., 1997).

### Example 25: Introduction of Therapeutic Vector to the Host

In a preferred embodiment, blood stem/progenitor cells, and target cells are transfected/contacted with the therapeutic vector(s) (or associated therapeutic virus) in vivo by introduction of the therapeutic vector(s) into the host blood, tissues, or bone marrow, etc. The greatest benefit may be achieved by modifying a large number of endogenous target and stem/progenitor cells. This may be accomplished by using an appropriately-sized, catheter-like device, or needle to inject the therapeutic vector(s) into the venous or arterial circulation. In a preferred embodiment, the virus is pseudotyped with VSV-G envelope glycoprotein and native I II V-I env proteins.

### Example 26: Introduction of Genetically-Modified Cells into the Host

Blood cells, such as mature peripheral blood T lymphocytes, monocytes, macrophages, T cell progenitors, macrophage-monocyte progenitor cells, and/or pluripotent hematopoietic stem cells (such as those found in umbilical cord blood and occupying bone marrow spaces) as well as other stem/progenitor cells can be transfected/contacted using the therapeutic vector(s) in vitro. Appropriate concentrations of the therapeutic vector(s) may be those consistent with Browning et al., 1999. Subsequently, cells are expanded (propagated) in vitro, and are then transferred to the host via introduction of the cells to the venous or arterial circulation using a intravenous needle or catheter. Subsequently, cells transfected/contacted with the therapeutic vectors are able to “home” to the bone marrow and other tissues.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 27: Examples of Expressed or Targeted Transgenes/Proteins Utilized in the Present Invention

Any transgene sequences effective in fulfilling the present invention is suitable for use in the present invention. Suitable nucleotide sequences may be drawn from any species so long as the desired cells or behavior is achieved. Likewise the method of naming such sequences, either in lower case or upper case letters herein, does not imply a particular species. The following sequences stored in the NCBI database (listed by accession number) represent examples of sequences referenced above in the present application. They are also examples of specific transgene encoding sequences (cds) suitable for use in the present invention, but do not in any way limit the practice of the invention:

(SEQ ID NO: 18)
cardiotrophin1.U43030: atgagccggagggagggaagtctggaagacccccagactgattcctcagtctcacttct
tccccacttggaggccaagatccgtcagacacacagccttgcgcacctcctcaccaaatacgctgagcagctgctccagga
atatgtgcagctccagggagaccccttcgggctgcccagcttctcgccgccgcggctgccggtggccggcctgagcgcc
ccggctccgagccacgcggggctgccagtgcacgagcggctgcggctggacgcggcggcgctggccgcgctgccccc
gctgctggacgcagtgtgtcgccgccaggccgagctgaacccgcgcgcgccgcgcctgctgcgccgcctggaggacgc
ggcgcgccaggcccgggccctgggcgccgccgtggaggccttgctggccgcgctgggcgccgccaaccgcgggccc
cgggccgagccccccgccgccaccgcctcagccgcctccgccaccggggtcttccccgccaaggtgctggggctccgc
gtttgcggcctctaccgcgagtggctgagccgcaccgagggcgacctgggccagctgctgcccgggggctcggcctga;
NM_001330; NM_013246; BC064416; BC036787; BC049822.
(SEQ ID NO: 19)
CNTF: BC074964: atggctttcacagagcattcaccgctgacccctcaccgtcgggacctctgtagccgctctatctg
gctagcaaggaagattcgttcagacctgactgctcttacggaatcctatgtgaagcatcagggcctgaacaagaacatcaac
ctggactctgcggatgggatgccagtggcaagcactgatcagtggagtgagctgaccgaggcagagcgactccaagaga
accttcaagcttatcgtaccttccatgttttgttggccaggctcttagaagaccagcaggtgcattttaccccaaccgaaggtga
cttccatcaagctatacatacccttcttctccaagtcgctgcctttgcataccagatagaggagttaatgatactcctggaataca
agatcccccgcaatgaggctgatgggatgcctattaatgttggagatggtggtctctttgagaagaagctgtggggcctaaa
ggtgctgcaggagctttcacagtggacagtaaggtccatccatgaccttcgtttcatttcttctcatcagactgggatcccagc
acgtgggagccattatattgctaacaacaagaaaatgtag; NMJ300614; NM147164; NM_001842.
(SEQ ID NO: 20)
GP130: NM_175767: atgttgacgttgcagacttggctagtgcaagccttgtttattttcctcaccactgaatctacaggt
gaacttctagatccatgtggttatatcagtcctgaatctccagttgtacaacttcattctaatttcactgcagtttgtgtgctaaagg
aaaaatgtatggattattttcatgtaaatgctaattacattgtctggaaaacaaaccaltttactattcctaaggagcaatatactat
cataaacagaacagcatccagtgtcacctttacagatatagcttcattaaatattcagctcacttgcaacattcttacattcggac
agcttgaacagaatgtttatggaatcacaataatttcaggcttgcctccagaaaaacctaaaaatttgagttgcattgtgaacga
ggggaagaaaatgaggtgtgagtgggatggtggaagggaaacacacttggagacaaacttcactttaaaatctgaatggg
caacacacaagtttgctgattgcaaagcaaaacgtgacacccccacctcatgcactgttgattattctactgtgtattttgtcaac
attgaagtctgggtagaagcagagaatgcccttgggaaggttacatcagatcatatcaattttgatcctgtatataaagtgaag
cccaatccgccacataatttatcagtgatcaactcagaggaactgtctagtatcttaaaattgacatggaccaacccaagtatta
agagtgttataatactaaaatataacattcaatataggaccaaagatgcctcaacttggagccagattcctcctgaagacacag
catccacccgatcttcattcactgtccaagaccttaaaccttttacagaatatgtgtttaggattcgctgtatgaaggaagatggt
aagggatactggagtgactggagtgaagaagcaagtgggatcacctatgaagataacattgcctcctttlga; NM_002184;
EF442778.
(SEQ ID NO: 21)
IL6: BC015511:
atgaactccttctccacaagcgccttcggtccagttgccttctccctggggctgctcctggtgttgcctgctgccttccctgccc
cagtacccccaggagaagattccaaagatgtagccgccccacacagacagccactcacctcttcagaacgaattgacaaa
caaattcggtacatcctcgacggcatctcagccctgagaaaggagacatgtaacaagagtaacatgtgtgaaagcagcaaa
gaggcactggcagaaaacaacctgaaccttccaaagatggctgaaaaagatggatgcttccaatctggattcaatgaggag
acttgcctggtgaaaatcatcactggtcttttggagtttgaggtatacctagagtacctccagaacagatttgagagtagtgag
gaacaagccagagctgtgcagatgagtacaaaagtcctgatccagttcctgcagaaaaaggcaaagaatctagatgcaata
accacccctgacccaaccacaaatgccagcctgctgacgaagctgcaggcacagaaccagtggctgcaggacatgacaa
ctcatctcattctgcgcagctttaaggagttcctgcagtccagcctgagggctcttcggcaaatgtag; AB 107656.
(SEQ ID NO: 22)
HOXB4: NM_024015: atggctatgagttcttttttgatcaactcaaactatgtcgaccccaagttccctccatgcgagg
aatattcacagagcgaltacctacccagcgaccactcgcccgggtactacgccggcggccagaggcgagagagcagctt
ccagccggaggcgggctlcgggcggcgcgcggcgtgcaccgtgcagcgctacgcggcctgccgggaccctgggccc
ccgccgcctccgccaccacccccgccgcccccgccaccgcccggtctgtcccctcgggctcctgcgccgccacccgcc
ggggccctcctcccggagcccggccagcgctgcgaggcggtcagcagcagccccccgccgcctccctgcgcccagaa
ccccctgcaccccagcccgtcccactccgcgtgcaaagagcccgtcgtctacccctggatgcgcaaagttcacgtgagca
cggtaaaccccaattacgccggcggggagcccaagcgctctcggaccgcctacacgcgccagcaggtcttggagctgga
gaaggaatttcactacaaccgctacctgacacggcgccggagggtggagatcgcccacgcgctctgcctctccgagcgcc
agatcaagatctggttccagaaccggcgcatgaagtggaaaaaagaccacaagttgcccaacaccaagatccgctcgggt
ggtgcggcaggctcagccggagggccccctggccggcccaatggaggcccccgcgcgctctag; NM_010459.
(SEQ ID NO: 23)
IL6R: NM_000565: atgctggccgtcggctgcgcgctgctggctgccctgctggccgcgccgggagcggcgctgg
ccccaaggcgctgccctgcgcaggaggtggcgagaggcgtgctgaccagtctgccaggagacagcgtgactctgacct
gcccgggggtagagccggaagacaatgccactgttcactgggtgctcaggaagccggctgcaggctcccaccccagca
gatgggctggcatgggaaggaggctgctgctgaggtcggtgcagctccacgactctggaaactattcatgctaccgggcc
ggccgcccagctgggactgtgcacttgctggtggatgttccccccgaggagccccagctctcctgcttccggaagagccc
cctcagcaatgttgtttgtgagtggggtcctcggagcaccccatccctgacgacaaaggctgtgctcttggtgaggaagtltc
agaacagtccggccgaagacttccaggagccgtgccagtattcccaggagtcccagaagtlctcctgccagttagcagtcc
cggagggagacagctctttctacatagtgtccatgtgcgtcgccagtagtgtcgggagcaagttcagcaaaactcaaaccttt
cagggttgtggaatcttgcagcctgatccgcctgccaacatcacagtcactgccgtggccagaaacccccgctggctcagt
gtcacctggcaagacccccactcctggaactcatctttctacagactacggtttgagctcagatatcgggctgaacggtcaaa
gacattcacaacatggatggtcaaggacctccagcatcactgtgtcatccacgacgcctggagcggcctgaggcacgtggl
gcagcttcgtgcccaggaggagttcgggcaaggcgagtggagcgagtggagcccggaggccatgggcacgccttgga
cagaatccaggagtcctccagctgagaacgaggtgtccacccccatgcaggcacttactactaataaagacgatgataatat
tctcttcagagattctgcaaatgcgacaagcctcccagtgcaagattcttcttcagtaccactgcccacattcctggttgctgga
gggagcctggccttcggaacgctcctctgcattgccattgttctgaggttcaagaagacgtggaagctgcgggctctgaagg
aaggcaagacaagcatgcatccgccgtactctttggggcagctggtcccggagaggcctcgacccaccccagtgcttgttc
ctctcatctccccaccggtgtcccccagcagcctggggtctgacaatacctcgagccacaaccgaccagatgccagggac
ccacggagcccttatgacatcagcaatacagactacttcttccccagatag; NM_181359.
(SEQ ID NO: 24)
IL11: NM_133519:
atgaactgtgtttgtcgcctggtcctggtggtgctgagcctctggccagatagagtcgttgcccctgggccaccagctggctc
ccctcgagtgtcttcagaccctcgtgcagatctggatagcgctgtcctcttgaccaggtccctcctggcagacacacggcaa
ctagctgcacagatgagagacaaattcccagctgatggagaccacaatctggactccctacctaccttggccatgagcgctg
ggacactgggatctttgcagcttcctggagtgctgacaaggcttcgagtagacttaatgtcctacttccgacatgtacagtggt
tgcgccgggcagctggtccttccctaaagactctggagccagagctgggtgccctgcaagcccgactggaacggctactt
cgtcgcttacagctcttgatgtctcgcctagccttgccccaggcagccccggaccaacctgcggtccctctgggccctcctg
cctcggcctggggaagcatccgggcagctcatgccatcctaggagggctgcacctgaccttggactgggccgtgcgggg
cctgctgttgttaaagactcggctgtaa; NM_008350.
(SEQ ID NO: 25)
LIF: NM_002309: atgaaggtcttggcggcaggagttgtgcccctgctgttggttctgcactggaaacatggggcggg
gagccccctccccatcacccctgtcaacgccacctgtgccatacgccacccatgtcacaacaacctcatgaaccagatcag
gagccaactggcacagctcaatggcagtgccaatgccctctttattctctattacacagcccagggggagccgttccccaac
aacctggacaagctatgtggccccaacgtgacggacttcccgcccttccacgccaacggcacggagaaggccaagctgg
tggagctgtaccgcatagtcgtgtaccttggcacctccctgggcaacatcacccgggaccagaagatcctcaaccccagtg
ccctcagcctccacagcaagctcaacgccaccgccgacatcctgcgaggcctccttagcaacgtgctgtgccgcctgtgca
gcaagtaccacgtgggccatgtggacgtgacctacggccctgacacctcgggtaaggatgtcttccagaagaagaagctg
ggctgtcaactcctggggaagtataagcagatcatcgccgtgttggcccaggccttctag; NM_008501; BB235045.
(SEQ ID NO: 26)
LIFR: NM_ 002310: atgatggatatttacgtatgtttgaaacgaccatcctggatggtggacaataaaagaatgaggac
tgcttcaaatttccagtggctgttatcaacatttattcttctatatctaatgaatcaagtaaatagccagaaaaagggggctcctca
tgatttgaagtgtgtaactaacaatttgcaagtgtggaactgttcttggaaagcaccctctggaacaggccgtggtactgattat
gaagtttgcattgaaaacaggtcccgttcttgttatcagttggagaaaaccagtattaaaattccagctctttcacatggtgattat
gaaataacaataaattctctacatgattttggaagttctacaagtaaattcacactaaatgaacaaaacgtttccttaattccagat
actccagagatcttgaatttgtctgctgatttctcaacctctacattatacctaaagtggaacgacaggggttcagtttttccacac
cgctcaaatgttatctgggaaattaaagttctacgtaaagagagtatggagctcgtaaaattagtgacccacaacacaactctg
aatggcaaagatacacttcatcactggagttgggcctcagatatgcccttggaatgtgccattcattttgtggaaattagatgct
acattgacaatcttcatttttctggtctcgaagagtggagtgactggagccctgtgaagaacatttcttggatacctgattctcag
actaaggtttttcctcaagataaagtgatacttgtaggctcagacataacattttgttgtgtgagtcaagaaaaagtgttatcagc
actgattggccatacaaactgccccttgatccatcttgatggggaaaatgttgcaatcaagattcgtaatatttctgtttctgcaa
gtagtggaacaaatgtagtttttacaaccgaagataacatatttggaaccgttatttttgctggatatccaccagatactcctcaa
caactgaattgtgagacacatgatttaaaagaaattatatgtagttggaatccaggaagggtgacagcgttggtgggcccac
gtgctacaagctacactttagttgaaagtttttcaggaaaatatgttagacttaaaagagctgaagcacctacaaacgaaagct
atcaattattatttcaaatgcttccaaatcaagaaatatataattttactttgaatgctcacaatccgctgggtcgatcacaatcaac
aattttagttaatataactgaaaaagtttatccccatactcctacttcattcaaagtgaaggatattaattcaacagctgttaaacttt
cttggcatttaccaggcaactttgcaaagattaatlttttatgtgaaattgaaattaagaaatctaattcagtacaagagcagcgg
aatgtcacaatcaaaggagtagaaaattcaagttatcttgttgctctggacaagttaaatccatacactctatatacttttcggatt
cgttgttctactgaaactttctggaaatggagcaaatggagcaataaaaaacaacatttaacaacagaagccagtccttcaaa
ggggcctgatacttggagagagtggagttctgatggaaaaaatttaataatctattggaagcctttacccattaatgaagctaat
ggaaaaatactttcctacaatgtatcgtgttcatcagatgaggaaacacagtccctttctgaaatccctgatcctcagcacaaag
cagagatacgacttgataagaatgactacatcatcagcgtagtggctaaaaattctgtgggctcatcaccaccttccaaaatag
cgagtatggaaattccaaatgatgatctcaaaatagaacaagttgttgggatgggaaaggggattctcctcacctggcattac
gaccccaacatgacttgcgactacgtcattaagtggtgtaactcgtctcggtcggaaccatgccttatggactggagaaaagt
tccctcaaacagcactgaaactgtaatagaatctgatgagtttcgaccaggtataagatataattttttcctgtatggatgcagaa
atcaaggatatcaattattacgctccatgattggatatatagaagaattggctcccattgttgcaccaaattttactgttgaggata
cttctgcagattcgatattagtaaaatgggaagacattcctgtggaagaacttagaggctttttaagaggatatttgttttactttg
gaaaaggagaaagagacacatctaagatgagggttttagaatcaggtcgttctgacataaaagttaagaatattactgacata
tcccagaagacactgagaattgctgatcttcaaggtaaaacaagttaccacctggtcttgcgagcctatacagatggtggagt
gggcccggagaagagtatgtatgtggtgacaaaggaaaattctgtgggattaattattgccattctcatcccagtggcagtgg
ctgtcattgttggagtggtgacaagtatccttlgctatcggaaacgagaatggattaaagaaaccttctaccctgatattccaaa
tccagaaaactgtaaagcattacagtttcaaaagagtgtctgtgagggaagcagtgctcttaaaacattggaaatgaatccttg
taccccaaataatgttgaggttctggaaactcgatcagcatttcctaaaatagaagatacagaaataatttccccagtagctga
gcgtcctgaagatcgctctgatgcagagcctgaaaaccatgtggttgtgtcctattgtccacccatcattgaggaagaaatac
caaacccagccgcagatgaagctggagggactgcacaggttatttacattgatgttcagtcgatgtatcagcctcaagcaaa
accagaagaagaacaagaaaatgaccctgtaggaggggcaggctataagccacagatgcacctccccattaattctactgt
ggaagatatagctgcagaagaggacttagataaaactgcgggttacagacctcaggccaatgtaaatacatggaatttagtg
tctccagactctcctagatccatagacagcaacagtgagattgtctcatttggaagtccatgctccattaatlcccgacaattttt
gattcctcctaaagatgaagactctcctaaatctaatggaggagggtggtcctttacaaacttttttcagaacaaaccaaacgat
taa; NM 013584; NM_031048.
(SEQ ID NO: 27)
STAT3: NM_003 150:
atggcccaatggaatcagctacagcagcttgacacacggtacctggagcagctccatcagctctacagtgacagcttccca
atggagctgcggcagtttctggccccttggattgagagtcaagattgggcatatgcggccagcaaagaatcacatgccactt
tggtgtttcataatctcctgggagagattgaccagcagtatagccgcttcctgcaagagtcgaatgttctctatcagcacaatct
acgaagaatcaagcagtttcttcagagcaggtatcttgagaagccaatggagattgcccggattgtggcccggtgcctgtgg
gaagaatcacgccttctacagactgcagccactgcggcccagcaagggggccaggccaaccaccccacagcagccgtg
gtgacggagaagcagcagatgctggagcagcaccttcaggatgtccggaagagagtgcaggatctagaacagaaaatga
aagtggtagagaatctccaggatgactttgatttcaactataaaaccctcaagagtcaaggagacatgcaagatctgaatgga
aacaaccagtcagtgaccaggcagaagatgcagcagctggaacagatgctcactgcgctggaccagatgcggagaagc
atcgtgagtgagctggcggggcttttgtcagcgatggagtacgtgcagaaaactctcacggacgaggagctggctgactg
gaagaggcggcaacagattgcctgcattggaggcccgcccaacatctgcctagatcggctagaaaactggataacgtcatt
agcagaatctcaacttcagacccgtcaacaaattaagaaactggaggagttgcagcaaaaagtttcctacaaaggggaccc
cattgtacagcaccggccgatgctggaggagagaatcgtggagctgtttagaaacttaatgaaaagtgcctttgtggtggag
cggcagccctgcatgcccatgcatcctgaccggcccctcgtcatcaagaccggcgtccagttcactactaaagtcaggttgc
tggtcaaattccctgagttgaattatcagcttaaaattaaagtgtgcattgacaaagactctggggacgttgcagctctcagag
gatcccggaaatttaacattctgggcacaaacacaaaagtgatgaacatggaagaatccaacaacggcagcctctctgcag
aattcaaacacttgaccctgagggagcagagatgtgggaatgggggccgagccaattgtgatgcttccctgattgtgactga
ggagctgcacctgatcacctttgagaccgaggtgtatcaccaaggcctcaagattgacctagagacccactccttgccagtt
gtggtgatctccaacatctgtcagatgccaaatgcctgggcgtccatcctgtggtacaacatgctgaccaacaatcccaaga
atgtaaacttttttaccaagcccccaattggaacctgggatcaagtggccgaggtcctgagctggcagttctcctccaccacc
aagcgaggactgagcatcgagcagctgactacactggcagagaaactcttgggacctggtgtgaattattcagggtgtcag
atcacatgggctaaattttgcaaagaaaacatggctggcaagggcttctccttctgggtctggctggacaatatcattgacctt
gtgaaaaagtacatcctggccctttggaacgaagggtacatcatgggctttatcagtaaggagcgggagcgggccatcttg
agcactaagcctccaggcacctlcctgctaagattcagtgaaagcagcaaagaaggaggcgtcactttcacttgggtggag
aaggacatcagcggtaagacccagatccagtccgtggaaccatacacaaagcagcagctgaacaacatgtcatttgctgaa
atcatcatgggctataagatcatggatgctaccaatatcctggtgtctccactggtctatctctatcctgacattcccaaggagg
aggcattcggaaagtattgtcggccagagagccaggagcatcctgaagctgacccaggcgctgccccatacctgaagacc
aagtttatctgtgtgacaccaacgacctgcagcaataccattgacctgccgatgtccccccgcactttagattcattgatgcagt
ttggaaataatggtgaaggtgctgaaccctcagcaggagggcagtttgagtccctcacctttgacatggagttgacctcgga
gtgcgctacctcccccatgtga; NMJ 13662; NM_139276.
(SEQ ID NO: 28)
NUMB: AF171938:
atgaacaaattacggcaaagttttaggagaaagaaggatgtttatgttccagaggccagtcgtccacatcagtggcagacag
atgaagaaggcgttcgcaccggaaaatgtagcttcccggttaagtaccttggccatgtagaagttgatgaatcaagaggaat
gcacatctgtgaagatgctgtaaaaagattgaaagctgaaaggaagttcttcaaaggcttctttggaaaaactggaaagaaa
gcagttaaagcagttctgtgggtctcagcagatggactcagagttgtggatgaaaaaactaaggacctcatagttgaccaga
cgatagagaaagtttctttctgtgccccagacaggaactttgatagagccttttcttacatatgccgtgatggcaccactcgtcg
ctggatctgtcactgcttcatggctgtcaaggacacaggtgaaaggttgagccatgcagtaggctgtgcttttgcagcctgttt
agagcgcaagcagaagcgggagaaggaatgtggagtgactgctacttttgatgctagtcggaccacttttacaagagaagg
atcattccgtgtcacaacagccactgaacaagcagaaagagaggagatcatgaaacaaatgcaagatgccaagaaagctg
aaacagataagatagtcgttggttcatcagttgcccctggcaacactgccccatccccatcctctcccacctctcctacttctga
tgccacgacctctctggagatgaacaatcctcatgccatcccacgccggcatgctccaattgaacagcttgctcgccaaggc
tctttccgaggttttcctgctcttagccagaagatgtcaccctttaaacgccaactatccctacgcatcaatgagttgccttccac
tatgcagaggaagactgatttccccattaaaaatgcagtgccagaagtagaaggggaggcagagagcatcagctccctgtg
ctcacagatcaccaatgccttcagcacacctgaggaccccttctcatctgctccgatgaccaaaccagtgacagtggtggca
ccacaatctcctaccttccaagctaatggcactgactcagccttccatgtgcttgctaagccagcccatactgctctagcaccc
gtagcaatgcctgtgcgtgaaaccaacccttgggcccatgcccctgatgctgctaacaaggaaattgcagccacatgttcgg
ggaccgagtggggtcaatcttctggtgctgcctctccaggtctcttccaggccggtcatagacgtactccctctgaggccga
ccgatggttagaagaggtgtctaagagcgtccgggctcagcagccccaggcctcagctgctcctctgcagccagttctcca
gcctcctccacccactgccatctcccagccagcatcacctttccaagggaatgcattcctcacctctcagcctgtgccagtgg
gtgtggtcccagccctgcaaccagcctttgtccctgcccagtcctatcctgtggccaatggaatgccctatccagcccctaat
gtgcctgtggtgggcatcactccctcccagatggtggccaacgtatttggcactgcaggccaccctcaggctgcccatcccc
atcagtcacccagcctggtcaggcagcagacattccctcactacgaggcaagcagtgctaccaccagtcccttctttaagcc
tcctgctcagcacctcaacggttctgcagctttcaatggtgtagatgatggcaggttggcctcagcagacaggcatacagag
gttcctacaggcacctgcccagtggatccttttgaagcccagtgggctgcattagaaaataagtccaagcagcgtactaatcc
ctcccctaccaaccctttctccagtgacttacagaagacgtttgaaattgaactttaa;
(SEQ ID NO: 29)
AF171939: atgaacaaattacggcaaagttttaggagaaagaaggatgtttatgttccagaggccagtcgtccacatcag
tggcagacagatgaagaaggcgttcgcaccggaaaatgtagcttcccggttaagtaccttggccatgtagaagttgatgaat
caagaggaatgcacatctgtgaagatgctgtaaaaagattgaaagctgaaaggaagttcttcaaaggcttctttggaaaaact
ggaaagaaagcagttaaagcagttctgtgggtctcagcagatggactcagagttgtggatgaaaaaactaaggacctcata
gttgaccagacgatagagaaagtttctttctgtgccccagacaggaactttgatagagccttttcttacatatgccgtgatggca
ccactcgtcgctggatctgtcactgcttcatggctgtcaaggacacaggtgaaaggttgagccatgcagtaggctgtgctttt
gcagcctgtttagagcgcaagcagaagcgggagaaggaatgtggagtgactgctacttttgatgctagtcggaccactttta
caagagaaggatcattccgtgtcacaacagccactgaacaagcagaaagagaggagatcatgaaacaaatgcaagatgc
caagaaagctgaaacagataagatagtcgttggttcatcagttgcccctggcaacactgccccatccccatcctctcccacct
ctcctacttctgatgccacgacctctctggagatgaacaatcctcatgccatcccacgccggcatgctccaattgaacagcttg
ctcgccaaggctctttccgaggttttcctgctcttagccagaagatgtcaccctttaaacgccaactatccctacgcatcaatga
gttgccttccactatgcagaggaagactgatttccccattaaaaatgcagtgccagaagtagaaggggaggcagagagcat
cagctccctgtgctcacagatcaccaatgccttcagcacacctgaggaccccttctcatctgctccgatgaccaaaccagtga
cagtggtggcaccacaatctcctaccttccaagggaccgagtggggtcaatcttctggtgctgcctctccaggtctcttccag
gccggtcatagacgtactccctctgaggccgaccgatggttagaagaggtgtctaagagcgtccgggctcagcagcccca
ggcctcagctgctcctctgcagccagttctccagcctcctccacccactgccatctcccagccagcatcacctttccaaggga
atgcattcctcacctctcagcctgtgccagtgggtgtggtcccagccctgcaaccagcctttgtccctgcccagtcctatcctg
tggccaatggaatgccctatccagcccctaatgtgcctgtggtgggcatcactccctcccagatggtggccaacgtatttggc
actgcaggccaccctcaggctgcccatccccatcagtcacccagcctggtcaggcagcagacattccctcactacgaggc
aagcagtgctaccaccagtcccttctttaagcctcctgctcagcacctcaacggttctgcagctttcaatggtgtagatgatgg
caggttggcctcagcagacaggcatacagaggttcctacaggcacctgcccagtggatccttttgaagcccagtgggctgc
attagaaaataagtccaagcagcgtactaatccctcccctaccaaccctttctccagtgacttacagaagacgtttgaaattga
actttaa;
(SEQ ID NO: 30)
AF171940:
atgaacaaattacggcaaagttttaggagaaagaaggatgtttatgttccagaggccagtcgtccacatcagtggcagacag
atgaagaaggcgttcgcaccggaaaatgtagcttcccggttaagtaccttggccatgtagaagttgatgaatcaagaggaat
gcacatctgtgaagatgctgtaaaaagattgaaagctactggaaagaaagcagttaaagcagttctgtgggtctcagcagat
ggactcagagttgtggatgaaaaaactaaggacctcatagttgaccagacgatagagaaagtttctttctgtgccccagaca
ggaactttgatagagccttttcttacatatgccgtgatggcaccactcgtcgctggatctgtcactgcttcatggctgtcaagga
cacaggtgaaaggttgagccatgcagtaggctgtgcttttgcagcctgtttagagcgcaagcagaagcgggagaaggaat
gtggagtgactgctacttttgatgctagtcggaccacttttacaagagaaggatcattccgtgtcacaacagccactgaacaa
gcagaaagagaggagatcatgaaacaaatgcaagatgccaagaaagctgaaacagataagatagtcgttggttcatcagtt
gcccctggcaacactgccccatccccatcctctcccacctctcctacttctgatgccacgacctctctggagatgaacaatcct
catgccatcccacgccggcatgctccaattgaacagcttgctcgccaaggctctttccgaggttttcctgctcttagccagaag
atgtcaccctttaaacgccaactatccctacgcatcaatgagttgccttccactatgcagaggaagactgatttccccattaaaa
atgcagtgccagaagtagaaggggaggcagagagcatcagctccctgtgctcacagatcaccaatgccttcagcacacct
gaggaccccttctcatctgctccgatgaccaaaccagtgacagtggtggcaccacaatctcctaccttccaagctaatggca
ctgactcagccttccatgtgcttgctaagccagcccatactgctctagcacccgtagcaatgcctgtgcgtgaaaccaaccctt
gggcccatgcccctgatgctgctaacaaggaaattgcagccacatgttcggggaccgagtggggtcaatcttctggtgctg
cctctccaggtctcttccaggccggtcatagacgtactccctctgaggccgaccgatggttagaagaggtgtctaagagcgt
ccgggctcagcagccccaggcctcagctgctcctctgcagccagttctccagcctcctccacccactgccatctcccagcc
agcatcacctttccaagggaatgcattcctcacctctcagcctgtgccagtgggtgtggtcccagccctgcaaccagcctttg
tccctgcccagtcctatcctgtggccaatggaatgccctatccagcccctaatgtgcctgtggtgggcatcactccctcccag
atggtggccaacgtatttggcactgcaggccaccctcaggctgcccatccccatcagtcacccagcctggtcaggcagcag
acattccctcactacgaggcaagcagtgctaccaccagtcccttctltaagcctcctgctcagcacctcaacggttctgcagct
ttcaatggtgtagatgatggcaggttggcctcagcagacaggcatacagaggtlcctacaggcacctgcccagtggatcctt
ttgaagcccagtgggctgcattagaaaataagtccaagcagcgtactaatccctcccctaccaaccctttctccagtgacttac
agaagacgtttgaaattgaactttaa;
(SEQ ID NO: 31)
AF171941:
atgaacaaattacggcaaagttttaggagaaagaaggatgtttatgttccagaggccagtcgtccacatcagtggcagacag
atgaagaaggcgttcgcaccggaaaatgtagcttcccggttaagtaccttggccatgtagaagttgatgaatcaagaggaat
gcacatctgtgaagatgctgtaaaaagattgaaagctactggaaagaaagcagttaaagcagttctgtgggtctcagcagat
ggactcagagttgtggatgaaaaaactaaggacctcatagttgaccagacgatagagaaagtttctttctgtgccccagaca
ggaactttgatagagccttttcttacatatgccgtgatggcaccactcgtcgctggatctgtcactgcttcatggctgtcaagga
cacaggtgaaaggttgagccatgcagtaggctgtgcttttgcagcctgtttagagcgcaagcagaagcgggagaaggaat
gtggagtgactgctacttttgatgctagtcggaccacttttacaagagaaggatcattccgtgtcacaacagccactgaacaa
gcagaaagagaggagatcatgaaacaaatgcaagatgccaagaaagctgaaacagataagatagtcgttggttcatcagtt
gcccctggcaacactgccccatccccatcctctcccacctctcctacttctgatgccacgacctctctggagatgaacaatcct
catgccatcccacgccggcatgctccaattgaacagcttgctcgccaaggctctttccgaggttttcctgctcttagccagaag
atgtcaccctttaaacgccaactatccctacgcatcaatgagttgccttccactatgcagaggaagactgatttccccattaaaa
atgcagtgccagaagtagaaggggaggcagagagcatcagctccctgtgctcacagatcaccaatgccttcagcacacct
gaggaccccttctcatctgctccgatgaccaaaccagtgacagtggtggcaccacaatctcctaccttccaagggaccgagt
ggggtcaatcttctggtgctgcctctccaggtctcttccaggccggtcatagacgtactccctctgaggccgaccgatggtta
gaagaggtgtctaagagcgtccgggctcagcagccccaggcctcagctgctcctctgcagccagttctccagcctcctcca
cccactgccatctcccagccagcatcacctttccaagggaatgcattcctcacctctcagcctgtgccagtgggtgtggtccc
agccctgcaaccagcctttgtccctgcccagtcctatcctgtggccaatggaatgccctatccagcccctaatgtgcctgtggt
gggcatcactccctcccagatggtggccaacgtatttggcactgcaggccaccctcaggctgcccatccccatcagtcacc
cagcctggtcaggcagcagacattccctcactacgaggcaagcagtgctaccaccagtcccttctttaagcctcctgctcag
cacctcaacggttctgcagctttcaatggtgtagatgatggcaggttggcctcagcagacaggcatacagaggttcctacag
gcacctgcccagtggatccttttgaagcccagtgggctgcattagaaaataagtccaagcagcgtactaatccctcccctacc
aaccctttctccagtgacttacagaagacgtttgaaattgaactttaa; NMJ 10949; NMJ 33287;
BB483123; NM 010950; NM_010949; NM 004756; DQ022744.
(SEQ ID NO: 32)
Numblike: NM 00475:
atgtcccgcagcgcggcggccagcggcggaccccggaggcctgagcggcacctgcccccagccccctgtggggcccc
ggggcccccagaaacctgcaggacggagccagacggggcgggcaccatgaacaagttacggcagagcctgcggcgg
aggaagccagcctacgtgcccgaggcgtcgcgcccgcaccagtggcaggcagacgaggacgcggtgcggaagggca
cgtgcagcttcccggtcaggtacctgggtcacgtggaggtagaggagtcccggggaatgcacgtgtgtgaagatgcggtg
aagaagctgaaggcgatgggccgaaagtccgtgaagtctgtcctgtgggtgtcagccgatgggctccgagtggtggacga
caaaaccaaggatcttctggtcgaccagaccatcgaaaaggtctccttttgtgctcctgaccgcaacctggacaaggctttct
cctatatctgtcgtgacgggactacccgccgctggatctgccactgttttctggcactgaaggactccggcgagaggctgag
ccacgctgtgggctgtgcttttgccgcctgcctggagcgaaaacagcgacgggagaaggaatgtggggtcacggccgcc
ttcgatgccagccgcaccagcttcgcccgcgagggctccttccgcctgtctgggggtgggcggcctgctgagcgagagg
ccccggacaagaagaaagcagaggcagcagctgcccccactgtggctcctggccctgcccagcctgggcacgtgtcccc
gacaccagccaccacatcccctggtgagaagggtgaggcaggcacccctgtggctgcaggcaccactgcggccgccat
cccccggcgccatgcacccctggagcagctggttcgccagggctccttccgtgggttcccagcactcagccagaagaact
cgcctttcaaacggcagctgagcctacggctgaatgagctgccatccacgctgcagcgccgcactgacttccaggtgaag
ggcacagtgcctgagatggagcctcctggtgccggcgacagtgacagcatcaacgctctgtgcacacagatcagttcatct
tttgccagtgctggagcgccagcaccagggccaccacctgccacaacagggacttctgcctggggtgagccctccgtgcc
ccctgcagctgccttccagcctgggcacaagcggacaccttcagaggctgagcgatggctggaggaggtgtcacaggtg
gccaaggcccagcagcagcagcagcagcaacagcaacagcagcagcagcagcagcagcaacagcagcaagcagcct
cagtggccccagtgcccaccatgcctcctgccctgcagcctltccccgcccccgtggggccctttgacgctgcacctgccc
aagtggccgtgttcctgccacccccacacatgcagcccccttttgtgcccgcctacccgggcttgggctacccaccgatgcc
ccgggtgcccgtggtgggcatcacaccctcacagatggtggcaaacgccttctgctcagccgcccagctccagcctcagc
ctgccactctgcttgggaaagctggggccttcccgccccctgccatacccagtgcccctgggagccaggcccgccctcgc
cccaatggggccccctggccccctgagccagcgcctgccccagctccagagttggacccctttgaggcccagtgggcgg
cattagaaggcaaagccactgtagagaaaccctccaaccccttttctggcgacctgcaaaagacattcgagattgaactgta
g; U96441; NM O 10950; DQ022744.
(SEQ ID NO: 33)
NANOG: NM 024865:
atgagtgtggatccagcttgtccccaaagcttgccttgctttgaagcatccgactgtaaagaatcttcacctatgcctgtgatttg
tgggcctgaagaaaactatccatccttgcaaatgtcttctgctgagatgcctcacacggagactgtctctcctcttccttcctcc
atggatctgcttattcaggacagccctgattcttccaccagtcccaaaggcaaacaacccacttctgcagagaagagtgtcgc
aaaaaaggaagacaaggtcccggtcaagaaacagaagaccagaactgtgttctcttccacccagctgtgtgtactcaatgat
agatttcagagacagaaatacctcagcctccagcagatgcaagaactctccaacatcctgaacctcagctacaaacaggtga
agacctggttccagaaccagagaatgaaatctaagaggtggcagaaaaacaactggccgaagaatagcaatggtgtgacg
cagaaggcctcagcacctacctaccccagcctttactcttcctaccaccagggatgcctggtgaacccgactgggaaccttc
caatgtggagcaaccagacctggaacaattcaacctggagcaaccagacccagaacatccagtcctggagcaaccactcc
tggaacactcagacctggtgcacccaatcctggaacaatcaggcctggaacagtcccttctataactgtggagaggaatctc
tgcagtcctgcatgcagttccagccaaattctcctgccagtgacttggaggctgccttggaagctgctggggaaggccttaat
gtaatacagcagaccactaggtattttagtactccacaaaccatggatttattcctaaactactccatgaacatgcaacctgaag
acgtgtga; BC 137873; NM 028016; NM_013633; BC160187.
(SEQ ID NO: 34)
OncostatinM(OSM): NM_020530: atgggggtactgctcacacagaggacgctgctcagtctggtccttgcact
cctgtttccaagcatggcgagcatggcggctataggcagctgctcgaaagagtaccgcgtgctccttggccagctccagaa
gcagacagatctcatgcaggacaccagcagactcctggacccctatatacgtatccaaggcctggatgttcctaaactgaga
gagcactgcagggagcgccccggggccttccccagtgaggagaccctgagggggctgggcaggcggggcttcctgca
gaccctcaatgccacactgggctgcgtcctgcacagactggccgacttagagcagcgcctccccaaggcccaggatttgg
agaggtctgggctgaacatcgaggacttggagaagctgcagatggcgaggccgaacatcctcgggctcaggaacaacat
ctactgcatggcccagctgctggacaactcagacacggctgagcccacgaaggctggccggggggcctctcagccgccc
acccccacccctgcctcggatgcttttcagcgcaagctggagggctgcaggttcctgcatggctaccatcgcttcatgcactc
agtggggcgggtcttcagcaagtggggggagagcccgaaccggagccggagacacagcccccaccaggccctgagg
aagggggtgcgcaggaccagaccctccaggaaaggcaagagactcatgaccaggggacagctgccccggtag;
NM_001013365; NP_065391; NP_001013383.
(SEQ ID NO: 35)
OSMR: NM_003999: atggctctatttgcagtctttcagacaacattcttcttaacattgctgtccttgaggacttaccag
agtgaagtcttggctgaacgtttaccattgactcctgtatcacttaaagtttccaccaattctacgcgtcagagtttgcacttacaa
tggactgtccacaaccttccttatcatcaggaattgaaaatggtatttcagatccagatcagtaggattgaaacatccaatgtca
tctgggtggggaattacagcaccactgtgaagtggaaccaggttctgcattggagctgggaatctgagctccctttggaatgt
gccacacactttgtaagaataaagagtttggtggacgatgccaagttccctgagccaaatttctggagcaactggagttcctg
ggaggaagtcagtgtacaagattctactggacaggatatattgttcgttttccctaaagataagctggtggaagaaggcacca
atgttaccatttgttacgtttctaggaacattcaaaataatgtatcctgttatttggaagggaaacagattcatggagaacaacttg
atccacatgtaactgcattcaacttgaatagtgtgcctttcattaggaataaagggacaaatatctattgtgaggcaagtcaagg
aaatgtcagtgaaggcatgaaaggcatcgttctttttgtctcaaaagtacttgaggagcccaaggacttttcttgtgaaaccga
ggacttcaagactttgcactgtacttgggatcctgggacggacactgccttggggtggtctaaacaaccttcccaaagctaca
ctttatttgaatcattttctggggaaaagaaactttgtacacacaaaaactggtgtaattggcaaataactcaagactcacaaga
aacctataacttcacactcatagctgaaaattacttaaggaagagaagtgtcaatatcctttttaacctgactcatcgagtttattt
aatgaatccttttagtgtcaactttgaaaatgtaaatgccacaaatgccatcatgacctggaaggtgcactccataaggaataat
ttcacatatttgtgtcagattgaactccatggtgaaggaaaaatgatgcaatacaatgtttccatcaaggtgaacggtgagtact
tcttaagtgaactggaacctgccacagagtacatggcgcgagtacggtgtgctgatgccagccacttctggaaatggagtga
atggagtggtcagaacttcaccacacttgaagctgctccctcagaggcccctgatgtctggagaattgtgagcttggagcca
ggaaatcatactgtgaccttattctggaagccattatcaaaactgcatgccaatggaaagatcctgttctataatgtagttgtaga
aaacctagacaaaccatccagttcagagctccattccattccagcaccagccaacagcacaaaactaatccttgacaggtgtt
cctaccaaatctgcgtcatagccaacaacagtgtgggtgcttctcctgcttctgtaatagtcatctctgcagaccccgaaaaca
aagaggttgaggaagaaagaattgcaggcacagagggtggattctctctgtcttggaaaccccaacctggagatgttatag
gctatgttgtggactgglgtgaccatacccaggatgtgctcggtgatttccagtggaagaatgtaggtcccaataccacaagc
acagtcattagcacagatgcttttaggccaggagttcgatatgacttcagaatttatgggttatctacaaaaaggattgcttgttt
attagagaaaaaaacaggatactctcaggaacttgctccttcagacaaccctcacgtgctggtggatacattgacatcccact
ccttcactctgagttggaaagattactctactgaatctcaacctggttttatacaagggtaccatgtctatctgaaatccaaggcg
aggcagtgccacccacgatttgaaaaggcagttctttcagatggttcagaatgttgcaaatacaaaattgacaacccggaag
aaaaggcattgattgtggacaacctaaagccagaatccttctatgagtttttcatcactccattcactagtgctggtgaaggccc
cagtgctacgttcacgaaggtcacgactccggatgaacactcctcgatgctgattcatatcctactgcccatggttttctgcgtc
ttgctcatcatggtcatgtgctacttgaaaagtcagtggatcaaggagacctgttatcctgacatccctgacccttacaagagc
agcatcctgtcattaataaaattcaaggagaaccctcacctaataataatgaatgtcagtgactgtatcccagatgctattgaag
ttgtaagcaagccagaagggacaaagatacagttcctaggcactaggaagtcactcacagaaaccgagttgactaagccta
actacctttatctccttccaacagaaaagaatcactctggccctggcccctgcatctgttttgagaacttgacctataaccaggc
agcttctgactctggctcttgtggccatgttccagtatccccaaaagccccaagtatgctgggactaatgacctcacctgaaaa
tgtactaaaggcactagaaaaaaactacatgaactccctgggagaaatcccagctggagaaacaagtttgaattatgtgtccc
agttggcttcacccatgtttggagacaaggacagtctcccaacaaacccagtagaggcaccacactgttcagagtataaaat
gcaaatggcagtctccctgcgtcttgccttgcctcccccgaccgagaatagcagcctctcctcaattacccttttagatccagg
tgaacactactgctaa; NP_003990.1.
(SEQ ID NO: 36)
OCT3/4(POU5F1): NIVI_203289: atgcacttctacagactattccttggggccacacgtaggttcttgaatcccg
aatggaaaggggagattgataactggtgtgtttatgttcttacaagtcttctgccttttaaaatccagtcccaggacatcaaagct
ctgcagaaagaactcgagcaatttgccaagctcctgaagcagaagaggatcaccctgggatatacacaggccgatgtggg
gctcaccctgggggttctatttgggaaggtattcagccaaacgaccatctgccgctltgaggctctgcagcttagcttcaaga
acatgtgtaagctgcggcccttgctgcagaagtgggtggaggaagctgacaacaatgaaaatcttcaggagatatgcaaag
cagaaaccctcgtgcaggcccgaaagagaaagcgaaccagtatcgagaaccgagtgagaggcaacctggagaatttgtt
cctgcagtgcccgaaacccacactgcagcagatcagccacatcgcccagcagcttgggctcgagaaggatgtggtccga
gtgtggttctgtaaccggcgccagaagggcaagcgatcaagcagcgactatgcacaacgagaggattttgaggctgctgg
gtctcctttctcagggggaccagtgtcctttcctctggccccagggccccattttggtaccccaggctatgggagccctcactt
cactgcactgtactcctcggtccctttccctgagggggaagcctttccccctgtctccgtcaccactctgggctctcccatgca
ttcaaactga;
(SEQ ID NO: 37)
NM_002701: atggcgggacacctggcttcggatttcgccttctcgccccctccaggtggtggaggtgatgggccagg
ggggccggagccgggctgggttgatcctcggacctggctaagcttccaaggccctcctggagggccaggaatcgggcc
gggggttgggccaggctctgaggtgtgggggattcccccatgccccccgccgtatgagttctgtggggggatggcgtact
gtgggccccaggttggagtggggctagtgccccaaggcggcttggagacctctcagcctgagggcgaagcaggagtcg
gggtggagagcaactccgatggggcctccccggagccctgcaccgtcacccctggtgccgtgaagctggagaaggaga
agctggagcaaaacccggaggagtcccaggacatcaaagctctgcagaaagaactcgagcaatttgccaagctcctgaag
cagaagaggatcaccctgggatatacacaggccgatgtggggctcaccctgggggttctatttgggaaggtattcagccaa
acgaccatctgccgctttgaggctctgcagcttagcttcaagaacatgtgtaagctgcggcccttgctgcagaagtgggtgg
aggaagctgacaacaatgaaaatcttcaggagatatgcaaagcagaaaccctcgtgcaggcccgaaagagaaagcgaac
cagtatcgagaaccgagtgagaggcaacctggagaatttgttcctgcagtgcccgaaacccacactgcagcagatcagcc
acatcgcccagcagcttgggctcgagaaggatgtggtccgagtgtggttctgtaaccggcgccagaagggcaagcgatca
agcagcgactatgcacaacgagaggattttgaggctgctgggtctcctttctcagggggaccagtgtcctttcctctggcccc
agggccccattttggtaccccaggctatgggagccctcacttcactgcactgtactcctcggtccctttccctgagggggaag
cctttccccctgtctccgtcaccactctgggctctcccatgcattcaaactga; NM 013633; EF032593; NM
131112; NM001114955.
(SEQ ID NO: 38)
SOX2: NM_003106: tgtacaacatgatggagacggagctgaagccgccgggcccgcagcaaacttcggggggc
ggcggcggcaactccaccgcggcggcggccggcggcaaccagaaaaacagcccggaccgcgtcaagcggcccatga
atgccttcatggtgtggtcccgcgggcagcggcgcaagatggcccaggagaaccccaagatgcacaactcggagatcag
caagcgcctgggcgccgagtggaaacttttgtcggagacggagaagcggccgttcatcgacgaggctaagcggctgcga
gcgctgcacatgaaggagcacccggattataaataccggccccggcggaaaaccaagacgctcatgaagaaggataagt
acacgctgcccggcgggctgctggcccccggcggcaatagcatggcgagcggggtcggggtgggcgccggcctggg
cgcgggcgtgaaccagcgcatggacagttacgcgcacatgaacggctggagcaacggcagctacagcatgatgcagga
ccagctgggctacccgcagcacccgggcctcaatgcgcacggcgcagcgcagatgcagcccatgcaccgctacgacgt
gagcgccctgcagtacaactccatgaccagctcgcagacctacatgaacggctcgcccacctacagcatgtcctactcgca
gcagggcacccctggcatggctcttggctccatgggttcggtggtcaagtccgaggccagctccagcccccctgtggttac
ctcttcctcccactccagggcgccctgccaggccggggacctccgggacatgatcagcatgtatctccccggcgccgagg
tgccggaacccgccgcccccagcagacttcacatgtcccagcactaccagagcggcccggtgcccggcacggccattaa
cggcacactgcccctctcacacatgtga; NM 011443; NM 00110918.
(SEQ ID NO: 39)
FGF4: NM_002007: atgtcggggcccgggacggccgcggtagcgctgctcccggcggtcctgctggccttgctgg
cgccctgggcgggccgagggggcgccgccgcacccactgcacccaacggcacgctggaggccgagctggagcgccg
ctgggagagcctggtggcgctctcgttggcgcgcctgccggtggcagcgcagcccaaggaggcggccgtccagagcg
gcgccggcgactacctgctgggcatcaagcggctgcggcggctctactgcaacgtgggcatcggcttccacctccaggc
gctccccgacggccgcatcggcggcgcgcacgcggacacccgcgacagcctgctggagctctcgcccgtggagcggg
gcgtggtgagcatcttcggcgtggccagccggttcttcgtggccatgagcagcaagggcaagctctatggctcgcccttctt
caccgatgagtgcacgttcaaggagattctccttcccaacaactacaacgcctacgagtcctacaagtaccccggcatgttca
tcgccctgagcaagaatgggaagaccaagaaggggaaccgagtgtcgcccaccatgaaggtcacccacttcctccccag
gctgtga; NM 010202; NM 004380; NM 001025432; NM 004379.2;
NP 004370; NM 134442; NP 604391.
(SEQ ID NO: 40)
Gata2: NM_032638: atggaggtggcgccggagcagccgcgctggatggcgcacccggccgtgctgaatgcgca
gcaccccgactcacaccacccgggcctggcgcacaactacatggaacccgcgcagctgctgcctccagacgaggtgga
cgtcttcttcaatcacctcgactcgcagggcaacccctactatgccaaccccgctcacgcgcgggcgcgcgtctcctacagc
cccgcgcacgcccgcctgaccggaggccagatgtgccgcccacacttgttgcacagcccgggtttgccctggctggacg
ggggcaaagcagccctctctgccgctgcggcccaccaccacaacccctggaccgtgagccccttctccaagacgccact
gcacccctcagctgctggaggccctggaggcccactctctgtgtacccaggggctgggggtgggagcgggggaggcag
cgggagctcagtggcctccctcacccctacagcagcccactctggctcccaccttttcggcttcccacccacgccacccaaa
gaagtgtctcctgaccctagcaccacgggggctgcgtctccagcctcatcttccgcggggggtagtgcagcccgaggaga
ggacaaggacggcgtcaagtaccaggtgtcactgacggagagcatgaagatggaaagtggcagtcccctgcgcccagg
cctagctactatgggcacccagcctgctacacaccaccccatccccacctacccctcctatgtgccggcggctgcccacga
ctacagcagcggactcttccaccccggaggcttcctggggggaccggcctccagcttcacccctaagcagcgcagcaag
gctcgttcctgttcagaaggccgggagtgtgtcaactgtggggccacagccacccctctctggcggcgggacggcaccgg
ccactacctgtgcaatgcctgtggcctctaccacaagatgaatgggcagaaccgaccactcatcaagcccaagcgaagact
gtcggccgccagaagagccggcacctgttgtgcaaattgtcagacgacaaccaccaccttatggcgccgaaacgccaacg
gggaccctgtctgcaacgcctgtggcctctactacaagctgcacaatgttaacaggccactgaccatgaagaaggaaggg
atccagactcggaaccggaagatgtccaacaagtccaagaagagcaagaaaggggcggagtgcttcgaggagctgtcaa
agtgcatgcaggagaagtcatcccccttcagtgcagctgccctggctggacacatggcacctgtgggccacctcccgccct
tcagccactccggacacatcctgcccactccgacgcccatccacccctcctccagcctctccttcggccacccccacccgtc
cagcatggtgaccgccatgggctag; AB 102789; AB 102790; NM 008090.
(SEQ ID NO: 41)
Gata3: NM 001002295:
atggaggtgacggcggaccagccgcgctgggtgagccaccaccaccccgccgtgctcaacgggcagcacccggacac
gcaccacccgggcctcagccactcctacatggacgcggcgcagtacccgctgccggaggaggtggatgtgctttttaacat
cgacggtcaaggcaaccacgtcccgccctactacggaaactcggtcagggccacggtgcagaggtaccctccgacccac
cacgggagccaggtgtgccgcccgcctctgcttcatggatccctaccctggctggacggcggcaaagccctgggcagcc
accacaccgcctccccctggaatctcagccccttctccaagacgtccatccaccacggctccccggggcccctctccgtcta
ccccccggcctcgtcctcctccttgtcggggggccacgccagcccgcacctcttcaccttcccgcccaccccgccgaagg
acgtctccccggacccatcgctgtccaccccaggctcggccggctcggcccggcaggacgagaaagagtgcctcaagta
ccaggtgcccctgcccgacagcatgaagctggagtcgtcccactcccgtggcagcatgaccgccctgggtggagcctcct
cgtcgacccaccaccccatcaccacctacccgccctacgtgcccgagtacagctccggactcttcccccccagcagcctgc
tgggcggctcccccaccggcttcggatgcaagtccaggcccaaggcccggtccagcacagaaggcagggagtgtgtga
actgtggggcaacctcgaccccactgtggcggcgagatggcacgggacactacctgtgcaacgcctgcgggctctatcac
aaaatgaacggacagaaccggcccctcattaagcccaagcgaaggctgtctgcagccaggagagcagggacgtcctgtg
cgaactgtcagaccaccacaaccacactctggaggaggaatgccaatggggaccctgtctgcaatgcctgtgggctctact
acaagcttcacaatattaacagacccctgactatgaagaaggaaggcatccagaccagaaaccgaaaaatgtctagcaaat
ccaaaaagtgcaaaaaagtgcatgactcactggaggacttccccaagaacagctcgtttaacccggccgccctctccagac
acatgtcctccctgagccacatctcgcccttcagccactccagccacatgctgaccacgcccacgccgatgcacccgccat
ccagcctgtcctttggaccacaccacccctccagcatggtcaccgccatgggttag; NM
008091; AM392688; AM392571; NM 001002295; NM 002051.
(SEQ ID NO: 42)
Gata4: BC101580:
atgtatcagagcttggccatggccgccaaccacgggccgccccccggtgcctacgaggcgggcggccccggcgccttc
atgcacggcgcgggcgccgcgtcctcgccagtctacgtgcccacaccgcgggtgccctcctccgtgctgggcctgtccta
cctccagggcggaggcgcgggctctgcgtccggaggcgcctcgggcggcagctccggtggggccgcgtctggtgcgg
ggcccgggacccagcagggcagcccgggatggagccaggcgggagccgacggagccgcttacaccccgccgccggt
gtcgccgcgcttctccttcccggggaccaccgggtccctggcggccgccgccgccgctgccgcggcccgggaagctgc
ggcctacagcagtggcggcggagcggcgggtgcgggcctggcgggccgcgagcagtacgggcgcgccggcttcgcg
ggctcctactccagcccctacccggcttacatggccgacgtgggcgcgtcctgggccgcagccgccgccgcctccgccg
gccccttcgacagcccggtcctgcacagcctgcccggccgggccaacccggccgcccgacaccccaatctcgatatgttt
gacgacttctcagaaggcagagagtgtgtcaactgtggggctatgtccaccccgctctggaggcgagatgggacgggtca
ctatctgtgcaacgcctgcggcctctaccacaagatgaacggcatcaaccggccgctcatcaagcctcagcgccggctgtc
cgcctcccgccgagtgggcctctcctgtgccaactgccagaccaccaccaccacgctgtggcgccgcaatgcggagggc
gagcctgtgtgcaatgcctgcggcctctacatgaagctccacggggtccccaggcctcttgcaatgcggaaagaggggat
ccaaaccagaaaacggaagcccaagaacctgaataaatctaagacaccagcagctccttcaggcagtgagagccttcctc
ccgccagcggtgcttccagcaactccagcaacgccaccaccagcagcagcgaggagatgcgtcccatcaagacggagc
ctggcctgtcatctcactacgggcacagcagctccgtgtcccagacgttctcagtcagtgcgatgtctggccatgggccctc
catccaccctgtcctctcggccctgaagctctccccacaaggctatgcgtctcccgtcagccagtctccacagaccagctcc
aagcaggactcttggaacagcctggtcttggccgacagtcacggggacataatcactgcgtaa;
AF179424; DQ666280.
(SEQ ID NO: 43)
Gata5: BC117356:
atgtaccagagcctggcgctggccgcgagcccccgccaggccgcctacgccgactcgggctccttcctgcacgctccgg
gcgccggctctccgatgtttgtgccgccggcgcgcgtcccctcgatgctgtcctacctgtccgggtgtgagccgagcccgc
agccccccgagctcgctgcgcgccccggctgggcgcagacagccaccgcggattcgtcggccttcggcccgggcagtc
cgcaccccccagccgcgcacccgcccggggccaccgccttccctttcgcgcacagcccctcggggcccggcagcggc
ggcagcgcggggggccgagacggcagtgcctaccagggcgcgctgttgcctcgagaacagttcgcggccccgcttggg
cggccggtggggacctcgtactccgccacctacccggcctacgtgagccccgacgtggcccagtcctggactgccgggc
ccttcgatggcagcgtcctgcacggcctcccaggccgcaggcccaccttcgtgtccgacttcttggaggagttcccgggtg
agggtcgtgagtgtgtcaactgcggggccctgtccacaccgctgtggcgccgagatggcaccggccactacctgtgcaat
gcctgcggcctctaccacaagatgaatggcgtcaaccggccgctcgttcggcctcagaagcgcctgtcctcgtcccgccg
cgccggcctctgctgcaccaactgccacacgaccaacaccacgctgtggcggcggaactcggagggggagcccgtgtg
caatgcctgcggcctctacatgaagctgcacggggtgccgcggcctctggctatgaagaaagaaagcatccagacacgg
aagcggaagccaaagaccatcgccaaggccaggggctcctcaggatccacaaggaatgcctcggcctccccatctgctgt
cgccagcactgacagctcagcagccacttccaaagccaagcccagcctggcgtccccagtgtgccctgggcccagcatg
gccccccaggcctctggccaggaggatgactctcttgcccccggccacttggagttcaagttcgagcctgaggactttgcct
tcccctccacggccccgagcccccaggctggcctcaggggggctctgcgccaagaggcctggtgtgcgctggccttggc
ctag; BC 105654.
(SEQ ID NO: 44)
Gata6: NM_005257: atggccttgactgacggcggctggtgcttgccgaagcgcttcggggccgcgggtgcggacg
ccagcgactccagagcctttccagcgcgggagccctccacgccgccttcccccatctcttcctcgtcctcctcctgctcccg
gggcggagagcggggccccggcggcgccagcaactgcgggacgcctcagctcgacacggaggcggcggccggacc
cccggcccgctcgctgctgctcagttcctacgcttcgcatcccttcggggctccccacggaccttcggcgcctggggtcgc
gggccccgggggcaacctgtcgagctgggaggacttgctgctgttcactgacctcgaccaagccgcgaccgccagcaag
ctgctgtggtccagccgcggcgccaagctgagccccttcgcacccgagcagccggaggagatgtaccagaccctcgccg
ctctctccagccagggtccggccgcctacgacggcgcgcccggcggcttcgtgcactctgcggccgcggcggcagcag
ccgcggcggcggccagctccccggtctacgtgcccaccacccgcgtgggttccatgctgcccggcctaccgtaccacctg
caggggtcgggcagtgggccagccaaccacgcgggcggcgcgggcgcgcaccccggctggcctcaggcctcggccg
acagccctccatacggcagcggaggcggcgcggctggcggcggggccgcggggcctggcggcgctggctcagccgc
ggcgcacgtctcggcgcgcttcccctactctcccagcccgcccatggccaacggcgccgcgcgggagccgggaggcta
cgcggcggcgggcagtgggggcgcgggaggcgtgagcggcggcggcagtagcctggcggccatgggcggccgcga
gccccagtacagctcgctgtcggccgcgcggccgctgaacgggacgtaccaccaccaccaccaccaccaccaccaccat
ccgagcccctactcgccctacgtgggggcgccactgacgcctgcctggcccgccggacccttcgagaccccggtgctgc
acagcctgcagagccgcgccggagccccgctcccggtgccccggggtcccagtgcagacctgctggaggacctgtccg
agagccgcgagtgcgtgaactgcggctccatccagacgccgctgtggcggcgggacggcaccggccactacctgtgca
acgcctgcgggctctacagcaagatgaacggcctcagccggcccctcatcaagccgcagaagcgcgtgccttcatcacg
gcggcttggattgtcctgtgccaactgtcacaccacaactaccaccttatggcgcagaaacgccgagggtgaacccgtgtg
caatgcttgtggactctacatgaaactccatggggtgcccagaccacttgctatgaaaaaagagggaattcaaaccaggaaa
cgaaaacctaagaacataaataaatcaaagacttgctctggtaatagcaataattccattcccatgactccaacttccacctctt
ctaactcagatgattgcagcaaaaatacttcccccacaacacaacctacagcctcaggggcgggtgccccggtgatgactg
gtgcgggagagagcaccaatcccgagaacagcgagctcaagtattcgggtcaagatgggctctacataggcgtcagtctc
gcctcgccggccgaagtcacgtcctccgtgcgaccggattcctggtgcgccctggccctggcctga;
AF179425; EF444980; NM 005257; NP 005248.
(SEQ ID NO: 45)
HNF1: NM_000458: atggtgtccaagctcacgtcgctccagcaagaactcctgagcgccctgctgagctccggggt
caccaaggaggtgctggttcaggccttggaggagttgctgccatccccgaacttcggggtgaagctggagacgctgcccc
tgtcccctggcagcggggccgagcccgacaccaagccggtcttccatactctcaccaacggccacgccaagggccgctt
gtccggcgacgagggctccgaggacggcgacgactatgacacacctcccatcctcaaggagctgcaggcgctcaacacc
gaggaggcggcggagcagcgggcggaggtggaccggatgctcagtgaggacccttggagggctgctaaaatgatcaa
gggttacatgcagcaacacaacatcccccagagggaggtggtcgatgtcaccggcctgaaccagtcgcacctctcccagc
atctcaacaagggcacccctatgaagacccagaagcgtgccgctctgtacacctggtacgtcagaaagcaacgagagatc
ctccgacaattcaaccagacagtccagagttctggaaatatgacagacaaaagcagtcaggatcagctgctgtttctctttcca
gagttcagtcaacagagccatgggcctgggcagtccgatgatgcctgctctgagcccaccaacaagaagatgcgccgcaa
ccggttcaaatgggggcccgcgtcccagcaaatcttgtaccaggcctacgatcggcaaaagaaccccagcaaggaagag
agagaggccttagtggaggaatgcaacagggcagaatgtttgcagcgaggggtgtccccctccaaagcccacggcctgg
gctccaacttggtcactgaggtccgtgtctacaactggtttgcaaaccgcaggaaggaggaggcattccggcaaaagctgg
ccatggacgcctatagctccaaccagactcacagcctgaaccctctgctctcccacggctccccccaccaccagcccagct
cctctcctccaaacaagctgtcaggagtgcgctacagccagcagggaaacaatgagatcacttcctcctcaacaatcagtca
ccatggcaacagcgccatggtgaccagccagtcggttttacagcaagtctccccagccagcctggacccaggccacaatct
cctctcacctgatggtaaaatgatctcagtctcaggaggaggtttgcccccagtcagcaccttgacgaatatccacagcctct
cccaccataatccccagcaatctcaaaacctcatcatgacacccctctctggagtcatggcaattgcacaaagcctcaacacc
tcccaagcacagagtgtccctgtcatcaacagtgtggccggcagcctggcagccctgcagcccgtccagttctcccagcag
ctgcacagccctcaccagcagcccctcatgcagcagagcccaggcagccacatggcccagcagcccttcatggcagctg
tgactcagctgcagaactcacacatgtacgcacacaagcaggaacccccccagtattcccacacctcccggtttccatctgc
aatggtggtcacagataccagcagcatcagtacactcaccaacatgtcttcaagtaaacagtgtcctctacaagcctggtga;
NM_013103; NM 000545; NM 009327;
(SEQ ID NO: 46)
NM 012669:
atggtttctaagttgagccagctgcagacggagctcctggctgctctgctcgagtcgggcctgagcaaagaggctctgatcc
aggctctgggggagcccgggccctacctgatggttggagatggtcccctggacaagggggagtcctgcggtgggactcg
aggggacctgaccgagctgcccaatggcctgggggagacgcgtggctcggaagatgacacggatgacgatggggaag
acttcgcgccacccattctgaaagagctggagaacctcagcccagaggaggcagcccaccagaaagccgtggtggagtc
acttcttcaggaggacccatggcgcgtggcaaagatggtcaagtcgtacctgcagcaacacaacatcccccagcgggagg
tggtggacactacgggtctcaaccagtcccacctgtcccagcacctcaacaagggcacccccatgaagacgcagaagcg
ggccgcgctgtacacctggtacgtccgcaagcagcgagaggtggctcagcaattcacccacgcggggcagggcggact
gattgaagagcccacaggtgatgagctgccaaccaaaaaggggcggaggaaccggttcaagtggggccccgcatccca
gcagatcctgttccaggcttacgagaggcagaagaaccccagcaaggaagagcgagagaccttggtggaggagtgcaat
agggcggagtgcatccagagaggggtgtcaccatcgcaggcccaggggctaggctccaaccttgtcaccgaggtgcgtg
tctacaactggtttgccaaccggcgcaaggaagaagcctttcggcataagctggccatggacacgtataacgggcctccac
ccgggccaggccccggccctgcgctacctgcccacagttccccgggcctgcccacaaccaccctctctcccagtaaggtc
cacggtgtgcggtatggacagtctgcaaccagcgaggcagctgaggtgccctccagcagcggaggtcccttagtcacagt
gtctgcggccttacaccaagtgtcccccacaggcttggagcccagcagcctgctgagcaccgaggccaagctggtctcag
ccacggggggtcccctgcctcccgtcagcaccctgacagcactgcacagcttggagcagacgtctccaggtctcaaccag
cagccgcagaaccttatcatggcctcgctgcctggggtcatgaccatcggcccaggggagcccgcctccctgggtcccac
gttcactaacacgggtgcctctaccctggtcattggtctggcctccacacaggcacagagcgtgccagtcatcaacagcatg
gggagcagcctgaccaccctgcagccggtccagttttcccagccactgcacccttcctatcagcagcctctcatgccccctg
tacagagccacgtggcccagagtcccttcatggcaaccatggcccagctgcagagcccccacgccctgtacagccacaa
gcctgaggtggcccagtacacgcatacaagcctgcttccgcagaccatgctgatcacagacaccaacctcagcacccttgc
cagcctcacgcccaccaagcaggtcttcacctcagacacagaggcctccagtgagcctgggcttcatgagccgtcgtctcc
agccacaaccattcacatccccagccaggacccgtcaaacatccagcacctgcagcctgctcaccggctcagcaccagtc
ccacagtgtcctccagcagcctggtgttgtaccagagttctgactccaacgggcacagccacctgctgccatccaaccacg
gtgtcatcgagacttttatctccacccagatggcctcctcctcccagtaa; NM_009330.
(SEQ ID NO: 47)
HNF3: X74936: atgttagggactgtgaagatggaagggcatgagagcaacgactggaacagctactacgcggacac
gcaggaggcctactcctctgtccctgtcagcaacatgaactccggcctgggctctatgaactccatgaacacctacatgacc
atgaacaccatgaccacgagcggcaacatgaccccggcttccttcaacatgtcctacgccaacacgggcttaggggccgg
cctgagtcccggtgctgtggctggcatgccaggggcctctgcaggcgccatgaacagcatgactgcggcgggcgtcacg
gccatgggtacggcgctgagcccgggaggcatgggctccatgggcgcgcagcccgtcacctccatgaacggcctgggt
ccctacgccgccgccatgaacccgtgcatgagtcccatggcgtacgcgccgtccaacctgggccgcagccgcgcgggg
ggcggcggcgacgccaagacattcaagcgcagctaccctcacgccaagccgccttactcctacatctcgctcatcacgatg
gccatccagcaggcgcccagcaagatgctcacgctgagcgagatctaccagtggatcatggacctcttcccctattaccgc
cagaaccagcagcgctggcagaactccatccgccactcgctgtccttcaacgattgtttcgtcaaggtggcacgatccccag
acaagccaggcaagggctcctactggacgctgcacccggactccggcaacatgttcgagaacggctgctacttgcgccgc
caaaagcgcttcaagtgtgagaagcagccgggggccggaggtgggagtgggggcggcggctccaaagggggcccag
aaagtcgcaaggacccctcaggcccggggaaccccagcgccgagtcaccccttcattggggtgtgcacggaaaggctag
ccagctagagggcgcgccggcccccgggcccgccgccagcccccagactctggaccacagcggggccacggcgaca
gggggcgcttcggagttgaagtctccagcgtcttcatctgcgccccccataagctccgggccaggggcgctggcatctgta
cccccctctcacccggctcacggcctggcaccccacgaatctcagctgcatctgaaaggggatccccactactcctttaatc
accccttctccatcaacaacctcatgtcctcctccgagcaacagcacaagctggacttcaaggcatacgagcaggcgctgc
agtactctccttatggcgctaccttgcccgccagtctgccccttggcagcgcctcagtggccacgaggagccccatcgagc
cctcagccctggagccagcctactaccaaggtgtgtattccagacccgtgctaaatacttcctag;
(SEQ ID NO: 48)
HNF3gammaX74938M: atgctgggctcagtgaagatggaggctcatgacctggccgagtggagctactacccg
gaggcgggcgaggtgtattctccagtgaatcctgtgcccaccatggcccctctcaactcctacatgaccttgaacccactca
gctctccctaccctcccggagggcttcaggcctccccactgcctacaggacccctggcacccccagcccccactgcgccct
tggggcccaccttcccaagcttgggcactggtggcagcaccggaggcagtgcttccgggtatgtagccccagggcccgg
gcttgtacatggaaaagagatggcaaaggggtaccggcggccactggcccacgccaaaccaccatattcctacatctctct
cataaccatggctattcagcaggctccaggcaagatgctgaccctgagtgaaatctaccaatggatcatggacctcttcccgt
actaccgggagaaccagcaacgttggcagaactccatccggcattcgctgtccttcaatgactgcttcgtcaaggtggcacg
ctccccagacaagccaggcaaaggctcctactgggccttgcatcccagctctgggaacatgtttgagaacggatgctatctc
cgccggcagaagcgcttcaagctggaggagaaggcaaagaaaggaaacagcgccacatcggccagcaggaatggtac
tgcggggtcagccacctctgccaccactacagctgccactgcagtcacctccccggctcagccccagcctacgccatctga
gcccgaggcccagagtggggatgatgtggggggtctggactgcgcctcacctccttcgtccacaccttatttcagcggcct
ggagctcccgggggaactaaagttggatgcgccctataacttcaaccaccctttctctatcaacaacctgatgtcagaacaga
catcgacaccttccaaactggatgtggggtttgggggctacggggctgagagtggggagcctggagtctactaccagagc
ctctattcccgctctctgcttaatgcatcctag;
(SEQ ID NO: 49)
HNF3betaX74937: atgctgggagccgtgaagatggaagggctcgagccatccgactggagcagctactacgcgg
agcccgagggctactcttccgtgagcaacatgaacgccggcctggggatgaatggcatgaacacatacatgagcatgtcc
gcggctgccatgggcggcggttccggcaacatgagcgcgggctccatgaacatgtcatcctatgtgggcgctggaatgag
cccgtcgctagctggcatgtccccgggcgccggcgccatggcgggcatgagcggctcagccggggcggccggcgtgg
cgggcatgggacctcacctgagtccgagtctgagcccgctcgggggacaggcggccggggccatgggtggccttgccc
cctacgccaacatgaactcgatgagccccatgtacgggcaggccggcctgagccgcgctcgggaccccaagacataccg
acgcagctacacacacgccaaacctccctactcgtacatctcgctcatcaccatggccatccagcagagccccaacaagat
gctgacgctgagcgagatctatcagtggatcatggacctcttccctttctaccggcagaaccagcagcgctggcagaactcc
atccgccactctctctccttcaacgactgctttctcaaggtgccccgctcgccagacaagcctggcaagggctccttctggac
cctgcacccagactcgggcaacatgttcgagaacggctgctacctgcgccgccagaagcgcttcaagtgtgagaagcaac
tggcactgaaggaagccgcgggtgcggccagtagcggaggcaagaagaccgctcctgggtcccaggcctctcaggctc
agctcggggaggccgcgggctcggcctccgagactccggcgggcaccgagtccccccattccagcgcttctccgtgtca
ggagcacaagcgaggtggcctaagcgagctaaagggagcacctgcctctgcgctgagtcctcccgagccggcgccctc
gcctgggcagcagcagcaggctgcagcccacctgctgggcccacctcaccacccaggcctgccaccagaggcccacct
gaagcccgagcaccattacgccttcaaccaccccttctctatcaacaacctcatgtcgtccgagcagcaacatcaccacagc
caccaccaccatcagccccacaaaatggacctcaaggcctacgaacaggtcatgcactacccagggggctatggttcccc
catgccaggcagcttggccatgggcccagtcacgaacaaagcgggcctggatgcctcgcccctggctgcagacacttcct
actaccaaggagtgtactccaggcctattatgaactcatcctaa;
(SEQ ID NO: 50)
HNF3G: AH008133: atgctgggctcagtgaagatggaggcccatgacctggccgagtggagctactacccggag
gcgggcgaggtctactcgccggtgaccccagtgcccaccatggcccccctcaactcctacatgaccctgaatcctctaagc
tctccctatccccctggggggctccctgcctccccactgccctcaggacccctggcacccccagcacctgcagcccccctg
gggcccactttcccaggcctgggtgtcagcggtggcagcagcagctccgggtacggggccccgggtcctgggctggtgc
acgggaaggagatgccgaaggggtatcggcggcccctggcacacgccaagccaccgtattcctatatctcactcatcacc
atggccatccagcaggcgccgggcaagatgctgaccttgagtgaaatctaccagtggatcatggacctcttcccttactacc
gggagaatcagcagcgctggcagaactccattcgccactcgctgtctttcaacgactgcttcgtcaaggtggcgcgttcccc
agacaagcctggcaagggctcctactgggccctacaccccagctcagggaacatgtttgagaatggctgctacctgcgcc
gccagaaacgcttcaagctggaggagaaggtgaaaaaagggggcagcggggctgccaccaccaccaggaacgggac
agggtctgctgcctcgaccaccacccccgcggccacagtcacctccccgccccagcccccgcctccagcccctgagcct
gaggcccagggcggggaagatgtgggggctctggactgtggctcacccgcttcctccacaccctatttcactggcctgga
gctcccaggggagctgaagctggacgcgccctacaacttcaaccaccctttctccatcaacaacctaatgtcagaacagac
accagcacctcccaaactggacgtggggtttgggggctacggggctgaaggtggggagcctggagtctactaccagggc
ctctattcccgctctttgcttaatgcatcctag;
(SEQ ID NO: 51)
HNF3A: AH008132: atgttaggaactgtgaagatggaagggcatgaaaccagcgactggaacagctactacgcag
acacgcaggaggcctactcctcggtcccggtcagcaacatgaactcaggcctgggctccatgaactccatgaacacctaca
tgaccatgaacaccatgactacgagcggcaacatgaccccggcgtccttcaacatgtcctatgccaacccggccttagggg
ccggcctgagtcccggcgcagtagccggcatgccggggggctcggcgggcgccatgaacagcatgactgcggccggc
gtgacggccatgggtacggcgctgagcccgagcggcatgggcgccatgggtgcgcagcaggcggcctccatgatgaat
ggcctgggcccctacgcggccgccatgaacccgtgcatgagccccatggcgtacgcgccgtccaacctgggccgcagc
cgcgcgggcggcggcggcgacgccaagacgttcaagcgcagttacccgcacgccaagccgccctactcgtacatctcg
ctcatcaccatggccatccagcgggcgcccagcaagatgctcacgctgagcgagatctaccagtggatcatggacctcttc
ccctattaccggcagaaccagcagcgctggcagaactccatccgccactcgctgtccttcaatgactgcttcgtcaaggtgg
cacgctccccggacaagccgggcaagggctcctactggacgctgcacccggactccggcaacatgttcgagaacggctg
ctacttgcgccgccagaagcgcttcaagtgcgagaagcagccgggggccggcggcgggggcgggagcggaagcggg
ggcagcggcgccaagggcggccctgagagccgcaaggacccctctggcgcctctaaccccagcgccgactcgcccctc
catcggggtgtgcacgggaagaccggccagctagagggcgcgccggccccgggcccggccgccagcccccagactct
ggaccacagtggggcgacggcgacagggggcgcctcggagttgaagactccagcctcctcaactgcgccccccataag
ctccgggcccggggcgctggcctctgtgcccgcctctcacccggcacacggcttggcaccccacgagtcccagctgcac
ctgaaaggggacccccactactccttcaaccacccgttctccatcaacaacctcatgtcctcctcggagcagcagcataagc
tggacttcaaggcatacgaacaggcactgcaatactcgccttacggctctacgttgcccgccagcctgcctctaggcagcg
cctcggtgaccaccaggagccccatcgagccctcagccctggagccggcgtactaccaaggtgtgtattccagacccgtc
ctaaacacttcctag;
(SEQ ID NO: 52)
HNF4alpha: NM_008261: atgcgactctctaaaacccttgccggcatggatatggccgactacagcgctgccctg
gacccagcctacaccaccctggagtttgaaaatgtgcaggtgttgaccatgggcaatgacacgtccccatctgaaggtgcc
aacctcaattcatccaacagcctgggcgtcagtgccctgtgcgccatclgtggcgaccgggccaccggcaaacactacgg
agcctcgagctgtgacggctgcaaggggttcttcaggaggagcgtgaggaagaaccacatgtactcctgcaggtttagcc
gacaalgtgtggtagacaaagataagaggaaccagtgtcgttactgcaggcttaagaagtgcttccgggctggcatgaaga
aggaagclgtccaaaatgagcgggaccggatcagcacgcggaggtcaagctacgaggacagcagcctgccctccatca
acgcgctcclgcaggcagaggttctgtcccagcagatcacctctcccalctclgggatcaatggcgacattcgggcaaaga
agattgccaacatcacagacgtgtgtgagtctatgaaggagcagctgctggtcctggtcgagtgggccaagtacalcccgg
ccttctgcgaactccttctggatgaccaggtggcgctgctcagggcccacgccggtgagcatctgctgcttggagccacca
agaggtccatggtgtttaaggacgtgctgctcctaggcaatgactacatcgtccctcggcactgtccagagctagcggagat
gagccgtgtgtccatccgcatcctcgatgagctggtcctgccctlccaagagctgcagattgatgacaatgaatatgcctgcc
tcaaagccalcatcttctttgatccagatgccaaggggctgagtgacccgggcaagatcaagcggctgcggtcacaggtgc
aagtgagcctggaggattacalcaacgaccggcagtacgactctcggggccgctttggagagctgctgctgctgttgccca
cgctgcagagcatcacctggcagatgatcgaacagatccagttcatcaagctcttcggcatggccaagattgacaacctgct
gcaggagatgcttctcggaggglctgccagtgatgcaccccacacccaccaccccctgcaccctcacctgatgcaagaac
acatgggcaccaatgtcattgttgctaacacgatgccctctcacctcagcaatggacagatgtgtgaglggccccgacccag
ggggcaggcagccactcccgagactccacagccatcaccaccaagtggctcgggatctgaatcclacaagctcctgccag
gagccatcaccaccatcgtcaagcclccctctgccattccccagccaacgatcaccaagcaagaagccatctag;
(SEQ ID NO: 53)
I1NF4a: NM022180: atggacatggctgactacagtgctgccttggacccagcclacaccaccctggagtttgaaaat
gtgcaggtgttgaccatgggcaatgacacatccccatctgaaggtgccaacctcaactcatccaacagcctgggtgtcagtg
ccctgtgtgccatctgtggcgatcgggccactggcaaacactacggagcctcaagctglgacggclgcaagggattcltca
ggaggagcgtgaggaagaaccacatgtactcctgcaggtttagcaggcagtgcgtggtagacaaagalaagaggaacca
gtgtcgttactgcaggctcaagaagtgcttccgggctggcatgaagaaagaagccgtccaaaatgagcgggatcggatca
gcacgcggaggtcaagctacgaggacagcagcctaccctccattaatgcgctcctgcaggcagaggtcctgtctcagcag
atcacctcccccatctctgggatcaatggcgacattcgggccaagaagattgccaacatcacggatglgtgtgagtctatgaa
ggagcagctgctggttctgglcgaatgggccaagtacatcccggccttctgtgaacttcttctggatgaccaggtggcgclg
clcagagcccacgctggtgagcacctgctgctlggagccaccaagaggtccatggtgttcaaggatglgctgctcclaggc
aatgactacatcgtccctcggcactgtccagagctagcagagatgagccgtgtgtccattcgcatcctcgatgagctggtctt
gcccttccaagagctgcagalcgatgataatgaatacgcctgcctcaaagccatcatcttctltgacccagatgccaagggg
ctgagtgacccaggcaagatcaagcggctgcggtcacaggtgcaggtgagcctggaggattacatcaacgaccggcagt
atgactctcggggtcgttltggagagctgctgctgctcctgcccactctgcagagcattacclggcagatgatcgagcagatc
cagttcatcaagctctttggcatggccaagattgacaacctgctgcaggagatgctgcttggagggtctgccagtgacgcgc
cccacgcccaccaccccctgcaccctcacctgatgcaagaacacatgggcaccaatgtcatagttgccaacacgatgccct
ctcacctcagcaatggacagatgtgtgagtggccccggcccagggggcaggcagccacccctgagactccacagccatc
accaccaagtggctctggatctgaatcctacaagctcctgccaggagccatcaccaccatcgtcaagcctccctctgccatc
ccccagccaacgatcaccaagcaggaagccatclag;
(SEQ ID NO: 54)
HNF6: U95945: atgaacgcacagctgaccatggaggcgatcggcgagctgcacggggtgagccatgagccggtgc
ccgcccctgctgacctgctgggcggcagccctcacgcgcgcagctccgtgggacaccgcggcagccacctgcctcccg
cgcacccgcgttccatgggcatggcgtccctgctggacggcggcagcggaggcagcgattaccaccaccaccaccgcg
cccctgagcacagcttggctggccccctgcaccccaccatgaccatggcctgtgaaactcccccaggtatgagcatgccca
ccacctacactaccttaacccclclgcagccgclgccgcccatctccaccgtgtccgacaagttccctcaccatcatcaccac
caccatcaccaccaccacccacaccaccaccagcgcctggcgggcaacgtgagcggtagtttcacacttatgcgggatga
gcgcgggctggcctctatgaataacctctataccccctaccacaaggacgtggctggcatgggccagagcctctcgcccct
ctctggctccggtctgggcagcattcacaactcccagcaaggacttccccactatgctcatcccggcgcggctatgcccacc
gacaagatgctcaccccaaatggctttgaagcccaccaccctgccatgctcggtcgccacggggagcagcacctcacgcc
cacctcggccggcatggtacccatcaacggccttcctccgcaccatcctcatgcccacctgaatgcccagggccacggac
agctcctgggcacagcccgagagcccaacccttcggtgaccggcgcgcaggtcagcaatggaagtaattcagggcagat
ggaagagatcaataccaaagaggtggcgcagcgtatcaccaccgagctcaaacgttacagcatcccacaggccatcttcg
cgcagagggtgctctgccgttcccaggggaccctttcggacctgctgcgaaaccccaagccctggagcaaactcaagtcg
ggtcgggagaccttccggaggatgtggaagtggctgcaggagccggagttccagcgcatgtcggcgctccgcttagcag
cctgcaaacggaaagagcaagaacatgggaaggacagaggcaacacccccaaaaagcccaggctggtcttcacagacg
tccaacgtcgaactctacatgcaatattcaaggaaaataagcgtccgtccaaagaattacaaatcaccatctcccagcagctg
gggttggagctgagcactgtcagcaacttcttcatgaatgccagaaggaggagtctggacaagtggcaggacgagggcg
gctccaactcaggcagttcatcgtcctcatcgagcacttgtaccaaagcatga;
(SEQ ID NO: 55)
IILXB9: NM001096823:
atggagaagtccaagaatttcaggattgacgctctcctggcgatagatccccccaaggctcagacctccccattggctctggt
cacctcgctgtcctcctcgtctctctccgggagccccccgtccgagcacactgacagcctcaggactgactccccctcccct
ccaaggacttgtggactggtccctaaaccaggtttcctgagcagccaccagcaccccccaaacatgatgtcattgcaccccc
aggctgctccagggatcccccctcaggccctgtatggacacccgatgtacagctacttggcagcggggcagcacccagct
ctgtcctacccctactcccagatgcagagcagccaccacccccaccccatggaccccatcaagatcagcgctggcaccttc
caactggaccagtggctcagagcctccactgccggcatgatgctgcccaaaatggcagactttaactcccaggcccaatcc
aacctgctgggaaagtgcagaagaccaaggacagcgtttaccagtcagcagctgttggaactggagcaccaattcaagct
gaacaagtacctctccaggccgaaacgctttgaagtggccacttccctgatgctcactgagacgcaggtgaagatctggttc
cagaacaggcgcatgaaatggaagaggagtaagaaagccaaggagcaggcggcgcaggactcagcagagaaacagc
agagggcaggcaagggcagcagcgaggagaagtgctcggatgagctgcaggaagagaagaaatcctaccatctccatc
ccaggggggagcccatcaaagggaacggccgcctgcagcccagagactatacagacagcgaagaggacgaggagga
ggacagggaagaggaggaagaggaagatcacagaggggaggggaagcggttttaccatcattcttctgactgcacatcc
gaggaagaggagaacagccacaataagcagagcggccactga;
(SEQ ID NO: 56)
NM 019944.
atggaaaaatccaaaaatttccgcatcgacgccctgctggccgtggatcccccgcgagccgcctccacgcagagcgcgcc
tctggccttggtcacttccctcgcgactacagtatctggtcccggccgcggcggcagcggcggcggggggaccagtagc
ggggcgagccgtagctgcagtcccgcatcctcggaggccactgcagcgcccggtgaccggctgagagctgagagcccg
tcgcccccacgcttgctggctgcacactgcgcgctgctgcccaagcccggattcctgggcgccggaggaggcggcggc
gcggcgggtgggccgggcactccccaccaccacgcgcaccctggtgcagcagccgccgcggctgccgctgccgctgc
cgcggctgccggtggcctggcactggggctgcacccggggggcgcacagggcggcgcgggcctccctgcacaggcg
gctctctatggacacccggtctacagttattcggcagcagctgcagcggccgcgctagctggccagcacccggcgctttcc
tactcataccctcaggtgcagggcgcgcaccctgcgcaccctgccgaccccatcaagctgggtgccagcaccttccaactg
gaccagtggctgcgcgcgtctactgcgggcatgatcctgcccaagatgccggacttcagctgtcaggcgcagtcgaacct
cttggggaagtgccgaaggcctcgcacggccttcaccagccagcagctgttggagctggaacaccagttcaagctcaaca
agtacctgtctcgacccaagcgttttgaggtggctacctcgctcatgctcaccgagactcaggtgaagatttggttccagaac
cgccgaatgaaatggaaacgcagcaaaaaggccaaagagcaggctgcgcaggaggcggagaagcagaagggcggc
ggcgggggcaccggcaaaggcggcagtgaggagaagacggaagaggagctgatggggcctccggtttcgggggaca
aggcaagcggccgtcgcctgcgggacttgcgggacagtgaccctgatgaggacgaggatgatgaagaagaggacaact
tcccgtacagcaatggtgccggtgcccatgctgcctcatccgactgctcatctgaggacgactcgcctcctccaagactagg
cgggcctggacaccaacctctgccccagtag;
(SEQ ID NO: 57)
NM_005515: atggaaaaatccaaaaatttccgcatcgacgccctgctggccgtggatcccccgcgagccgcctccac
gcagagcgcgcctctggccttggtcacttccctcgcgactacagtatctggtcccggccgcggcggcagcggcggcggg
gggaccagtagcggggcgagccgtagctgcagtcccgcatcctcggaggccactgcagcgcccggtgaccggctgaga
gctgagagcccgtcgcccccacgcttgctggctgcacactgcgcgctgctgcccaagcccggattcctgggcgccggag
gaggcggcggcgcggcgggtgggccgggcactccccaccaccacgcgcaccctggtgcagcagccgccgcggctgc
cgctgccgctgccgcggctgccggtggcctggcactggggctgcacccggggggcgcacagggcggcgcgggcctcc
ctgcacaggcggctctctatggacacccggtctacagttattcggcagcagctgcagcggccgcgctagctggccagcac
ccggcgctttcctactcataccctcaggtgcagggcgcgcaccctgcgcaccctgccgaccccatcaagctgggtgccag
caccttccaactggaccagtggctgcgcgcgtctactgcgggcatgatcctgcccaagatgccggacttcagctgtcaggc
gcagtcgaacctcttggggaagtgccgaaggcctcgcacggccttcaccagccagcagctgttggagctggaacaccagt
tcaagctcaacaagtacctgtctcgacccaagcgttttgaggtggctacctcgctcatgctcaccgagactcaggtgaagatt
tggttccagaaccgccgaatgaaatggaaacgcagcaaaaaggccaaagagcaggctgcgcaggaggcggagaagca
gaagggcggcggcgggggcaccggcaaaggcggcagtgaggagaagacggaagaggagctgatggggcctccggt
ttcgggggacaaggcaagcggccgtcgcctgcgggacttgcgggacagtgaccctgatgaggacgaggatgatgaaga
agaggacaacttcccgtacagcaatggtgccggtgcccatgctgcctcatccgactgctcatctgaggacgactcgcctcct
ccaagactaggcgggcctggacaccaacctctgcc;
(SEQ ID NO: 58)
Lbx1: NM_006562:
atgacttccaaggaggacggcaaggcggcgccgggggaggagcggcggcgcagcccgctggaccacctgcctccgc
ctgccaactccaacaagccactgacgccgttcagcatcgaggacatcctcaacaagccgtctgtgcggagaagttactcgc
tgtgcggggcggcgcacctgctggccgccgcggacaagcacgcgcagggcggcttgcccctggcgggccgcgcgctg
ctctcgcagacctcgccgctgtgcgcgctggaggagctcgccagcaagacgtttaaggggctggaggtcagcgttctgca
ggcagccgaaggccgcgacggtatgaccatctttgggcagcggcagacccctaagaagcggcgaaagtcgcgcacgg
ccttcaccaaccaccagatctatgaattggaaaagcgctttctataccagaagtacctgtcccccgccgatcgcgaccaaatc
gcgcagcagctgggcctcaccaacgcgcaagtcatcacctggttccagaatcggcgcgctaagctcaagcgggacctgg
aggagatgaaggccgacgtagagtccgccaagaaactgggccccagcgggcagatggacatcgtggcgctggccgaa
ctcgagcagaactcggaggccacagccggcggtggcggcggctgcggcagggccaagtcgaggcccggctctccggt
cctccccccaggcgccccgaaggccccgggcgctggcgccctgcagctctcgcctgcctctccgctcacggaccagccg
gccagcagccaggactgctcggaggacgaggaagacgaagagatcgacgtggacgattga; NM_010691.
(SEQ ID NO: 59)
Lmx1b:
atgttggacggcatcaagatggaggagcacgccctgcgccccgggcccgccactctgggggtgctgctgggctccgact
gcccgcatcccgccgtctgcgagggctgccagcggcccatctccgaccgcttcctgatgcgagtcaacgagtcgtcctgg
cacgaggagtgtttgcagtgcgcggcgtgtcagcaagccctcaccaccagctgctacttccgggatcggaaactgtactgc
aaacaagactaccaacagctcttcgcggccaagtgcagcggctgcatggagaagatcgcccccaccgagttcgtgatgcg
ggcgctggagtgcgtgtaccacctgggctgcttctgctgctgcgtgtgtgaacggcagctacgcaagggcgacgaattcgt
gctcaaggagggccagctgctgtgcaagggtgactacgagaaggagaaggacctgctcagctccgtgagccccgacga
gtccgactccgtgaagagcgaggatgaagatggggacatgaagccggccaaggggcagggcagtcagagcaagggca
gcggggatgacgggaaggacccgcggaggcccaagcgaccccggaccatcctcaccacgcagcagcgaagagccttc
aaggcctccttcgaggtctcgtcgaagccttgccgaaaggtccgagagacactggcagctgagacgggcctcagtgtgcg
cgtggtccaggtctggtttcagaaccaaagagcaaagatgaagaagctggcgcggcggcaccagcagcagcaggagca
gcagaactcccagcggctgggccaggaggtcctgtccagccgcatggagggcatgatggcttcctacacgccgctggcc
ccaccacagcagcagatcgtggccatggaacagagcccctacggcagcagcgaccccttccagcagggcctcacgccg
ccccaaatgccagggaacgactccatcttccatgacatcgacagcgatacctccttaaccagcctcagcgactgcttcctcg
gctcctcagacgtgggctccctgcaggcccgcgtggggaaccccatcgaccggctctactccatgcagagttcctacttcg
cctcctga; NM 010725.
(SEQ ID NO: 60)
Neurogenin(NEUROG1): NM_006161:
atgccagcccgccttgagacctgcatctccgacctcgactgcgccagcagcagcggcagtgacctatccggcttcctcacc
gacgaggaagactgtgccagactccaacaggcagcctccgcttcggggccgcccgcgccggcccgcaggggcgcgcc
caatatctcccgggcgtctgaggttccaggggcacaggacgacgagcaggagaggcggcggcgccgcggccggacgc
gggtccgctccgaggcgctgctgcactcgctgcgcaggagccggcgcgtcaaggccaacgatcgcgagcgcaaccgc
atgcacaacttgaacgcggccctggacgcactgcgcagcgtgctgccctcgttccccgacgacaccaagctcaccaaaat
cgagacgctgcgcttcgcctacaactacatctgggctctggccgagacactgcgcctggcggatcaagggctgcccggag
gcggtgcccgggagcgcctcctgccgccgcagtgcgtcccctgcctgcccggtcccccaagccccgccagcgacgcgg
agtcctggggctcaggtgccgccgccgcctccccgctctctgaccccagtagcccagccgcctccgaagacttcacctacc
gccccggcgaccctgttttctccttcccaagcctgcccaaagacttgctccacacaacgccctgtttcattccttaccactag;
BQ169355.
(SEQ ID NO: 61)
Neurogenin2(NEUROG2): NM024019: atgttcgtcaaatccgagaccttggagttgaaggaggaagagga
cgtgttagtgctgctcggatcggcctcccccgccttggcggccctgaccccgctgtcatccagcgccgacgaagaagagg
aggaggagccgggcgcgtcaggcggggcgcgtcggcagcgcggggctgaggccgggcagggggcgcggggcggc
gtggctgcgggtgcggagggctgccggcccgcacggctgctgggtctggtacacgattgcaaacggcgcccttcccggg
cgcgggccgtctcccgaggcgccaagacggccgagacggtgcagcgcatcaagaagacccgtagactgaaggccaac
aaccgcgagcgaaaccgcatgcacaacctcaacgcggcactggacgcgctgcgcgaggtgctccccacgttccccgag
gacgccaagctcaccaagatcgagaccctgcgcttcgcccacaactacatctgggcactcaccgagaccctgcgcctggc
ggatcactgcgggggcggcggcgggggcctgccgggggcgctcttctccgaggcagtgtlgctgagcccgggaggcg
ccagcgccgccctgagcagcagcggagacagcccctcgcccgcctccacgtggagttgcaccaacagccccgcgccgt
cctcctccgtglcctccaattccacctccccctacagctgcactttatcgcccgccagcccggccgggtcagacatggactat
tggcagcccccacctcccgacaagcaccgctatgcacctcacctccccatagccagggattgtatctag; DR001447.
(SEQ ID NO: 62)
Neurogenin3(NEUROG3): atgacgcctcaaccctcgggtgcgcccactgtccaagtgacccgtgagacggagc
ggtccttccccagagcctcggaagacgaagtgacctgccccacgtccgccccgcccagccccactcgcacacggggga
actgcgcagaggcggaagagggaggctgccgaggggccccgaggaagctccgggcacggcgcgggggacgcagcc
ggcctaagagcgagttggcactgagcaagcagcgacggagtcggcgaaagaaggccaacgaccgcgagcgcaatcga
atgcacaacctcaactcggcactggacgccctgcgcggtgtcctgcccaccttcccagacgacgcgaagctcaccaagat
cgagacgctgcgcttcgcccacaactacatctgggcgctgactcaaacgctgcgcatagcggaccacagcttgtacgcgct
ggagccgccggcgccgcactgcggggagctgggcagcccaggcggttcccccggggactgggggtccctctactccc
cagtctcccaggctggcagcctgagtcccgccgcgtcgctggaggagcgacccgggctgctgggggccaccttttccgc
ctgcttgagcccaggcagtctggctttctcagattttctgtga; NM 009719.
(SEQ ID NO: 63)
MASH1: NM_004316:
atggaaagctctgccaagatggagagcggcggcgccggccagcagccccagccgcagccccagcagcccttcctgcc
gcccgcagcctgtttctttgccacggccgcagccgcggcggccgcagccgccgcagcggcagcgcagagcgcgcagc
agcagcagcagcagcagcagcagcagcagcaggcgccgcagctgagaccggcggccgacggccagccctcagggg
gcggtcacaagtcagcgcccaagcaagtcaagcgacagcgctcgtcttcgcccgaactgatgcgctgcaaacgccggct
caacttcagcggctttggctacagcctgccgcagcagcagccggccgccgtggcgcgccgcaacgagcgcgagcgcaa
ccgcgtcaagttggtcaacctgggctttgccacccttcgggagcacgtccccaacggcgcggccaacaagaagatgagta
aggtggagacactgcgctcggcggtcgagtacatccgcgcgctgcagcagctgctggacgagcatgacgcggtgagcg
ccgccttccaggcaggcgtcctgtcgcccaccatctcccccaactactccaacgacttgaactccatggccggctcgccggt
ctcatcctactcgtcggacgagggctcttacgacccgctcagccccgaggagcaggagcttctcgacttcaccaactggttc
tga; NM 008553.
(SEQ ID NO: 64)
MyoD: NM010866: atggagcttctatcgccgccactccgggacatagacttgacaggccccgacggctctctctgct
cctttgagacagcagacgacttctatgatgacccgtgtttcgactcaccagacctgcgcttttttgaggacctggacccgcgc
ctggtgcacatgggagccctcctgaaaccggaggagcacgcacacttccctactgcggtgcacccaggcccaggcgctc
gtgaggatgagcatgtgcgcgcgcccagcgggcaccaccaggcgggtcgctgcttgctgtgggcctgcaaggcgtgca
agcgcaagaccaccaacgctgatcgccgcaaggccgccaccatgcgcgagcgccgccgcctgagcaaagtgaatgag
gccttcgagacgctcaagcgctgcacgtccagcaacccgaaccagcggctacccaaggtggagatcctgcgcaacgcca
tccgctacatcgaaggtctgcaggctctgctgcgcgaccaggacgccgcgccccctggcgccgctgccttctacgcacct
ggaccgctgcccccaggccgtggcagcgagcactacagtggcgactcagatgcatccagcccgcgctccaactgctctg
atggcatgatggattacagcggccccccaagcggcccccggcggcagaatggctacgacaccgcctactacagtgaggc
ggcgcgcgagtccaggccagggaagagtgcggctgtgtcgagcctcgactgcctgtccagcatagtggagcgcatctcc
acagacagccccgctgcgcctgcgctgcttttggcagatgcaccaccagagtcgcctccgggtccgccagagggggcat
ccctaagcgacacagaacagggaacccagaccccgtctcccgacgccgcccctcagtgtcctgcaggctcaaaccccaa
tgcgatttatcaggtgctttga;
(SEQ ID NO: 65)
NM002478: atggagctactgtcgccaccgctccgcgacgtagacctgacggcccccgacggctctctctgctcctttg
ccacaacggacgacttctatgacgacccgtgtttcgactccccggacctgcgcttcttcgaagacctggacccgcgcctgat
gcacgtgggcgcgctcctgaaacccgaagagcactcgcacttccccgcggcggtgcacccggccccgggcgcacgtga
ggacgagcatgtgcgcgcgcccagcgggcaccaccaggcgggccgctgcctactgtgggcctgcaaggcgtgcaagc
gcaagaccaccaacgccgaccgccgcaaggccgccaccatgcgcgagcggcgccgcctgagcaaagtaaatgaggcc
tttgagacactcaagcgctgcacgtcgagcaatccaaaccagcggttgcccaaggtggagatcctgcgcaacgccatccg
ctatatcgagggcctgcaggctctgctgcgcgaccaggacgccgcgccccctggcgccgcagccgccttctatgcgccg
ggcccgctgcccccgggccgcggcggcgagcactacagcggcgactccgacgcgtccagcccgcgctccaactgctc
cgacggcatgatggactacagcggccccccgagcggcgcccggcggcggaactgctacgaaggcgcctaclacaacg
agactgcccgccctcctgctggcggacgtgccttctgagtcgcctccgcgcaggcaagaggctgccgcccccagcgagg
gagagagcagcggcgaccccacccagtcaccggacgccgccccgcagtgccctgcgggtgcgaaccccaacccgata
taccaggtgctctga.
(SEQ ID NO: 66)
Myf5: NM_005593: atggacgtgatggatggctgccagttctcaccttctgagtacttctacgacggctcctgcatacc
gtcccccgagggtgaatttggggacgagtttgtgccgcgagtggctgccttcggagcgcacaaagcagagctgcagggct
cagatgaggacgagcacgtgcgagcgcctaccggccaccaccaggctggtcactgcctcatgtgggcctgcaaagcctg
caagaggaagtccaccaccatggatcggcggaaggcagccactatgcgcgagcggaggcgcctgaagaaggtcaacc
aggctttcgaaaccctcaagaggtgtaccacgaccaaccccaaccagaggctgcccaaggtggagatcctcaggaatgcc
atccgctacatcgagagcctgcaggagttgctgagagagcaggtggagaactactatagcctgccgggacagagctgctc
ggagcccaccagccccacctccaactgctctgatggcatgcccgaatgtaacagtcctgtctggtccagaaagagcagtac
ttttgacagcatctactgtcctgatgtatcaaatgtatatgccacagataaaaactccttatccagcttggattgcttatccaacat
agtggaccggatcacctcctcagagcaacctgggttgcctctccaggatctggcttctctctctccagttgccagcaccgatt
cacagcctgcaactccaggggcttctagttccaggcttatctatcatgtgctatga; NM 131576; NM_008656.
(SEQ ID NO: 67)
Myf6: NM_002469: atgatgatggacctttttgaaactggctcctatttcttctacttggatggggaaaatgttactctgca
gccattagaagtggcagaaggctctcctttgtatccagggagtgatggtaccttgtccccctgccaggaccaaatgcccccg
gaagcggggagcgacagcagcggagaggaacatgtcctggcgcccccgggcctgcagcctccacactgccccggcca
gtgtctgatctgggcttgcaagacctgcaagagaaaatctgcccccactgaccggcgaaaagccgccaccctgcgcgaaa
ggaggaggctaaagaaaatcaacgaggccttcgaggcactgaagcggcgaactgtggccaaccccaaccagaggctgc
ccaaggtggagattctgcggagcgccatcagctatattgagcggctgcaggacctgctgcaccggctggatcagcaggag
aagatgcaggagctgggggtggaccccttcagctacagacccaaacaagaaaatcttgagggtgcggatttcctgcgcac
ctgcagctcccagtggccaagtgtttccgatcattccagggggctcgtgataacggctaaggaaggaggagcaagtattga
ttcgtcagcctcgagtagccttcgatgcctttcttccatcgtggacagtatttcctcggaggaacgcaaactcccctgcgtgga
ggaagtggtggagaagtaa; NM_008657; NM_008657; NMJ) 13172.
(SEQ ID NO: 68)
Ifrd1: NM_001007245:
atgccgaagaacaagaagcggaacactccccaccgcggtagcagtgctggcggcggcgggtcaggagcagccgcagc
gacggcggcgacagcaggtggccagcatcgaaatgttcagccttttagtgatgaagatgcatcaattgaaacaatgagcca
ttgcagtggttatagcgatccttccagttttgctgaagatggaccagaagtccttgatgaggaaggaactcaagaagacctag
agtacaagttgaagggattaattgacctaaccctggataagagtgcgaagacaaggcaagcagctcttgaaggtattaaaaa
tgcactggcttcaaaaatgctgtatgaatttattctggaaaggagaatgactttaactgatagcattgaacgctgcctgaaaaaa
ggtaagagtgatgagcaacgtgcagctgcagcgttagcatctgttctttgtattcagctgggccctggaattgaaagtgaaga
gattttgaaaactcttggaccaatcctaaagaaaatcatttgtgatgggtcagctagtatgcaggctaggcaaacttgtgcaact
tgctttggtgtttgctgttttattgccacagatgacattactgaactatactcaactctggaatgtttggaaaatatcttcactaaatc
ctatctcaaagagaaagacactactgttatttgcagcactcctaatacagtgcttcatatcagctctcttcttgcatggacactact
gctgaccatatgcccaatcaatgaagtgaagaaaaagcttgagatgcatttccataagcttccaagcctcctctcttgtgatgat
gtaaacatgagaatagctgctggtgaatctttggcacttctctttgaattggccagaggaatagagagtgactttttttatgaaga
catggagtccttgacgcagatgcttagggccttggcaacagatggaaataaacaccgggccaaagtggacaagagaaag
cagcggtcagttttcagagatgtcctgagggcagtggaggaacgggattttccaacagaaaccattaaatttggtcctgaac
gcatgtatattgattgctgggtaaaaaaacacacctatgacacctttaaggaggttcttggatcagggatgcagtaccacttgc
agtcaaatgaattccttcgaaatgtatttgaacttggacccccagtgatgcttgatgctgcaacgcttaaaacgatgaagatttct
cgtttcgaaaggcatttatataactctgcagccttcaaagctcgaaccaaagctagaagcaaatgtcgagataagagagcag
atgttggagaattcttctag.
(SEQ ID NO: 69)
MeGA: NM_013172: atggggcggaagaaaatacaaatcacacgcataatggatgaaaggaaccgacaggtcact
tttacaaagagaaagtttggattaatgaagaaagcctatgaacttagtgtgctctgtgactgtgaaatagcactcatcattttcaa
cagctctaacaaactgtttcaatatgctagcactgatatggacaaagttcttctcaagtatacagaatataatgaacctcatgaaa
gcagaaccaactcggatattgttgaggctctgaacaagaaggaacacagagggtgcgacagcccagaccctgatacttcat
atgtgctaactccacatacagaagaaaaatataaaaaaattaatgaggaatttgataatatgatgcggaatcataaaatcgcac
ctggtctgccacctcagaacttttcaatgtctgtcacagttccagtgaccagccccaatgctttgtcctacactaacccaggga
gttcactggtgtccccatctttggcagccagctcaacgttaacagattcaagcatgctctctccacctcaaaccacattacatag
aaatgtgtctcctggagctcctcagagaccaccaagtactggcaatgcaggtgggatgttgagcactacagacctcacagtg
ccaaatggagctggaagcagtccagtggggaatggatttgtaaactcaagagcttctccaaatttgattggagctactggtgc
aaatagcttaggcaaagtcatgcctacaaagtctccccctccaccaggtggtggtaatcttggaatgaacagtaggaaacca
gatcttcgagttgtcatccccccttcaagcaagggcatgatgcctccactatcggaggaagaggaattggagttgaacaccc
aaaggatcagtagttctcaagccactcaacctcttgctaccccagtcgtgtctgtgacaaccccaagcttgcctccgcaagga
cttgtgtactcagcaatgccgactgcctacaacactgattattcactgaccagcgctgacctgtcagcccttcaaggcttcaac
tcgccaggaatgctgtcgctgggacaggtgtcggcctggcagcagcaccacctaggacaagcagccctcagctctcttgtt
gctggagggcagttatctcagggttccaatttatccattaataccaaccaaaacatcagcatcaagtccgaaccgatttcacct
cctcgggatcgtatgaccccatcgggcttccagcagcagcagcagcagcagcagcagcagcagccgccgccaccaccg
cagccccagccacaacccccgcagccccagccccgacaggaaatggggcgctcccctgtggacagtctgagcagctcta
gtagctcctatgatggcagtgatcgggaggatccacggggcgacttccattctccaattgtgcttggccgacccccaaacac
tgaggacagagaaagcccttctgtaaagcgaatgaggatggacgcgtgggtgacctaa.
(SEQ ID NO: 70)
Myogenin: NM_002479: atggagctgtatgagacatccccctacttctaccaggaaccccgcttctatgatgggga
aaactacctgcctgtccacctccagggcttcgaaccaccaggctacgagcggacggagctcaccctgagccccgaggcc
ccagggccccttgaggacaaggggctggggacccccgagcactgtccaggccagtgcctgccgtgggcgtgtaaggtgt
gtaagaggaagtcggtgtccgtggaccggcggcgggcggccacactgagggagaagcgcaggctcaagaaggtgaat
gaggccttcgaggccctgaagagaagcaccctgctcaaccccaaccagcggctgcccaaggtggagatcctgcgcagtg
ccatccagtacatcgagcgcctccaggccctgctcagctccctcaaccaggaggagcgtgacctccgctaccggggcgg
gggcgggccccagccaggggtgcccagcgaatgcagctctcacagcgcctcctgcagtccagagtggggcagtgcact
ggagttcagcgccaacccaggggatcatctgctcacggctgaccctacagatgcccacaacctgcactccctcacctccat
cgtggacagcatcacagtggaagatgtgtctgtggccttcccagatgaaaccatgcccaactga;
BC053899; BC068019; AB257560.
(SEQ ID NO: 71)
Nkx2.2: NM_002509: atgtcgctgaccaacacaaagacggggttttcggtcaaggacatcttagacctgccggaca
ccaacgatgaggagggctctgtggccgaaggtccggaggaagagaacgaggggcccgagccagccaagagggccgg
gccgctggggcagggcgccctggacgcggtgcagagcctgcccctgaagaaccccttctacgacagcagcgacaaccc
gtacacgcgctggctggccagcaccgagggccttcagtactccctgcacggtctggctgccggggcgccccctcaggact
caagctccaagtccccggagccctcggccgacgagtcaccggacaatgacaaggagaccccgggcggcggggggga
cgccggcaagaagcgaaagcggcgagtgcttttctccaaggcgcagacctacgagctggagcggcgctttcggcagcag
cggtacctgtcggcgcccgagcgcgaacacctggccagcctcatccgcctcacgcccacgcaggtcaagatctggttcca
gaaccaccgctacaagatgaagcgcgcccgggccgagaaaggtatggaggtgacgcccctgccctcgccgcgccgggt
ggccgtgcccgtcttggtcagggacggcaaaccatgtcacgcgctcaaagcccaggacctggcagccgccaccttccag
gcgggcattcccttttctgcctacagcgcgcagtcgctgcagcacatgcagtacaacgcccagtacagctcggccagcacc
ccccagtacccgacagcacaccccctggtccaggcccagcagtggacttggtga; NM 001077632; NMJ) 10919.
Notch
(SEQ ID NO: 72)
Notch1: NM_017617:
atgccgccgctcctggcgcccctgctctgcctggcgctgctgcccgcgctcgccgcacgaggcccgcgatgctcccagc
ccggtgagacctgcctgaatggcgggaagtgtgaagcggccaatggcacggaggcctgcgtctgtggcggggccttcgt
gggcccgcgatgccaggaccccaacccgtgcctcagcaccccctgcaagaacgccgggacatgccacgtggtggaccg
cagaggcgtggcagactatgcctgcagctgtgccctgggcttctctgggcccctctgcctgacacccctggacaatgcctg
cctcaccaacccctgccgcaacgggggcacctgcgacctgctcacgctgacggagtacaagtgccgctgcccgcccggc
tggtcagggaaatcgtgccagcaggctgacccgtgcgcctccaacccctgcgccaacggtggccagtgcctgcccttcga
ggcctcctacatctgccactgcccacccagcttccatggccccacctgccggcaggatgtcaacgagtgtggccagaagc
ccgggctttgccgccacggaggcacctgccacaacgaggtcggctcctaccgctgcgtctgccgcgccacccacactgg
ccccaactgcgagcggccctacgtgccctgcagcccctcgccctgccagaacgggggcacctgccgccccacgggcga
cgtcacccacgagtgtgcctgcctgccaggcttcaccggccagaactgtgaggaaaatatcgacgattgtccaggaaacaa
ctgcaagaacgggggtgcctgtgtggacggcgtgaacacctacaactgccgctgcccgccagagtggacaggtcagtac
tgtaccgaggatgtggacgagtgccagctgatgccaaatgcctgccagaacggcgggacctgccacaacacccacggtg
gctacaactgcgtgtgtgtcaacggctggactggtgaggactgcagcgagaacattgatgactgtgccagcgccgcctgcl
tccacggcgccacctgccatgaccgtgtggcctccttctactgcgagtgtccccatggccgcacaggtctgctgtgccaccl
caacgacgcatgcatcagcaacccctgtaacgagggctccaactgcgacaccaaccctgtcaatggcaaggccatctgca
cctgcccctcggggtacacgggcccggcctgcagccaggacgtggatgagtgctcgctgggtgccaacccctgcgagca
tgcgggcaagtgcatcaacacgctgggctccttcgagtgccagtgtctgcagggctacacgggcccccgatgcgagatcg
acgtcaacgagtgcgtctcgaacccgtgccagaacgacgccacctgcctggaccagattggggagttccagtgcatctgc
atgcccggctacgagggtgtgcactgcgaggtcaacacagacgagtgtgccagcagcccctgcctgcacaatggccgct
gcctggacaagatcaatgagttccagtgcgagtgccccacgggcttcactgggcatctgtgccagtacgatgtggacgagt
gtgccagcaccccctgcaagaatggtgccaagtgcctggacggacccaacacttacacctgtgtgtgcacggaagggtac
acggggacgcactgcgaggtggacatcgatgagtgcgaccccgacccctgccactacggctcctgcaaggacggcgtc
gccaccttcacctgcctctgccgcccaggctacacgggccaccaclgcgagaccaacatcaacgagtgctccagccagcc
ctgccgccacgggggcacctgccaggaccgcgacaacgcctacctctgcltctgcctgaaggggaccacaggacccaac
tgcgagatcaacctggatgactgtgccagcagcccctgcgactcgggcacctgtctggacaagatcgatggctacgagtgt
gcctgtgagccgggctacacagggagcatgtgtaacatcaacatcgatgagtgtgcgggcaacccctgccacaacgggg
gcacctgcgaggacggcatcaatggcttcacctgccgctgccccgagggctaccacgaccccacctgcctgtctgagglc
aatgagtgcaacagcaacccctgcgtccacggggcctgccgggacagcctcaacgggtacaagtgcgactgtgaccctg
ggtggagtgggaccaactgtgacatcaacaacaatgagtgtgaatccaacccttgtgtcaacggcggcacctgcaaagac
atgaccagtggctacgtgtgcacctgccgggagggcttcagcggtcccaactgccagaccaacatcaacgagtgtgcgtc
caacccatgtctgaaccagggcacgtgtattgacgacgttgccgggtacaagtgcaactgcctgctgccctacacaggtgc
cacgtgtgaggtgglgctggccccgtgtgcccccagcccctgcagaaacggcggggagtgcaggcaatccgaggactat
gagagcttctcctgtgtctgccccacgggctggcaagggcagacctgtgagglcgacatcaacgagtgcgttctgagcccg
tgccggcacggcgcatcctgccagaacacccacggcggctaccgclgccactgccaggccggctacagtgggcgcaac
tgcgagaccgacatcgacgactgccggcccaacccgtgtcacaacgggggctcctgcacagacggcatcaacacggcct
tclgcgactgcctgcccggcttccggggcactttctgtgaggaggacatcaacgagtgtgccagtgacccctgccgcaacg
gggccaactgcacggactgcgtggacagctacacgtgcacctgccccgcaggcttcagcgggatccactgtgagaacaa
cacgcctgactgcacagagagctcctgcttcaacggtggcacctgcgtggacggcatcaactcgttcacctgcctgtgtcca
cccggcttcacgggcagctactgccagcacgatgtcaatgagtgcgactcacagccctgcctgcatggcggcacctgtca
ggacggctgcggctcctacaggtgcacctgcccccagggctacactggccccaactgccagaaccttgtgcactggtgtg
actcctcgccctgcaagaacggcggcaaatgctggcagacccacacccagtaccgctgcgagtgccccagcggctggac
cggcctttactgcgacgtgcccagcgtgtcctgtgaggtggctgcgcagcgacaaggtgttgacgttgcccgcctgtgcca
gcatggagggctctgtgtggacgcgggcaacacgcaccactgccgctgccaggcgggctacacaggcagctactgtga
ggacctggtggacgagtgctcacccagcccctgccagaacggggccacctgcacggactacctgggcggctactcctgc
aagtgcgtggccggctaccacggggtgaactgctctgaggagatcgacgagtgcctctcccacccctgccagaacgggg
gcacctgcctcgacctccccaacacctacaagtgctcctgcccacggggcactcagggtgtgcactgtgagatcaacgtgg
acgactgcaatccccccgttgaccccgtgtcccggagccccaagtgctttaacaacggcacctgcgtggaccaggtgggc
ggctacagctgcacctgcccgccgggcttcgtgggtgagcgctgtgagggggatgtcaacgagtgcctgtccaatccctg
cgacgcccgtggcacccagaactgcgtgcagcgcgtcaatgacttccactgcgagtgccgtgctggtcacaccgggcgc
cgctgcgagtccgtcatcaatggctgcaaaggcaagccctgcaagaatgggggcacctgcgccgtggcctccaacaccg
cccgcgggttcatctgcaagtgccctgcgggcttcgagggcgccacgtgtgagaatgacgctcgtacctgcggcagcctg
cgctgcctcaacggcggcacatgcatctccggcccgcgcagccccacctgcctgtgcctgggccccttcacgggccccg
aatgccagttcccggccagcagcccctgcctgggcggcaacccctgctacaaccaggggacctgtgagcccacatccga
gagccccttctaccgttgcctgtgccccgccaaattcaacgggctcttgtgccacatcctggactacagcttcgggggtggg
gccgggcgcgacatccccccgccgctgatcgaggaggcgtgcgagctgcccgagtgccaggaggacgcgggcaaca
aggtclgcagcctgcagtgcaacaaccacgcgtgcggctgggacggcggtgactgctccctcaacttcaatgacccctgg
aagaactgcacgcagtctctgcagtgctggaagtacttcagtgacggccactgtgacagccagtgcaactcagccggctgc
ctcttcgacggctttgactgccagcgtgcggaaggccagtgcaaccccctgtacgaccagtactgcaaggaccacttcagc
gacgggcactgcgaccagggctgcaacagcgcggaglgcgagtgggacgggctggactgtgcggagcatgtacccga
gaggctggcggccggcacgctggtggtggtggtgctgatgccgccggagcagctgcgcaacagclccttccacttcctgc
gggagctcagccgcgtgctgcacaccaacgtggtcttcaagcgtgacgcacacggccagcagatgatcttcccctactac
ggccgcgaggaggagctgcgcaagcaccccatcaagcgtgccgccgagggctgggccgcacctgacgccctgctggg
ccaggtgaaggcctcgctgctccctggtggcagcgagggtgggcggcggcggagggagctggaccccatggacgtcc
gcggctccatcgtctacctggagattgacaaccggcagtgtgtgcaggcctcctcgcagtgcttccagagtgccaccgacg
tggccgcattcctgggagcgctcgcctcgctgggcagcctcaacatcccctacaagatcgaggccgtgcagagtgagacc
gtggagccgcccccgccggcgcagctgcacttcatgtacgtggcggcggccgcctttgtgcttctgttcttcgtgggctgcg
gggtgctgctgtcccgcaagcgccggcggcagcatggccagctctggttccctgagggcttcaaagtgtctgaggccagc
aagaagaagcggcgggagcccctcggcgaggactccgtgggcctcaagcccctgaagaacgcttcagacggtgccctc
atggacgacaaccagaatgagtggggggacgaggacctggagaccaagaagttccggttcgaggagcccgtggttctgc
ctgacctggacgaccagacagaccaccggcagtggactcagcagcacctggatgccgctgacctgcgcatgtctgccatg
gcccccacaccgccccagggtgaggttgacgccgactgcatggacgtcaatgtccgcgggcctgatggcttcaccccgct
catgatcgcctcctgcagcgggggcggcctggagacgggcaacagcgaggaagaggaggacgcgccggccgtcatct
ccgacttcatctaccagggcgccagcctgcacaaccagacagaccgcacgggcgagaccgccttgcacctggccgccc
gctactcacgctctgatgccgccaagcgcctgctggaggccagcgcagatgccaacatccaggacaacatgggccgcac
cccgctgcatgcggctgtgtctgccgacgcacaaggtgtcttccagatcctgatccggaaccgagccacagacctggatgc
ccgcatgcatgatggcacgacgccactgatcctggctgcccgcctggccgtggagggcatgctggaggacctcatcaact
cacacgccgacgtcaacgccgtagatgacctgggcaagtccgccctgcactgggccgccgccgtgaacaatgtggatgc
cgcagttgtgctcctgaagaacggggctaacaaagatatgcagaacaacagggaggagacacccctgtttctggccgccc
gggagggcagctacgagaccgccaaggtgctgctggaccactttgccaaccgggacatcacggatcatatggaccgcct
gccgcgcgacatcgcacaggagcgcatgcatcacgacatcgtgaggctgctggacgagtacaacctggtgcgcagccc
gcagctgcacggagccccgctggggggcacgcccaccctgtcgcccccgctctgctcgcccaacggctacctgggcag
cctcaagcccggcgtgcagggcaagaaggtccgcaagcccagcagcaaaggcctggcctgtggaagcaaggaggcca
aggacctcaaggcacggaggaagaagtcccaggacggcaagggctgcctgctggacagctccggcatgctctcgcccg
tggactccctggagtcaccccatggctacctgtcagacgtggcctcgccgccactgctgccctccccgttccagcagtctcc
gtccgtgcccctcaaccacctgcctgggatgcccgacacccacctgggcatcgggcacctgaacgtggcggccaagccc
gagatggcggcgctgggtgggggcggccggctggcctttgagactggcccacctcgtctctcccacctgcctgtggcctct
ggcaccagcaccgtcctgggctccagcagcggaggggccctgaatttcactgtgggcgggtccaccagtttgaatggtca
atgcgagtggctgtcccggctgcagagcggcatggtgccgaaccaatacaaccctctgcgggggagtgtggcaccaggc
cccctgagcacacaggccccctccctgcagcatggcatggtaggcccgctgcacagtagccttgctgccagcgccctgtc
ccagatgatgagctaccagggcctgcccagcacccggctggccacccagcctcacctggtgcagacccagcaggtgcag
ccacaaaacttacagatgcagcagcagaacctgcagccagcaaacatccagcagcagcaaagcctgcagccgccacca
ccaccaccacagccgcaccttggcgtgagctcagcagccagcggccacctgggccggagcttcctgagtggagagccg
agccaggcagacgtgcagccactgggccccagcagcctggcggtgcacactattctgccccaggagagccccgccctg
cccacgtcgctgccatcctcgctggtcccacccgtgaccgcagcccagttcctgacgcccccctcgcagcacagctactcc
tcgcctgtggacaacacccccagccaccagctacaggtgcctgagcaccccttcctcaccccgtcccctgagtcccctgac
cagtggtccagctcgtccccgcattccaacgtctccgactggtccgagggcgtctccagccctcccaccagcatgcagtcc
cagatcgcccgcattccggaggccttcaagtaa; AFI 59231.
NOTCH2: NM 024408; NMJ) 10928.
(SEQ ID NO: 73)
NOTCH3: NM000435: atggggccgggggcccgtggccgccgccgccgccgtcgcccgatgtcgccgccacc
gccaccgccacccgtgcgggcgctgcccctgctgctgctgctagcggggccgggggctgcagcccccccttgcctggac
ggaagcccgtgtgcaaatggaggtcgttgcacccagctgccctcccgggaggctgcctgcctgtgcccgcctggctgggt
gggtgagcggtgtcagctggaggacccctgtcactcaggcccctgtgctggccgtggtgtctgccagagttcagtggtggc
tggcaccgcccgattctcatgccggtgcccccgtggcttccgaggccctgactgctccctgccagatccctgcctcagcag
cccttgtgcccacggtgcccgctgctcagtggggcccgatggacgcttcctctgctcctgcccacctggctaccagggccg
cagctgccgaagcgacgtggatgagtgccgggtgggtgagccctgccgccatggtggcacctgcctcaacacacctggc
tccttccgctgccagtgtccagctggctacacagggccactatgtgagaaccccgcggtgccctgtgcaccctcaccatgc
cgtaacgggggcacctgcaggcagagtggcgacctcacttacgactgtgcctgtcttcctgggtttgagggtcagaattgtg
aagtgaacgtggacgactgtccaggacaccgatgtctcaatggggggacatgcgtggatggcgtcaacacctataactgc
cagtgccctcctgagtggacaggccagttctgcacggaggacgtggatgagtgtcagctgcagcccaacgcctgccacaa
tgggggtacctgcttcaacacgctgggtggccacagctgcgtgtgtgtcaatggctggacaggcgagagctgcagtcaga
atatcgatgactgtgccacagccgtgtgcttccatggggccacctgccatgaccgcgtggcttctttctactgtgcctgcccca
tgggcaagactggcctcctgtgtcacctggatgacgcctgtgtcagcaacccctgccacgaggatgctatctgtgacacaaa
tccggtgaacggccgggccatttgcacctgtcctcccggcttcacgggtggggcatgtgaccaggatgtggacgagtgctc
tatcggcgccaacccctgcgagcacttgggcaggtgcgtgaacacgcagggctccttcctgtgccagtgcggtcgtggcta
cactggacctcgctgtgagaccgatgtcaacgagtgtctgtcggggccctgccgaaaccaggccacgtgcctcgaccgca
taggccagttcacctgtatctgtatggcaggcttcacaggaacctattgcgaggtggacattgacgagtgtcagagtagccc
ctgtgtcaacggtggggtctgcaaggaccgagtcaatggcttcagctgcacctgcccctcgggcttcagcggctccacgtg
tcagctggacgtggacgaatgcgccagcacgccctgcaggaatggcgccaaatgcgtggaccagcccgatggctacga
gtgccgctgtgccgagggctttgagggcacgctgtgtgatcgcaacgtggacgactgctcccctgacccatgccaccatgg
tcgctgcgtggatggcatcgccagcttctcatgtgcctgtgctcctggctacacgggcacacgctgcgagagccaggtgga
cgaatgccgcagccagccctgccgccatggcggcaaatgcctagacctggtggacaagtacctctgccgctgcccttctg
ggaccacaggtgtgaactgcgaagtgaacattgacgactgtgccagcaacccctgcacctttggagtctgccgtgatggca
tcaaccgctacgactgtgtctgccaacctggcttcacagggcccctttgtaacgtggagatcaatgagtgtgcttccagccca
tgcggcgagggaggttcctgtgtggatggggaaaatggcttccgctgcctctgcccgcctggctccttgcccccactctgcc
tccccccgagccatccctgtgcccatgagccctgcagtcacggcatctgctatgatgcacctggcgggttccgctgtgtgtgt
gagcctggctggagtggcccccgctgcagccagagcctggcccgagacgcctgtgagtcccagccgtgcagggccggt
gggacatgcagcagcgatggaatgggtttccactgcacctgcccgcctggtgtccagggacgtcagtgtgaactcctctcc
ccctgcaccccgaacccctgtgagcatgggggccgctgcgagtctgcccctggccagctgcctgtctgctcctgccccca
gggctggcaaggcccacgatgccagcaggatgtggacgagtgtgctggccccgcaccctgtggccctcatggtatctgca
ccaacctggcagggagtttcagctgcacctgccatggagggtacactggcccttcctgcgatcaggacatcaatgactgtg
accccaacccatgcctgaacggtggctcgtgccaagacggcgtgggctccttttcctgctcctgcctccctggtttcgccgg
cccacgatgcgcccgcgatgtggatgagtgcctgagcaacccctgcggcccgggcacctgtaccgaccacgtggcctcc
ttcacctgcacctgcccgccaggctacggaggcttccactgcgaacaggacctgcccgactgcagccccagctcctgcttc
aatggcgggacctgtgtggacggcgtgaactcgttcagctgcctgtgccgtcccggctacacaggagcccactgccaaca
tgaggcagacccctgcctctcgcggccctgcctacacgggggcgtctgcagcgccgcccaccctggcttccgctgcacct
gcctcgagagcttcacgggcccgcagtgccagacgctggtggattggtgcagccgccagccttgtcaaaacgggggtcg
ctgcgtccagactggggcctattgcctttgtccccctggatggagcggacgcctctgtgacatccgaagcttgccctgcagg
gaggccgcagcccagatcggggtgcggctggagcagctgtgtcaggcgggtgggcagtgtgtggatgaagacagctcc
cactactgcgtgtgcccagagggccgtactggtagccactgtgagcaggaggtggacccctgcttggcccagccctgcca
gcatggggggacctgccgtggctatatggggggctacatgtgtgagtgtcttcctggctacaatggtgataactgtgaggac
gacgtggacgagtgtgcctcccagccctgccagcacgggggttcatgcattgacctcgtggcccgctatctctgctcctgtc
ccccaggaacgctgggggtgctctgcgagattaatgaggatgactgcggcccaggcccaccgctggactcagggccccg
gtgcctacacaatggcacctgcgtggacctggtgggtggtttccgctgcacctgtcccccaggatacactggtttgcgctgc
gaggcagacatcaatgagtgtcgctcaggtgcctgccacgcggcacacacccgggactgcctgcaggacccaggcgga
ggtttccgttgcctttgtcatgctggcttctcaggtcctcgctgtcagactgtcctgtctccctgcgagtcccagccatgccagc
atggaggccagtgccgtcctagcccgggtcctgggggtgggctgaccttcacctgtcactgtgcccagccgttctggggtc
cgcgttgcgagcgggtggcgcgctcctgccgggagctgcagtgcccggtgggcgtcccatgccagcagacgccccgcg
ggccgcgctgcgcctgccccccagggttgtcgggaccctcctgccgcagcttcccggggtcgccgccgggggccagca
acgccagctgcgcggccgccccctgtctccacgggggctcctgccgccccgcgccgctcgcgcccttcttccgctgcgct
tgcgcgcagggctggaccgggccgcgctgcgaggcgcccgccgcggcacccgaggtctcggaggagccgcggtgcc
cgcgcgccgcctgccaggccaagcgcggggaccagcgctgcgaccgcgagtgcaacagcccaggctgcggctggga
cggcggcgactgctcgctgagcgtgggcgacccctggcggcaatgcgaggcgctgcagtgctggcgcctcttcaacaac
agccgctgcgaccccgcctgcagctcgcccgcctgcctctacgacaacttcgactgccacgccggtggccgcgagcgca
cttgcaacccggtgtacgagaagtactgcgccgaccactttgccgacggccgctgcgaccagggctgcaacacggagga
gtgcggctgggatgggctggattgtgccagcgaggtgccggccctgctggcccgcggcgtgctggtgctcacagtgctgc
tgccgccagaggagctactgcgttccagcgccgactttctgcagcggctcagcgccatcctgcgcacctcgctgcgcttcc
gcctggacgcgcacggccaggccatggtcttcccttaccaccggcctagtcctggctccgaaccccgggcccgtcggga
gctggcccccgaggtgatcggctcggtagtaatgctggagattgacaaccggctctgcctgcagtcgcctgagaatgatca
ctgcttccccgatgcccagagcgccgctgactacctgggagcgttgtcagcggtggagcgcctggacttcccgtacccact
gcgggacgtgcggggggagccgctggagcctccagaacccagcgtcccgctgctgccactgctagtggcgggcgctgt
cttgctgctggtcattctcgtcctgggtgtcatggtggcccggcgcaagcgcgagcacagcaccctctggttccctgagggc
ttctcactgcacaaggacgtggcctctggtcacaagggccggcgggaacccgtgggccaggacgcgctgggcatgaag
aacatggccaagggtgagagcctgatgggggaggtggccacagactggatggacacagagtgcccagaggccaagcg
gctaaaggtagaggagccaggcatgggggctgaggaggctgtggattgccgtcagtggactcaacaccatctggttgctg
ctgacatccgcgtggcaccagccatggcactgacaccaccacagggcgacgcagatgctgatggcatggatgtcaatgtg
cgtggcccagatggcttcaccccgctaatgctggcttccttctgtgggggggctctggagccaatgccaactgaagaggat
gaggcagatgacacatcagctagcatcatctccgacctgatctgccagggggctcagcttggggcacggactgaccgtact
ggcgagactgctttgcacctggctgcccgttatgcccgtgctgatgcagccaagcggctgctggatgctggggcagacac
caatgcccaggaccactcaggccgcactcccctgcacacagctgtcacagccgatgcccagggtgtcttccagattctcatc
cgaaaccgctctacagacttggatgcccgcatggcagatggctcaacggcactgatcctggcggcccgcctggcagtaga
gggcatggtggaagagctcatcgccagccatgctgatgtcaatgctgtggatgagcttgggaaatcagccttacactgggct
gcggctgtgaacaacgtggaagccactttggccctgctcaaaaatggagccaataaggacatgcaggatagcaaggagg
agacccccctattcctggccgcccgcgagggcagctatgaggctgccaagctgctgttggaccactttgccaaccgtgaga
tcaccgaccacctggacaggctgccgcgggacgtagcccaggagagactgcaccaggacatcgtgcgcttgctggatca
acccagtgggccccgcagcccccccggtccccacggcctggggcctctgctctgtcctccaggggccttcctccctggcc
tcaaagcggcacagtcggggtccaagaagagcaggaggccccccgggaaggcggggctggggccgcaggggcccc
gggggcggggcaagaagctgacgctggcctgcccgggccccctggctgacagctcggtcacgctgtcgcccgtggact
cgctggactccccgcggcctttcggtgggccccctgcttcccctggtggcttcccccttgaggggccctatgcagctgccac
tgccactgcagtgtctctggcacagcttggtggcccaggccgggcgggtctagggcgccagccccctggaggatgtgtac
tcagcctgggcctgctgaaccctgtggctgtgcccctcgattgggcccggctgcccccacctgcccctccaggcccctcgtt
cctgctgccactggcgccgggaccccagctgctcaacccagggacccccgtctccccgcaggagcggcccccgccttac
ctggcagtcccaggacatggcgaggagtacccggcggctggggcacacagcagccccccaaaggcccgcttcctgcgg
gttcccagtgagcacccttacctgaccccatcccccgaatcccctgagcactgggccagcccctcacctccctccctctcag
actggtccgaatccacgcctagcccagccactgccactggggccatggccaccaccactggggcactgcctgcccagcc
acttcccttgtctgttcccagctcccttgctcaggcccagacccagctggggccccagccggaagttacccccaagaggca
agtgttggcctga.
(SEQ ID NO: 74)
Nurr1: NM_006186: atgccttgtgttcaggcgcagtatgggtcctcgcctcaaggagccagccccgcttctcagagc
tacagttaccactcttcgggagaatacagctccgatttcttaactccagagtttgtcaagtttagcatggacctcaccaacactg
aaatcactgccaccacttctctccccagcttcagtacctttatggacaactacagcacaggctacgacgtcaagccaccttgct
tgtaccaaatgcccctgtccggacagcagtcctccattaaggtagaagacattcagatgcacaactaccagcaacacagcc
acctgcccccccagtctgaggagatgatgccgcactccgggtcggtttactacaagccctcctcgcccccgacgcccacca
ccccgggcttccaggtgcagcacagccccatgtgggacgacccgggatctctccacaacttccaccagaactacgtggcc
actacgcacatgatcgagcagaggaaaacgccagtctcccgcctctccctcttctcctttaagcaatcgccccctggcaccc
cggtgtctagttgccagatgcgcttcgacgggcccctgcacgtccccatgaacccggagcccgccggcagccaccacgt
ggtggacgggcagaccttcgctgtgcccaaccccattcgcaagcccgcgtccatgggcttcccgggcctgcagatcggcc
acgcgtctcagctgctcgacacgcaggtgccctcaccgccgtcgcggggctccccctccaacgaggggctgtgcgctgtg
tgtggggacaacgcggcctgccaacactacggcgtgcgcacctgtgagggctgcaaaggcttctttaagcgcacagtgca
aaaaaatgcaaaatacgtgtgtttagcaaataaaaactgcccagtggacaagcgtcgccggaatcgctgtcagtactgccga
tttcagaagtgcctggctgttgggatggtcaaagaagtggttcgcacagacagtttaaaaggccggagaggtcgtttgccct
cgaaaccgaagagcccacaggagccctctcccccttcgcccccggtgagtctgatcagtgccctcgtcagggcccatgtc
gactccaacccggctatgaccagcctggactattccaggttccaggcgaaccctgactatcaaatgagtggagatgacacc
cagcatatccagcaattctatgatctcctgactggctccatggagatcatccggggctgggcagagaagatccctggcttcg
cagacctgcccaaagccgaccaagacctgctttttgaatcagctttcttagaactgtttgtccttcgattagcatacaggtccaa
cccagtggagggtaaactcatcttttgcaatggggtggtcttgcacaggttgcaatgcgttcgtggctttggggaatggattga
ttccattgttgaattctcctccaacttgcagaatatgaacatcgacatttctgccttctcctgcattgctgccctggctatggtcac
agagagacacgggctcaaggaacccaagagagtggaagaactgcaaaacaagattgtaaattgtctcaaagaccacgtga
ctttcaacaatggggggttgaaccgccccaattatttgtccaaactgttggggaagctcccagaacttcgtaccctttgcacac
aggggctacagcgcattttctacctgaaattggaagacttggtgccaccgccagcaataattgacaaacttttcctggacactt
tacctttctaa; NMO19328.
(SEQ ID NO: 75)
NOV(CCN3): NM_002514: atgcagagtgtgcagagcacgagcttttgtctccgaaagcagtgcctttgcctgac
cttcctgcttctccatctcctgggacaggtcgctgcgactcagcgctgccctccccagtgcccgggccggtgccctgcgac
gccgccgacctgcgcccccggggtgcgcgcggtgctggacggctgctcatgctgtctggtgtgtgcccgccagcgtggc
gagagctgctcagatctggagccatgcgacgagagcagtggcctctactgtgatcgcagcgcggaccccagcaaccaga
ctggcatctgcacggcggtagagggagataactgtgtgttcgatggggtcatctaccgcagtggagagaaatttcagccaa
gctgcaaattccagtgcacctgcagagatgggcagattggctgtgtgccccgctgtcagctggatgtgctactgcctgagcc
taactgcccagctccaagaaaagttgaggtgcctggagagtgctgtgaaaagtggatctgtggcccagatgaggaggattc
actgggaggccttacccttgcagcttacaggccagaagccaccctaggagtagaagtctctgactcaagtgtcaactgcatt
gaacagaccacagagtggacagcatgctccaagagctgtggtatggggttctccacccgggtcaccaataggaaccgtca
atgtgagatgctgaaacagactcggctctgcatggtgcggccctgtgaacaagagccagagcagccaacagataagaaa
ggaaaaaagtgtctccgcaccaagaagtcactcaaagccatccacctgcagttcaagaactgcaccagcctgcacacctac
aagcccaggttctgtggggtctgcagtgatggccgctgctgcactccccacaataccaaaaccatccaggcagagtttcagt
gctccccagggcaaatagtcaagaagccagtgatggtcattgggacctgcacctgtcacaccaactgtcctaagaacaatg
aggccttcctccaggagctggagctgaagactaccagagggaaaatgtaa; NM_010930; NM030868; BCO15028.
(SEQ ID NO: 76)
OLIG1:NM_138983: atgctgcggccacagcggcccggagacttgcagctcggggcctccctctacgagctggtg
ggctacaggcagccgccctcctcctcctcctcctccacctcctccacctcctccacttcctcctcctccacgacggcccccct
cctccccaaggctgcgcgcgagaagccggaggcgccggccgagcctccaggccccgggcccgggtcaggcgcgcac
ccgggcggcagcgcccggccggacgccaaggaggagcagcagcagcagctgcggcgcaagatcaacagccgcgag
cggaagcgcatgcaggacctgaacctggccatggacgccctgcgcgaggtcatcctgccctactcagcggcgcactgcc
agggcgcgcccggccgcaagctctccaagatagccacgctgctgctcgcccgcaactacatcctactgctgggcagctcg
ctgcaggagctgcgccgcgcgctgggcgagggcgccgggcccgccgcgccgcgcctgctgctggccgggctgcccct
gctcgccgccgcgcccggctccgtgttgctggcgcccggcgccgtaggaccccccgacgcgctgcgccccgccaagta
cctgtcgctggcgctggacgagccgccgtgcggccagttcgctctccccggcggcggcgcaggcggccccggcctctg
cacctgcgccgtgtgcaagttcccgcacctggtcccggccagcctgggcctggccgccgtgcaggcgcaattctccaagt
ga; NM016968; NMJ)01020796.
(SEQ ID NO: 77)
OLIGiNM\hat{ }OSδOóiatggactcggacgccagcctggtgtccagccgcccgtcgtcgccagagcccgatgaccttt
ttctgccggcccggagtaagggcagcagcggcagcgccttcactgggggcaccgtgtcctcgtccaccccgagtgactg
cccgccggagctgagcgccgagctgcgcggcgctatgggctctgcgggcgcgcatcctggggacaagctaggaggca
gtggcttcaagtcatcctcgtccagcacctcgtcgtctacgtcgtcggcggctgcgtcgtccaccaagaaggacaagaagc
aaatgacagagccggagctgcagcagctgcgtctcaagatcaacagccgcgagcgcaagcgcatgcacgacctcaacat
cgccatggatggcctccgcgaggtcatgccgtacgcacacggcccttcggtgcgcaagctttccaagatcgccacgctgct
gctggcgcgcaactacatcctcatgctcaccaactcgctggaggagatgaagcgactggtgagcgagatctacgggggcc
accacgctggcttccacccgtcggcctgcggcggcctggcgcactccgcgcccctgcccgccgccaccgcgcacccgg
cagcagcagcgcacgccgcacatcaccccgcggtgcaccaccccatcctgccgcccgccgccgcagcggctgctgccg
ccgctgcagccgcggctgtgtccagcgcctctctgcccggatccgggctgccgtcggtcggctccatccgtccaccgcac
ggcctactcaagtctccgtctgctgccgcggccgccccgctggggggcgggggcggcggcagtggggcgagcggggg
cttccagcactggggcggcatgccctgcccctgcagcatgtgccaggtgccgccgccgcaccaccacgtgtcggctatgg
gcgccggcagcctgccgcgcctcacctccgacgccaagtga.
(SEQ ID NO: 78)
PcIxI: NM000209: atgaacggcgaggagcagtactacgcggccacgcagctttacaaggacccatgcgcgttcca
gcgaggcccggcgccggagttcagcgccagcccccctgcgtgcctgtacatgggccgccagcccccgccgccgccgc
cgcacccgttccctggcgccctgggcgcgctggagcagggcagccccccggacatctccccgtacgaggtgccccccct
cgccgacgaccccgcggtggcgcaccttcaccaccacctcccggctcagctcgcgctcccccacccgcccgccgggcc
cttcccggagggagccgagccgggcgtcctggaggagcccaaccgcgtccagctgcctttcccatggatgaagtctacca
aagctcacgcgtggaaaggccagtgggcaggcggcgcctacgctgcggagccggaggagaacaagcggacgcgcac
ggcctacacgcgcgcacagctgctagagctggagaaggagttcctattcaacaagtacatctcacggccgcgccgggtgg
agctggctgtcatgttgaacttgaccgagagacacatcaagatctggttccaaaaccgccgcatgaagtggaaaaaggagg
aggacaagaagcgcggcggcgggacagctgtcgggggtggcggggtcgcggagcctgagcaggactgcgccgtgac
ctccggcgaggagcttctggcgctgccgccgccgccgccccccggaggtgctgtgccgcccgctgcccccgttgccgcc
cgagagggccgcctgccgcctggccttagcgcgtcgccacagccctccagcgtcgcgcctcggcggccgcaggaacca
cgatga; NM008814; NM022852.
(SEQ ID NO: 79)
Pet1(FEV):BC138435; NM017521: atgagacagagcggcgcctcccagcccctgctgatcaacatgtacctg
ccagatcccgtcggagacggtctcttcaaggacgggaagaacccgagctgggggccgctgagccccgcggttcagaaa
ggcagcggacagatccagctgtggcagtttctgctggagctgctggctgaccgcgcgaacgccggctgcatcgcgtggg
agggcggtcacggcgagttcaagctcacggacccggacgaggtggcgcggcggtggggcgagcgcaagagcaagcc
caacatgaactacgacaagctgagccgcgccctgcgctactactacgacaagaacatcatgagcaaggtgcatggcaagc
gctacgcctaccgcttcgacttccagggcctggcgcaggcctgccagccgccgcccgcgcacgctcatgccgccgccgc
agctgctgccgccgccgcggccgcccaggacggcgcgctctacaagctgcccgccggcctcgccccgctgcccttcccc
ggcctctccaaactcaacctcatggccgcctcggccggggtcgcgcccgccggcttctcctactggccgggcccgggcc
ccgccgccaccgctgccgccgccaccgccgcgctctaccccagtcccagcttgcagcccccgcccgggcccttcgggg
ccgtggccgcagcctcgcacttggggggccattaccactag; NG_002690; NP059991.
(SEQ ID NO: 80)
Phox2a: NM_005169: atggactactcctacctcaattcgtacgactcgtgcgtggcggccatggaggcgtccgccta
cggcgactttggcgcctgcagccagcccggcggcttccaatacagccccctgcggcccgctttccccgcggcagggccg
ccctgccccgcgctcggctcctccaactgcgcacttggcgccctacgcgaccaccagcccgcgccctactcggcagtgcc
ctacaagttcttcccagagccatccggcctgcacgagaagcgcaagcagcggcgcatccgcaccacgttcaccagcgcg
cagctcaaggagctggagcgcgttttcgctgagacccactaccccgacatttacacgcgtgaggagctggcgctcaagatc
gacctcactgaggctcgcgtgcaggtctggttccagaaccgccgggccaagttccgcaaacaggagcgcgcggccagc
gccaagggcgcggcgggcgcggcgggcgccaaaaagggcgaggcgcgctgctcctccgaggacgacgattccaagg
agtccacgtgcagccccacgcccgatagcaccgcctcgctgccgccgccgcctgcgcccggcctggccagcccgcgcc
tgagccccagcccgctgcccgtcgcactgggctccgggccgggacctgggccggggccacagccgctcaagggcgca
ctgtgggccggtgtggcgggcggtgggggcggcgggcctggcgcgggagcggccgaactacttaaggcttggcagcc
ggcggagtccggccccgggcccttctccggggttctgtcctcctttcaccggaagcccggccccgccctgaagaccaatct
cttctag; AJ320270; AY371497; AY371496.
(SEQ ID NO: 81)
Phox2b: NM_003924: atgtataaaatggaatattcttacctcaattcctctgcctacgagtcctgtatggctgggatgg
acacctcgagcctggcttcagcctatgctgacttcagttcctgcagccaggccagtggcttccagtataacccgataaggac
cacttttggggccacgtccggctgcccttccctcacgccgggatcctgcagcctgggcaccctcagggaccaccagagca
gtccgtacgccgcagttccttacaaactcttcacggaccacggcggcctcaacgagaagcgcaagcagcggcgcatccgc
accactttcaccagtgcccagctcaaagagctggaaagggtcttcgcggagactcactaccccgacatctacactcgggag
gagctggccctgaagatcgacctcacagaggcgcgagtccaggtgtggttccagaaccgccgcgccaagtttcgcaagc
aggagcgcgcagcggcagccgcagcggccgcggccaagaacggctcctcgggcaaaaagtctgactcttccagggac
gacgagagcaaagaggccaagagcactgacccggacagcactgggggcccaggtcccaatcccaaccccacccccag
ctgcggggcgaatggaggcggcggcggcgggcccagcccggctggagctccgggggcggcggggcccgggggcc
cgggaggcgaacccggcaagggcggcgcagcagcagcggcggcggccgcggcagcggcggcggcggcagcggc
agcggcggcagctggaggcctggctgcggctgggggccctggacaaggctgggctcccggccccggccccatcacct
ccatcccggattcgcttgggggtcccttcgccagcgtcctatcttcgctccaaagacccaacggtgccaaagccgccttagt
gaagagcagtatgttctga; NM008888; AY371498; Y14493.
(SEQ ID NO: 82)
Pit1: NM_000306: atgagttgccaagcttttacttcggctgatacctttatacctctgaattctgacgcctctgcaactctg
cctctgataatgcatcacagtgctgccgagtgtctaccagtctccaaccatgccaccaatgtgatgtctacagcaacaggactt
cattattctgttccttcctgtcattatggaaaccagccatcaacctatggagtgatggcaggtagtttaaccccttgtctttataaat
ttcctgaccacaccttgagtcatggatttcctcctatacaccagcctcttctggcagaggaccccacagctgctgatttcaagc
aggaactcaggcggaaaagtaaattggtggaagagccaatagacatggattctccagaaatcagagaacttgaaaagtttg
ccaatgaatttaaagtgagacgaattaaattaggatacacccagacaaatgttggggaggccctggcagctgtgcatggctc
tgaattcagtcaaacaacaatctgccgatttgaaaatctgcagctcagctttaaaaatgcatgcaaactgaaagcaatattatcc
aaatggctggaggaagctgagcaagtaggagctttgtacaatgaaaaagtgggagcaaatgaaaggaaaagaaaacgaa
gaacaactataagcattgctgctaaagatgctctggagagacactttggagaacagaataaaccttcttctcaagagatcatg
aggatggctgaagaactgaatctggagaaagaagtagtaagagtttggttttgcaaccggaggcagagagaaaaacgggt
gaaaacaagtctgaatcagagtttattttctatttctaaggaacatcttgagtgcagataa; M23253.
(SEQ ID NO: 83)
PITX3: NM_005029: atggagttcggcctgctcagcgaggcagaggcccggagccctgccctgtcgctgtcagac
gctggcactccgcacccccagctcccagagcacggctgcaagggccaggagcacagcgactcagaaaaggcctcggct
tcgctgcccggcggctccccagaggacggttcgctgaaaaagaagcagcggcggcagcgcacgcacttcaccagccag
cagctacaggagctagaggcgaccttccagaggaaccgctaccccgacatgagcacgcgcgaggagatcgccgtgtgg
accaacctcaccgaggcccgcgtgcgggtgtggttcaagaaccggcgcgccaaatggcggaagcgcgagcgcagcca
gcaggccgagctatgcaaaggcagcttcgcggcgccgctcggggggctggtgccgccctacgaggaggtgtaccccgg
ctactcgtacggcaactggccgcccaaggctcttgccccgccgctcgccgccaagacctttccattcgccttcaactcggtc
aacgtggggcctctggcttcgcagcccgtcttctcgccacccagctccatcgccgcctccatggtgccctccgccgcggct
gccccgggcaccgtgccagggcctggggccctgcagggcctgggcgggggcccccccgggctggctccggccgccg
tgtcctccggggccgtgtcctgcccttatgcctcggccgccgccgccgccgcggctgccgcctcttccccctacgtctatcg
ggacccgtgtaactcgagcctggccagcctgcggctcaaagccaaacagcacgcctccttcagctaccccgctgtgcacg
ggccgcccccggcagccaaccttagtccgtgccagtacgccgtggaaaggcccgtatga; NM008852; NM008987;
(SEQ ID NO: 84)
RUNX1: NM001001890: atgcgtatccccgtagatgccagcacgagccgccgcttcacgccgccttccaccgcg
ctgagcccaggcaagatgagcgaggcgttgccgctgggcgccccggacgccggcgctgccctggccggcaagctgag
gagcggcgaccgcagcatggtggaggtgctggccgaccacccgggcgagctggtgcgcaccgacagccccaacttcct
ctgctccgtgctgcctacgcactggcgctgcaacaagaccctgcccatcgctttcaaggtggtggccctaggggatgttcca
gatggcactctggtcactgtgatggctggcaatgatgaaaactactcggctgagctgagaaatgctaccgcagccatgaag
aaccaggttgcaagatttaatgacctcaggtttgtcggtcgaagtggaagagggaaaagcttcactctgaccatcactgtcttc
acaaacccaccgcaagtcgccacctaccacagagccatcaaaatcacagtggatgggccccgagaacctcgaagacatc
ggcagaaactagatgatcagaccaagcccgggagcttgtccttttccgagcggctcagtgaactggagcagctgcggcgc
acagccatgagggtcagcccacaccacccagcccccacgcccaaccctcgtgcctccctgaaccactccactgcctttaac
cctcagcctcagagtcagatgcaggatacaaggcagatccaaccatccccaccgtggtcctacgatcagtcctaccaatac
ctgggatccattgcctctccttctgtgcacccagcaacgcccatttcacctggacgtgccagcggcatgacaaccctctctgc
agaactttccagtcgactctcaacggcacccgacctgacagcgttcagcgacccgcgccagttccccgcgctgccctccat
ctccgacccccgcatgcactatccaggcgccttcacctactccccgacgccggtcacctcgggcatcggcatcggcatgtc
ggccatgggctcggccacgcgctaccacacctacctgccgccgccctaccccggctcgtcgcaagcgcagggaggccc
gttccaagccagctcgccctcctaccacctgtactacggcgcctcggccggctcctaccagttctccatggtgggcggcga
gcgctcgccgccgcgcatcctgccgccctgcaccaacgcctccaccggctccgcgctgctcaaccccagcctcccgaac
cagagcgacgtggtggaggccgagggcagccacagcaactcccccaccaacatggcgccctccgcgcgcctggagga
ggccgtgtggaggccctactga; AY509916; AY509915; NM_001001890.2; NP001001890.1;
NM001122607.1; NP001116079.1; NM001754.4; NPJ)01745.2
(SEQ ID NO: 85)
Runx2: NMJ)01015051: atgcttcattcgcctcacaaacaaccacagaaccacaagtgcggtgcaaactttctccag
gaggacagcaagaagtctctggtttttaaatggttaatctccgcaggtcactaccagccaccgagaccaacagagtcatttaa
ggctgcaagcagtatttacaacagagggtacaagttctatctgaaaaaaaaaggagggactatggcatcaaacagcctcttc
agcacagtgacaccatgtcagcaaaacttcttttgggatccgagcaccagccggcgcttcagccccccctccagcagcctg
cagcccggcaaaatgagcgacgtgagcccggtggtggctgcgcaacagcagcagcaacagcagcagcagcaacagca
gcagcagcagcagcaacagcagcagcagcagcaggaggcggcggcggcggctgcggcggcggcggcggctgcgg
cggcggcagctgcagtgccccggttgcggccgccccacgacaaccgcaccatggtggagatcatcgccgaccacccgg
ccgaactcgtccgcaccgacagccccaacttcctgtgctcggtgctgccctcgcactggcgctgcaacaagaccctgcccg
tggccttcaaggtggtagccctcggagaggtaccagatgggactgtggttactgtcatggcgggtaacgatgaaaattattct
gctgagctccggaatgcctctgctgttatgaaaaaccaagtagcaaggttcaacgatctgagatttgtgggccggagtggac
gaggcaagagtttcaccttgaccataaccgtcttcacaaatcctccccaagtagctacctatcacagagcaattaaagttacag
tagatggacctcgggaacccagaaggcacagacagaagcttgatgactctaaacctagtttgttctctgaccgcctcagtgat
ttagggcgcattcctcatcccagtatgagagtaggtgtcccgcctcagaacccacggccctccctgaactctgcaccaagtc
cttttaatccacaaggacagagtcagattacagaccccaggcaggcacagtcttccccgccgtggtcctatgaccagtcttac
ccctcctacctgagccagatgacgtccccgtccatccactctaccaccccgctgtcttccacacggggcactgggcttcctg
ccatcaccgatgtgcctaggcgcatttcaggtgcttcagaactgggccctttttcagaccccaggcagttcccaagcatttcat
ccctcactgagagccgcttctccaacccacgaatgcactatccagccacctttacttacaccccgccagtcacctcaggcatg
tccctcggtatgtccgccaccactcactaccacacctacctgccaccaccctaccccggctcttcccaaagccagagtggac
ccttccagaccagcagcactccatatctctactatggcacttcgtcaggatcctatcagtttcccatggtgccggggggagac
cggtctccttccagaatgcttccgccatgcaccaccacctcgaatggcagcacgctattaaatccaaatttgcctaaccagaat
gatggtgttgacgctgatggaagccacagcagttccccaactgttttgaattctagtggcagaatggatgaatctgtttggcga
ccatattga; NM001015051.2; NP001015051.2; NM001015051; NM001024630.2;
NP001019801.2; NM004348.3; NP004339.3.
(SEQ ID NO: 86)
Shh: NM_000193: atgctgctgctggcgagatgtctgctgctagtcctcgtctcctcgctgctggtatgctcgggactg
gcgtgcggaccgggcagggggttcgggaagaggaggcaccccaaaaagctgacccctttagcctacaagcagtttatcc
ccaatgtggccgagaagaccctaggcgccagcggaaggtatgaagggaagatctccagaaactccgagcgatttaagga
actcacccccaattacaaccccgacatcatatttaaggatgaagaaaacaccggagcggacaggctgatgactcagaggtg
taaggacaagttgaacgctttggccatctcggtgatgaaccagtggccaggagtgaaactgcgggtgaccgagggctggg
acgaagatggccaccactcagaggagtctctgcactacgagggccgcgcagtggacatcaccacgtctgaccgcgaccg
cagcaagtacggcatgctggcccgcctggcggtggaggccggcttcgactgggtgtactacgagtccaaggcacatatcc
actgctcggtgaaagcagagaactcggtggcggccaaatcgggaggctgcttcccgggctcggccacggtgcacctgga
gcagggcggcaccaagctggtgaaggacctgagccccggggaccgcgtgctggcggcggacgaccagggccggctg
ctctacagcgacttcctcactttcctggaccgcgacgacggcgccaagaaggtcttctacgtgatcgagacgcgggagccg
cgcgagcgcctgctgctcaccgccgcgcacctgctctttgtggcgccgcacaacgactcggccaccggggagcccgag
gcgtcctcgggctcggggccgccttccgggggcgcactggggcctcgggcgctgttcgccagccgcgtgcgcccgggc
cagcgcgtgtacgtggtggccgagcgtgacggggaccgccggctcctgcccgccgctgtgcacagcgtgaccctaagc
gaggaggccgcgggcgcctacgcgccgctcacggcccagggcaccattctcatcaaccgggtgctggcctcgtgctacg
cggtcatcgaggagcacagctgggcgcaccgggccttcgcgcccttccgcctggcgcacgcgctcctggctgcactggc
gcccgcgcgcacggaccgcggcggggacagcggcggcggggaccgcgggggcggcggcggcagagtagccctaa
ccgctccaggtgctgccgacgctccgggtgcgggggccaccgcgggcatccactggtactcgcagctgctctaccaaata
ggcacctggctcctggacagcgaggccctgcacccgctgggcatggcggtcaagtccagctga;
NP000184; NM_009170; NP033196; NM20482]; NP_990152.
(SEQ ID NO: 87)
Sox9: NM_000346: atgaatctcctggaccccttcatgaagatgaccgacgagcaggagaagggcctgtccggcgc
ccccagccccaccatgtccgaggactccgcgggctcgccctgcccgtcgggctccggctcggacaccgagaacacgcg
gccccaggagaacacgttccccaagggcgagcccgatctgaagaaggagagcgaggaggacaagttccccgtgtgcat
ccgcgaggcggtcagccaggtgctcaaaggctacgactggacgctggtgcccatgccggtgcgcgtcaacggctccagc
aagaacaagccgcacgtcaagcggcccatgaacgccttcatggtgtgggcgcaggcggcgcgcaggaagctcgcgga
ccagtacccgcacttgcacaacgccgagctcagcaagacgctgggcaagctctggagacttctgaacgagagcgagaag
cggcccttcgtggaggaggcggagcggctgcgcgtgcagcacaagaaggaccacccggattacaagtaccagccgcg
gcggaggaagtcggtgaagaacgggcaggcggaggcagaggaggccacggagcagacgcacatctcccccaacgcc
atcttcaaggcgctgcaggccgactcgccacactcctcctccggcatgagcgaggtgcactcccccggcgagcactcggg
gcaatcccagggcccaccgaccccacccaccacccccaaaaccgacgtgcagccgggcaaggctgacctgaagcgag
aggggcgccccttgccagaggggggcagacagccccctatcgacttccgcgacgtggacatcggcgagctgagcagcg
acgtcatctccaacatcgagaccttcgatgtcaacgagtttgaccagtacctgccgcccaacggccacccgggggtgccgg
ccacgcacggccaggtcacctacacgggcagctacggcatcagcagcaccgcggccaccccggcgagcgcgggccac
gtgtggatgtccaagcagcaggcgccgccgccacccccgcagcagcccccacaggccccgccggccccgcaggcgcc
cccgcagccgcaggcggcgcccccacagcagccggcggcacccccgcagcagccacaggcgcacacgctgaccacg
ctgagcagcgagccgggccagtcccagcgaacgcacatcaagacggagcagctgagccccagccactacagcgagca
gcagcagcactcgccccaacagatcgcctacagccccttcaacctcccacactacagcccctcctacccgcccatcacccg
ctcacagtacgactacaccgaccaccagaactccagctcctactacagccacgcggcaggccagggcaccggcctctact
ccaccttcacctacatgaaccccgctcagcgccccatgtacacccccatcgccgacacctctggggtcccttccatcccgca
gacccacagcccccagcactgggaacaacccgtctacacacagctcactcgaccttga
NM000346; NP000337; NM011448; NP035578; XM_343981; XP_343982.
(SEQ ID NO: 88)
sox17: NM_022454: atgagcagcccggatgcgggatacgccagtgacgaccagagccagacccagagcgcgct
gcccgcggtgatggccgggctgggcccctgcccctgggccgagtcgctgagccccatcggggacatgaaggtgaaggg
cgaggcgccggcgaacagcggagcaccggccggggccgcgggccgagccaagggcgagtcccgtatccggcggcc
gatgaacgctttcatggtgtgggctaaggacgagcgcaagcggctggcgcagcagaatccagacctgcacaacgccgag
ttgagcaagatgctgggcaagtcgtggaaggcgctgacgctggcggagaagcggcccttcgtggaggaggcagagcgg
ctgcgcgtgcagcacatgcaggaccaccccaactacaagtaccggccgcggcggcgcaagcaggtgaagcggctgaa
gcgggtggagggcggcttcctgcacggcctggctgagccgcaggcggccgcgctgggccccgagggcggccgcgtg
gccatggacggcctgggcctccagttccccgagcagggcttccccgccggcccgccgctgctgcctccgcacatgggcg
gccactaccgcgactgccagagtctgggcgcgcctccgctcgacggctacccgttgcccacgcccgacacgtccccgct
ggacggcgtggaccccgacccggctttcttcgccgccccgatgcccggggactgcccggcggccggcacctacagcta
cgcgcaggtctcggactacgctggccccccggagcctcccgccggtcccatgcacccccgactcggcccagagcccgc
gggtccctcgattccgggcctcctggcgccacccagcgcccttcacgtgtactacggcgcgatgggctcgcccggggcg
ggcggcgggcgcggcttccagatgcagccgcaacaccagcaccagcaccagcaccagcaccaccccccgggccccg
gacagccgtcgccccctccggaggcactgccctgccgggacggcacggaccccagtcagcccgccgagctcctcggg
gaggtggaccgcacggaatttgaacagtatctgcacttcgtgtgcaagcctgagatgggcctcccctaccaggggcatgac
tccggtgtgaatctccccgacagccacggggccatttcctcggtggtgtccgacgccagctccgcggtatattactgcaact
atcctgacgtgtga; BC140307; NM_011441.
(SEQ ID NO: 89)
DLX2: NM004405: atgactggagtctttgacagtctagtggctgatatgcactcgacccagatcgccgcctccagca
cgtaccaccagcaccagcagcccccgagcggcggcggcgccggcccgggtggcaacagcagcagcagcagcagcct
ccacaagccccaggagtcgcccacccttccggtgtccaccgccaccgacagcagctactacaccaaccagcagcacccg
gcgggcggcggcggcggcgggggctcgccctacgcgcacatgggttcctaccagtaccaagccagcggcctcaacaa
cgtcccttactccgccaagagcagctatgacctgggctacaccgccgcctacacctcctacgctccctatggaaccagttcg
tccccagccaacaacgagcctgagaaggaggaccttgagcctgaaattcggatagtgaacgggaagccaaagaaagtcc
ggaaaccccgcaccatctactccagtttccagctggcggctcttcagcggcgtttccaaaagactcaatacttggccttgccg
gagcgagccgagctggcggcctctctgggcctcacccagactcaggtcaaaatctggttccagaaccgccggtccaagtt
caagaagatgtggaaaagtggtgagatcccctcggagcagcaccctggggccagcgcttctccaccttgtgcttcgccgcc
agtctcagcgccggcctcctgggactttggtgtgccgcagcggatggcgggcggcggtggtccgggcagtggcggcag
cggcgccggcagctcgggctccagcccgagcagcgcggcctcggcttttctgggcaactacccctggtaccaccagacc
tcgggatccgcctcacacctgcaggccacggcgccgctgctgcaccccactcagaccccgcagccgcatcaccaccacc
accatcacggcggcgggggcgccccggtgagcgcggggacgattttctaa; NP_004396.1; NM010054.
(SEQ ID NO: 90)
DLX5: NM_005221: atgacaggagtgtttgacagaagggtccccagcatccgatccggcgacttccaagctccgtt
ccagacgtccgcagctatgcaccatccgtctcaggaatcgccaactttgcccgagtcttcagctaccgattctgactactaca
gccctacggggggagccccgcacggctactgctctcctacctcggcttcctatggcaaagctctcaacccctaccagtatca
gtatcacggcgtgaacggctccgccgggagctacccagccaaagcttatgccgactatagctacgctagctcctaccacca
gtacggcggcgcctacaaccgcgtcccaagcgccaccaaccagccagagaaagaagtgaccgagcccgaggtgagaa
tggtgaatggcaaaccaaagaaagttcgtaaacccaggactatttattccagctttcagctggccgcattacagagaaggttt
cagaagactcagtacctcgccttgccggaacgcgccgagctggccgcctcgctgggattgacacaaacacaggtgaaaat
ctggtttcagaacaaaagatccaagatcaagaagatcatgaaaaacggggagatgcccccggagcacagtcccagctcca
gcgacccaatggcgtgtaactcgccgcagtctccagcggtgtgggagccccagggctcgtcccgctcgctcagccacca
ccctcatgcccaccctccgacctccaaccagtccccagcgtccagctacctggagaactctgcatcctggtacacaagtgca
gccagctcaatcaattcccacctgccgccgccgggctccttacagcacccgctggcgctggcctccgggacactctattag;;
NM005221; NP005212.
(SEQ ID NO: 91)
HES1: NM_005524: atgccagctgatataatggagaaaaattcctcgtccccggtggctgctaccccagccagtgtca
acacgacaccggataaaccaaagacagcatctgagcacagaaagtcatcaaagcctattatggagaaaagacgaagagc
aagaataaatgaaagtctgagccagctgaaaacactgattttggatgctctgaagaaagatagctcgcggcattccaagctg
gagaaggcggacattctggaaatgacagtgaagcacctccggaacctgcagcgggcgcagatgacggctgcgctgagc
acagacccaagtgtgctggggaagtaccgagccggcttcagcgagtgcatgaacgaggtgacccgcttcctgtccacgtg
cgagggcgttaataccgaggtgcgcactcggctgctcggccacctggccaactgcatgacccagatcaatgccatgacct
accccgggcagccgcaccccgccttgcaggcgccgccaccgcccccaccgggacccggcggcccccagcacgcgcc
gttcgcgccgccgccgccactcgtgcccatccccgggggcgcggcgccccctcccggcggcgccccctgcaagctggg
cagccaggctggagaggcggctaaggtgtttggaggcttccaggtggtaccggctcccgatggccagtttgctttcctcatt
cccaacggggccttcgcgcacagcggccctgtcatccccgtctacaccagcaacagcggcacctccgtgggccccaacg
cagtgtcaccttccagcggcccctcgcttacggcggactccatgtggaggccgtggcggaactga;
NP005515.1; NM008235; NP032261.
(SEQ ID NO: 92)
FGF8: NM_006119: atgggcagcccccgctccgcgctgagctgcctgctgttgcacttgctggtcctctgcctccaa
gcccaggtaactgttcagtcctcacctaattttacacagcatgtgagggagcagagcctggtgacggatcagctcagccgcc
gcctcatccggacctaccaactctacagccgcaccagcgggaagcacgtgcaggtcctggccaacaagcgcatcaacgc
catggcagaggacggcgaccccttcgcaaagctcatcgtggagacggacacctttggaagcagagttcgagtccgagga
gccgagacgggcctctacatctgcatgaacaagaaggggaagctgatcgccaagagcaacggcaaaggcaaggactgc
gtcttcacggagattgtgctggagaacaactacacagcgctgcagaatgccaagtacgagggctggtacatggccttcacc
cgcaagggccggccccgcaagggctccaagacgcggcagcaccagcgtgaggtccacttcatgaagcggctgccccg
gggccaccacaccaccgagcagagcctgcgcttcgagttcctcaactacccgcccttcacgcgcagcctgcgcggcagc
cagaggacttgggcccccgagccccgatag; NM010205; NP034335; NM010205; NP034335; NP006110
NM033163; NPJ49353; NM033164; NP149354; NM033165; NPJ49355.
(SEQ ID NO: 93)
PITX2: NM_000325: atgaactgcatgaaaggcccgcttcacttggagcaccgagcagcggggaccaagctgtcg
gccgtctcctcatcttcctgtcaccatccccagccgttagccatggcttcggttctggctcccggtcagccccggtcgctgga
ctcctccaagcacaggctggaggtgcacaccatctccgacacctccagcccggaggccgcagagaaagataaaagccag
caggggaagaatgaggacgtgggcgccgaggacccgtctaagaagaagcggcaaaggcggcagcggactcactttac
cagccagcagctccaggagctggaggccactttccagaggaaccgctacccggacatgtccacacgcgaagaaatcgct
gtgtggaccaaccttacggaagcccgagtccgggtttggttcaagaatcgtcgggccaaatggagaaagagggagcgca
accagcaggccgagctatgcaagaatggcttcgggccgcagttcaatgggctcatgcagccctacgacgacatgtaccca
ggctattcctacaacaactgggccgccaagggccttacatccgcctccctatccaccaagagcttccccttcttcaactctatg
aacgtcaaccccctgtcatcacagagcatgttttccccacccaactctatctcgtccatgagcatgtcgtccagcatggtgccc
tcagcagtgacaggcgtcccgggctccagtctcaacagcctgaataacttgaacaacctgagtagcccgtcgctgaattcc
gcggtgccgacgcctgcctgtccttacgcgccgccgactcctccgtatgtttatagggacacgtgtaactcgagcctggcca
gcctgagactgaaagcaaagcagcactccagcttcggctacgccagcgtgcagaacccggcctccaacctgagtgcttgc
cagtatgcagtggaccggcccgtgtga; NM000325; NP000316; NM153426; NP700475; NMJ53427;
NP700476; NM001042502; NP001035967; NM001042504; NP001035969.
(SEQ ID NO: 94)
REST4: DQ644039: atggccacccaggtgatggggcagtcttctggaggaggcagtctcttcaacaacagtgccaa
catgggcatggccttaaccaacgacatgtacgacctgcacgagctctcgaaagctgaactggcagcccctcagctcatcat
gttagccaacgtggccctgacgggggaggcaagcggcagctgctgcgattacctggtcggtgaagagaggcagatggc
cgaattgatgcccgtgggagacaaccacttctcagaaagtgaaggagaaggcctggaagagtcggctgacctcaaaggg
ctggaaaacatggaactgggaagtttggagctaagtgctgtagaaccccagcccgtatttgaagcctcagctgccccagaa
atatacagcgccaataaagatcccgctccagaaacacccgtggcggaagacaaatgcaggagttctaaggccaagccctt
ccggtgtaagccttgccagtacgaagccgaatctgaagagcagtttgtgcatcacatccggattcacagcgctaagaagttc
tttgtggaggaaagtgcagagaaacaggccaaagcctgggagtcggggtcgtctccggccgaagagggcgagttctcca
aaggccccatccgctgtgaccgctgtggctacaataccaaccggtatgaccactacatggcacacctgaagcaccacctgc
gagctggcgagaacgagcgcatctacaagtgcatcatctgcacgtacacgacggtcagcgagtaccactggaggaaaca
cctgagaaaccatttccccaggaaagtctacacctgcagcaagtgcaactacttctcagacagaaaaaataactacgttcag
cacgtgcgaactcacacaggagaacgcccgtataaatgtgaactttgtccttactcaagctctcagaagactcatctaacgcg
acacatgcggactcattcagagtgtgatctagctgggtga.
(SEQ ID NO: 95)
CREBbindingprotein: NM_134442: atgaccatggaatctggagccgagaaccagcagagtggagatgcag
ctgtaacagaagctgaaaaccaacaaatgacagttcaagcccagccacagattgccacattagcccaggtatctatgccag
cagctcatgcaacatcatctgctcccaccgtaactctagtacagctgcccaatgggcagacagttcaagtccatggagtcatt
caggcggcccagccatcagttattcagtctccacaagtccaaacagttcagtcttcctgtaaggacttaaaaagacttttctcc
ggaacacagatttcaactattgcagaaagtgaagattcacaggagtcagtggatagtgtaactgattcccaaaagcgaaggg
aaattctttcaaggaggccttcctacaggaaaattttgaatgacttatcttctgatgcaccaggagtgccaaggattgaagaag
agaagtctgaagaggagacttcagcacctgccatcaccactgtaacggtgccaactccaatttaccaaactagcagtggaca
gtatattgccattacccagggaggagcaatacagctggctaacaatggtaccgatggggtacagggcctgcaaacattaac
catgaccaatgcagcagccactcagccgggtactaccattctacagtatgcacagaccactgatggacagcagatcttagtg
cccagcaaccaagttgttgttcaagctgcctctggagacgtacaaacataccagattcgcacagcacccactagcactattgc
ccctggagttgttatggcatcctccccagcacttcctacacagcctgctgaagaagcagcacgaaagagagaggtccgtct
aatgaagaacagggaagcagctcgagagtgtcgtagaaagaagaaagaatatgtgaaatgtttagaaaacagagtggcag
tgcttgaaaatcaaaacaagacattgattgaggagctaaaagcacttaaggacctttactgccacaaatcagatta;
NM004379; NP004370; NP604391.
(SEQ ID NO: 96)
ZFp488: NM001013777:
atggctgagggcaaaggggctcctctgaggccttcagttgagaagagatggaagctcatggaacccaagcagacccagg
cagggatgttcaagaaaatgagccttgtggactctgacactgctgcaggaaagggtagccaagatgaggcctatactgaac
tgagcctgccaacagcaccgaacaagcctcgactggacaggcctcgggcctgcaaggcatacacagagcagaggcaca
ataccttcacagagctatcatgtctccaggagaggccaggggacatccaggcccagacgaggaagctggagaacccaga
aggccagctcggccctcagcagctgccctcgagtttcctcagagcctcaggtgatggcacagtgtgttcagcatggccagg
tgccccccggagtgagcagaaaagtgctttcagcaagccagccaaacgcccagcagagaaacctaagcgctctcccatg
cttctggctggtggaagtgcagagggctcatgggagctctcaggactcatcaccactgtggacatcccatattgggctcatct
gtcaactttcaagttcatgggtgatttctggaaattgcacacattgtcacagaacattctcctctgcaatgctttccagggggctc
ccacaccatggctggagcatacccaggtacaagcccccacatcctcagctccttcctccacagcctcccgggctctcttgcc
gcccacactctcctccttgggcttgtctactcagaactggtgtgcgaagtgcaacctagcctttcgcctgacagctgacctggt
cttccacatgcggtcacatcacaaaagggaacacgtgggccctgacccacattctaagaaacgaagagaggaagttctcac
ttgccccgtttgccacgagtacttccgggagcgccaccatctgtccaggcatatggcttcacatagttag; BC089025;
XM_224697; XP224697.
(SEQ ID NO: 97)
Foxa2: NM_021784: atgctgggagcggtgaagatggaagggcacgagccgtccgactggagcagctactatgca
gagcccgagggctactcctccgtgagcaacatgaacgccggcctggggatgaacggcatgaacacgtacatgagcatgt
cggcggccgccatgggcagcggctcgggcaacatgagcgcgggctccatgaacatgtcgtcgtacgtgggcgctggcat
gagcccgtccctggcggggatgtcccccggcgcgggcgccatggcgggcatgggcggctcggccggggcggccggc
gtggcgggcatggggccgcacttgagtcccagcctgagcccgctcggggggcaggcggccggggccatgggcggcct
ggccccctacgccaacatgaactccatgagccccatgtacgggcaggcgggcctgagccgcgcccgcgaccccaagac
ctacaggcgcagctacacgcacgcaaagccgccctactcgtacatctcgctcatcaccatggccatccagcagagcccca
acaagatgctgacgctgagcgagatctaccagtggatcatggacctcttccccttctaccggcagaaccagcagcgctggc
agaactccatccgccactcgctctccttcaacgactgtttcctgaaggtgccccgctcgcccgacaagcccggcaagggct
ccttctggaccctgcaccctgactcgggcaacatgttcgagaacggctgctacctgcgccgccagaagcgcttcaagtgcg
agaagcagctggcgctgaaggaggccgcaggcgccgccggcagcggcaagaaggcggccgccggagcccaggcct
cacaggctcaactcggggaggccgccgggccggcctccgagactccggcgggcaccgagtcgcctcactcgagcgcct
ccccgtgccaggagcacaagcgagggggcctgggagagctgaaggggacgccggctgcggcgctgagccccccaga
gccggcgccctctcccgggcagcagcagcaggccgcggcccacctgctgggcccgccccaccacccgggcctgccgc
ctgaggcccacctgaagccggaacaccactacgccttcaaccacccgttctccatcaacaacctcatgtcctcggagcagc
agcaccaccacagccaccaccaccaccaaccccacaaaatggacctcaaggcctacgaacaggtgatgcactaccccgg
ctacggttcccccatgcctggcagcttggccatgggcccggtcacgaacaaaacgggcctggacgcctcgcccctggcc
gcagatacctcctactaccagggggtgtactcccggcccattatgaactcctcttaa; NP_068556; NM_012743;
NP036875; NMO10446; NP034576.
Rnx
(SEQ ID NO: 98)
REN: NM_000537: atggatggatggagaaggatgcctcgctggggactgctgctgctgctctggggctcctgtacct
ttggtctcccgacagacaccaccacctttaaacggatcttcctcaagagaatgccctcaatccgagaaagcctgaaggaacg
aggtgtggacatggccaggcttggtcccgagtggagccaacccatgaagaggctgacacttggcaacaccacctcctccg
tgatcctcaccaactacatggacacccagtactatggcgagattgggatcgggaccccaccccaaaccttcaaagtcgtcttt
gacactggttcgtccaatgtttgggtgccctcctccaagtgcagccgtctctacactgcctgtgtgtatcacaagctcttcgatg
cttcggattcctccagctacaagcacaatggaacagaactcaccctccgctattcaacagggacagtcagtggctttctcagc
caggacatcatcaccgtgggtggaatcacggtgacacagatgtttggagaggtcacggagatgcccgccttacccttcatg
ctggccgagtttgatggggttgtgggcatgggcttcattgaacaggccattggcagggtcacccctatcttcgacaacatcat
ctcccaaggggtgctaaaagaggacgtcttctctttctactacaacagagattccgagaattcccaatcgctgggaggacag
attgtgctgggaggcagcgacccccagcattacgaagggaatttccactatatcaacctcatcaagactggtgtctggcaga
ttcaaatgaagggggtgtctgtggggtcatccaccttgctctgtgaagacggctgcctggcattggtagacaccggtgcatc
ctacatctcaggttctaccagctccatagagaagctcatggaggccttgggagccaagaagaggctgtttgattatgtcgtga
agtgtaacgagggccctacactccccgacatctattccacctgggaggcaaagaatacacgctcaccagcgcggactatg
tatttcaggaatcctacagtagtaaaaagctgtgcacactggccatccacgccatggatatcccgccacccactggacccac
ctgggccctgggggccaccttcatccgaaagttctacacagagtttgatcggcgtaacaaccgcattggcttcgccttggcc
cgctga;
(SEQ ID NO: 99)
dL1AND(HAND2): NM_021973:
atgagtctggtaggtggttttccccaccacccggtggtgcaccacgagggctacccgtttgccgccgccgccgccgcagct
gccgccgccgccgccagccgctgcagccatgaggagaacccctacttccatggctggctcatcggccaccccgagatgt
cgccccccgactacagcatggccctgtcctacagccccgagtatgccagcggcgccgccggcctggaccactcccattac
gggggggtgccgccgggcgccgggcccccgggcctgggggggccgcgcccggtgaagcgccgaggcaccgccaa
ccgcaaggagcggcgcaggactcagagcatcaacagcgccttcgccgaactgcgcgagtgcatccccaacgtacccgc
cgacaccaaactctccaaaatcaagaccctgcgcctggccaccagctacatcgcctacctcatggacctgctggccaagga
cgaccagaatggcgaggcggaggccttcaaggcagagatcaagaagaccgacgtgaaagaggagaagaggaagaag
gagctgaacgaaatcttgaaaagcacagtgagcagcaacgacaagaaaaccaaaggccggacgggctggccgcagca
cgtctgggccctggagctcaagcagtga; NMO10402; aspartoacylase (Canavan disease)(ASPA):
NM_000049;
(SEQ ID NO: 100)
atgacttcttgtcacattgctgaagaacatatacaaaaggttgctatctttggaggaacccatgggaatgagctaaccggagta
tltctggttaagcattggctagagaatggcgctgagattcagagaacagggctggaggtaaaaccatttattactaaccccag
agcagtgaagaagtgtaccagatatattgactgtgacctgaatcgcatttttgaccttgaaaatcttggcaaaaaaatgtcaga
agatttgccatatgaagtgagaagggctcaagaaataaatcatttatttggtccaaaagacagtgaagattcctatgacattattt
ttgaccttcacaacaccacctctaacatggggtgcactcttattcttgaggattccaggaataactttttaattcagatgtttcatta
cattaagacttctctggctccactaccctgctacgtttatctgattgagcatccttccctcaaatatgcgaccactcgttccatagc
caagtatcctgtgggtatagaagttggtcctcagcctcaaggggttctgagagctgatatcttggatcaaatgagaaaaatgat
taaacatgctcttgattttatacatcatttcaatgaaggaaaagaatttcctccctgcgccattgaggtctataaaattatagagaa
agttgattacccccgggatgaaaatggagaaattgctgctatcatccatcctaatctgcaggatcaagactggaaaccactgc
atcctggggatcccatgtttttaactcttgatgggaagacgatcccactgggcggagactgtaccgtgtaccccgtgtttgtga
atgaggccgcatattacgaaaagaaagaagcttttgcaaagacaactaaactaacgctcaatgcaaaaagtattcgctgctgt
ttacattag; NM 023113.
(SEQ ID NO: 101)
hexosaminidaseA(HEXA): NM_000520: atgacaagctccaggctttggttttcgctgctgctggcggcagc
gttcgcaggacgggcgacggccctctggccctggcctcagaacttccaaacctccgaccagcgctacgtcctttacccgaa
caactttcaattccagtacgatgtcagctcggccgcgcagcccggctgctcagtcctcgacgaggccttccagcgctatcgt
gacctgcttttcggttccgggtcttggccccgtccttacctcacagggaaacggcatacactggagaagaatgtgttggttgtc
tctgtagtcacacctggatgtaaccagcttcctactttggagtcagtggagaattataccctgaccataaatgatgaccagtgtt
tactcctctctgagactgtctggggagctctccgaggtctggagacttttagccagcttgtttggaaatctgctgagggcacatt
ctttatcaacaagactgagattgaggactttccccgctttcctcaccggggcttgctgttggatacatctcgccattacctgcca
ctctctagcatcctggacactctggatgtcatggcgtacaataaattgaacgtgttccactggcatctggtagatgatccttcctt
cccatatgagagcttcacttttccagagctcatgagaaaggggtcctacaaccctgtcacccacatctacacagcacaggat
gtgaaggaggtcattgaatacgcacggctccggggtatccgtgtgcttgcagagtttgacactcctggccacactttgtcctg
gggaccaggtatccctggattactgactccttgctactctgggtctgagccctctggcacctttggaccagtgaatcccagtct
caataatacctatgagttcatgagcacattcttcttagaagtcagctctgtcttcccagatttttatcttcatcttggaggagatgag
gttgatttcacctgctggaagtccaacccagagatccaggactttatgaggaagaaaggcttcggtgaggacttcaagcagc
tggagtccttctacatccagacgctgctggacatcgtctcttcttatggcaagggctatgtggtgtggcaggaggtgtttgata
ataaagtaaagattcagccagacacaatcatacaggtgtggcgagaggatattccagtgaactatatgaaggagctggaact
ggtcaccaaggccggcttccgggcccttctctctgccccctggtacctgaaccgtatatcctatggccctgactggaaggatt
tctacgtagtggaacccctggcatttgaaggtacccctgagcagaaggctctggtgattggtggagaggcttgtatgtgggg
agaatatgtggacaacacaaacctggtccccaggctctggcccagagcaggggctgttgccgaaaggctgtggagcaac
aagttgacatctgacctgacatttgcctatgaacgtttgtcacacttccgctgtgagttgctgaggcgaggtgtccaggcccaa
cccctcaatgtaggcttctgtgagcaggagtttgaacagacctga.
(SEQ ID NO: 102)
Lesch_Nyhan_syndromc(HRPT): NM_000194: atggcgacccgcagccctggcgtcgtgattagtgatg
atgaaccaggttatgaccttgatttattttgcatacctaatcattatgctgaggatttggaaagggtgtttattcctcatggactaat
tatggacaggactgaacgtcttgctcgagatgtgatgaaggagatgggaggccatcacattgtagccctctgtgtgctcaag
gggggctataaattctttgctgacctgctggattacatcaaagcactgaatagaaatagtgatagatccattcctatgactgtag
attttatcagactgaagagctattgtaatgaccagtcaacaggggacataaaagtaattggtggagatgatctctcaactttaac
tggaaagaatgtcttgattgtggaagatataattgacactggcaaaacaatgcagactttgctttccttggtcaggcagtataat
ccaaagatggtcaaggtcgcaagcttgctggtgaaaaggaccccacgaagtgttggatataagccagactttgttggatttg
aaattccagacaagtttgttgtaggatatgcccttgactataatgaatacttcagggatttgaatcatgtttgtgtcattagtgaaa
ctggaaaagcaaaatacaaagcctaa; NM_204848.
Huntingtin; NM_10414;
(SEQ ID NO: 103)
GUSB; NM_000181: atggcccgggggtcggcggttgcctgggcggcgctcgggccgttgttgtggggctgcgcg
ctggggctgcagggcgggatgctgtacccccaggagagcccgtcgcgggagtgcaaggagctggacggcctctggag
cttccgcgccgacttctctgacaaccgacgccggggcttcgaggagcagtggtaccggcggccgctgtgggagtcaggc
cccaccgtggacatgccagttccctccagcttcaatgacatcagccaggactggcgtctgcggcattttgtcggctgggtgt
ggtacgaacgggaggtgatcctgccggagcgatggacccaggacctgcgcacaagagtggtgctgaggattggcagtg
cccattcctatgccatcgtgtgggtgaatggggtcgacacgctagagcatgaggggggctacctccccttcgaggccgaca
tcagcaacctggtccaggtggggcccctgccctcccggctccgaatcactatcgccatcaacaacacactcacccccacca
ccctgccaccagggaccatccaatacctgactgacacctccaagtatcccaagggttactttgtccagaacacatattttgact
ttttcaactacgctggactgcagcggtctgtacttctgtacacgacacccaccacctacatcgatgacatcaccgtcaccacca
gcgtggagcaagacagtgggctggtgaattaccagatctctgtcaagggcagtaacctgttcaagttggaagtgcgtcttttg
gatgcagaaaacaaagtcgtggcgaatgggactgggacccagggccaacttaaggtgccaggtgtcagcctctggtggc
cgtacctgatgcacgaacgccctgcctatctgtattcattggaggtgcagctgactgcacagacgtcactggggcctgtgtct
gacttctacacactccctgtggggatccgcactgtggctgtcaccaagagccagttcctcatcaatgggaaacctttctatttc
cacggtgtcaacaagcatgaggatgcggacatccgagggaagggcttcgactggccgctgctggtgaaggacttcaacct
gcttcgctggcttggtgccaacgctttccgtaccagccactacccctatgcagaggaagtgatgcagatgtgtgaccgctatg
ggattgtggtcatcgatgagtgtcccggcgtgggcctggcgctgccgcagttcttcaacaacgtttctctgcatcaccacatg
caggtgatggaagaagtggtgcgtagggacaagaaccaccccgcggtcgtgatgtggtctgtggccaacgagcctgcgt
cccacctagaatctgctggctactacttgaagatggtgatcgctcacaccaaatccttggacccctcccggcctgtgacctttg
tgagcaactctaactatgcagcagacaagggggctccgtatgtggatgtgatctgtttgaacagctactactcttggtatcacg
actacgggcacctggagttgattcagctgcagctggccacccagtttgagaactggtataagaagtatcagaagcccattatt
cagagcgagtatggagcagaaacgattgcagggtttcaccaggatccacctctgatgttcactgaagagtaccagaaaagt
ctgctagagcagtaccatctgggtctggatcaaaaacgcagaaaatacgtggttggagagctcatttggaattttgccgatttc
atgactgaacagtcaccgacgagagtgctggggaataaaaaggggatcttcactcggcagagacaaccaaaaagtgcag
cgttccttttgcgagagagatactggaagattgccaatgaaaccaggtatccccactcagtagccaagtcacaatgtttggaa
aacagcccgtttacttga; NM_010368.
NPC1: NMJ)00271;NM_006432.
(SEQ ID NO: 104)
hexosaminidaseB: NM_000521; atggagctgtgcgggctggggctgccccggccgcccatgctgctggcgct
gctgttggcgacactgctggcggcgatgttggcgctgctgactcaggtggcgctggtggtgcaggtggcggaggcggctc
gggccccgagcgtctcggccaagccggggccggcgctgtggcccctgccgctctcggtgaagatgaccccgaacctgct
gcatctcgccccggagaacttctacatcagccacagccccaattccacggcgggcccctcctgcaccctgctggaggaag
cgtttcgacgatatcatggctatatttttggtttctacaagtggcatcatgaacctgctgaattccaggctaaaacccaggttcag
caacttcttgtctcaatcacccttcagtcagagtgtgatgctttccccaacatatcttcagatgagtcttatactttacttgtgaaag
aaccagtggctgtccttaaggccaacagagtttggggagcattacgaggtttagagacctttagccagttagtttatcaagatt
cttatggaactttcaccatcaatgaatccaccattattgattctccaaggttttctcacagaggaattttgattgatacatccagac
attatctgccagttaagattattcttaaaactctggatgccatggcttttaataagtttaatgttcttcactggcacatagttgatgac
cagtctttcccatatcagagcatcacttttcctgagttaagcaataaaggaagctattctttgtctcatgtttatacaccaaatgatg
tccgtatggtgattgaatatgccagattacgaggaattcgagtcctgccagaatttgatacccctgggcatacactatcttggg
gaaaaggtcagaaagacctcctgactccatgttacagtagacaaaacaagttggactcttttggacctataaaccctactctga
atacaacatacagcttccttactacatttttcaaagaaattagtgaggtgtttccagatcaattcattcatttgggaggagatgaa
gtggaatttaaatgttgggaatcaaatccaaaaattcaagatttcatgaggcaaaaaggctttggcacagattttaagaaacta
gaatctttctacattcaaaaggttttggatattattgcaaccataaacaagggatccattgtctggcaggaggtttttgatgataaa
gcaaagcttgcgccgggcacaatagttgaagtatggaaagacagcgcatatcctgaggaactcagtagagtcacagcatct
ggcttccctgtaatcctttctgctccttggtacttagatttgattagctatggacaagattggaggaaatactataaagtggaacc
tcttgattttggcggtactcagaaacagaaacaacttttcattggtggagaagcttgtctatggggagaatatgtggatgcaact
aacctcactccaagattatggcctcgggcaagtgctgttggtgagagactctggagttccaaagatgtcagagatatggatg
acgcctatgacagactgacaaggcaccgctgcaggatggtcgaacgtggaatagctgcacaacctctttatgctggatattg
taaccatgagaacatgtaa.
(SEQ ID NO: 105)
galactosidase, alpha(GLA): NM000169:
atgcagctgaggaacccagaactacatctgggctgcgcgcttgcgcttcgcttcctggccctcgtttcctgggacatccctgg
ggctagagcactggacaatggattggcaaggacgcctaccatgggctggctgcactgggagcgcttcatgtgcaaccttga
ctgccaggaagagccagattcctgcatcagtgagaagctcttcatggagatggcagagctcatggtctcagaaggctggaa
ggatgcaggttatgagtacctctgcattgatgactgttggatggctccccaaagagattcagaaggcagacttcaggcagac
cctcagcgctttcctcatgggattcgccagctagctaattatgttcacagcaaaggactgaagctagggatttatgcagatgtt
ggaaataaaacctgcgcaggcttccctgggagttttggatactacgacattgatgcccagacctttgctgactggggagtag
atctgctaaaatttgatggttgttactgtgacagtttggaaaatttggcagatggttataagcacatgtccttggccctgaatagg
actggcagaagcattgtgtactcctgtgagtggcctctttatatgtggccctttcaaaagcccaattatacagaaatccgacagt
actgcaatcactggcgaaattttgctgacattgatgattcctggaaaagtataaagagtatcttggactggacatcttttaacca
ggagagaattgttgatgttgctggaccagggggttggaatgacccagatatgttagtgattggcaactttggcctcagctgga
atcagcaagtaactcagatggccctctgggctatcatggctgctcctttattcatgtctaatgacctccgacacatcagccctca
agccaaagctctccttcaggataaggacgtaattgccatcaatcaggaccccttgggcaagcaagggtaccagcttagaca
gggagacaactttgaagtgtgggaacgacctctctcaggcttagcctgggctgtagctatgataaaceggcaggagattggt
ggacctcgctcttataccatcgcagttgcttccctgggtaaaggagtggcctgtaatcctgcctgcttcatcacacagctcctc
cctgtgaaaaggaagctagggttctatgaatggacttcaaggttaagaagtcacataaatcccacaggcactgttttgcttcag
ctagaaaatacaatgcagatgtcattaaaagacttactttaa
(SEQ ID NO: 106)
glucosidasE beta_acid(GBA): NM_000157:
atggagttttcaagtccttccagagaggaatgtcccaagcctttgagtagggtaagcatcatggctggcagcctcacaggatt
gcttctacttcaggcagtgtcgtgggcatcaggtgcccgcccctgcatccctaaaagcttcggctacagctcggtggtgtgtg
tctgcaatgccacatactgtgactcctttgaccccccgacctttcctgcccttggtaccttcagccgctatgagagtacacgca
gtgggcgacggatggagctgagtatggggcccatccaggctaatcacacgggcacaggcctgctactgaccctgcagcc
agaacagaagttccagaaagtgaagggatttggaggggccatgacagatgctgctgctctcaacatccttgccctgtcaccc
cctgcccaaaatttgctacttaaatcgtacttctctgaagaaggaatcggatataacatcatccgggtacccatggccagctgt
gacttctccatccgcacctacacctatgcagacacccctgatgatttccagttgcacaacttcagcctcccagaggaagatac
caagctcaagatacccctgattcaccgagccctgcagttggcccagcgtcccgtttcactccttgccagcccctggacatca
cccacttggctcaagaccaatggagcggtgaatgggaaggggtcactcaagggacagcccggagacatctaccaccaga
cctgggccagatactttgtgaagttcctggatgcctatgctgagcacaagttacagttctgggcagtgacagctgaaaatgag
ccttctgctgggctgttgagtggataccccttccagtgcctgggcttcacccctgaacatcagcgagacttcattgcccgtgac
ctaggtcctaccctcgccaacagtactcaccacaatgtccgcctactcatgctggatgaccaacgcttgctgctgccccactg
ggcaaaggtggtactgacagacccagaagcagctaaatatgttcatggcattgctgtacattggtacctggactttctggctc
cagccaaagccaccctaggggagacacaccgcctgttccccaacaccatgctctttgcctcagaggcctgtgtgggctcca
agttctgggagcagagtgtgcggctaggctcctgggatcgagggatgcagtacagccacagcatcatcacgaacctcctgt
accatgtggtcggctggaccgactggaaccttgccctgaaccccgaaggaggacccaattgggtgcgtaactttgtcgaca
gtcccatcattgtagacatcaccaaggacacgttttacaaacagcccatgttctaccaccttggccacttcagcaagttcattcc
tgagggctcccagagagtggggctggttgccagtcagaagaacgacctggacgcagtggcactgatgcatcccgatggct
ctgctgttgtggtcgtgctaaaccgctcctctaaggatgtgcctcttaccatcaaggatcctgctgtgggcttcctggagacaat
ctcacctggctactccattcacacctacctgtggcgtcgccagtga; NM_008094.
(SEQ ID NO: 107)
von_Hippel_Lindau_tumor_suppressor(VHL): NM_000551:
atgccccggagggcggagaactgggacgaggccgaggtaggcgcggaggaggcaggcgtcgaagagtacggccctg
aagaagacggcggggaggagtcgggcgccgaggagtccggcccggaagagtccggcccggaggaactgggcgccg
aggaggagatggaggccgggcggccgcggcccgtgctgcgctcggtgaactcgcgcgagccctcccaggtcatcttctg
caatcgcagtccgcgcgtcgtgctgcccgtatggctcaacttcgacggcgagccgcagccctacccaacgctgccgcctg
gcacgggccgccgcatccacagctaccgaggtcacctttggctcttcagagatgcagggacacacgatgggcttctggtta
accaaactgaattatttgtgccatctctcaatgttgacggacagcctatttttgccaatatcacactgccagtgtatactctgaaa
gagcgatgcctccaggttgtccggagcctagtcaagcctgagaattacaggagactggacatcgtcaggtcgctctacgaa
gatctggaagaccacccaaatgtgcagaaagacctggagcggctgacacaggagcgcattgcacatcaacggatgggag
attga.
(SEQ ID NO: 108)
Beta_globin(HBB): NM_000518: atggtgcatctgactcctgaggagaagtctgccgttactgccctgtggggc
aaggtgaacgtggatgaagttggtggtgaggccctgggcaggctgctggtggtctacccttggacccagaggttctttgagt
cctttggggatctgtccactcctgatgctgttatgggcaaccctaaggtgaaggctcatggcaagaaagtgctcggtgccttta
gtgatggcctggctcacctggacaacctcaagggcacctttgccacactgagtgagctgcactgtgacaagctgcacgtgg
atcctgagaacttcaggctcctgggcaacgtgctggtctgtgtgctggcccatcactttggcaaagaattcaccccaccagtg
caggctgcctatcagaaagtggtggctggtgtggctaatgccctggcccacaagtatcactaa.
(SEQ ID NO: 109)
PARK2: NM_013988: atgatagtgtttgtcaggttcaactccagccatggtttcccagtggaggtcgattctgacacc
agcatcttccagctcaaggaggtggttgctaagcgacagggggttccggctgaccagttgcgtgtgattttcgcagggaag
gagctgaggaatgactggactgtgcaggaatttttctttaaatgtggagcacaccccacctctgacaaggaaacatcagtag
ctttgcacctgatcgcaacaaatagtcggaacatcacttgcattacgtgcacagacgtcaggagccccgtcctggttttccagt
gcaactcccgccacgtgatttgcttagactgtttccacttatactgtgtgacaagactcaatgatcggcagtttgttcacgaccct
caacttggctactccctgccttgtgtggctggctgtcccaactccttgattaaagagctccatcacttcaggattctgggagaa
gagcagtacaaccggtaccagcagtatggtgcagaggagtgtgtcctgcagatggggggcgtgttatgcccccgccctgg
ctgtggagcggggctgctgccggagcctgaccagaggaaagtcacctgcgaagggggcaatggcctgggctgtgggttt
gccttctgccgggaatgtaaagaagcgtaccatgaaggggagtgcagtgccgtatttgaagcctcaggaacaactactcag
gcctacagagtcgatgaaagagccgccgagcaggctcgttgggaagcagcctccaaagaaaccatcaagaaaaccacca
agccctgtccccgctgccatgtaccagtggaaaaaaatggaggctgcatgcacatgaagtgtccgcagccccagtgcagg
ctcgagtggtgctggaactgtggctgcgagtggaaccgcgtctgcatgggggaccactggttcgacgtgtag;
NM 004562; NM 020093.

The contents of all parenthetically cited publications and the following United States Patents, are noted and incorporated by reference in their entireties: U.S. Pat. Nos. 7,211,247, 5,677,139, 6,432,711 and 5,453,357, 5,593,875, 5,783,566, 5,928,944, 5,910,488, 5,824,547.

What is claimed are:

1. A cell culture dish made of non media-permeable material and having a base and a plurality of separate juxtaposed side-by-side wells having common interior well walls preventing physical contact or movement of chemical factors between the separated cell or tissue cultures, the walls having different heights and defining an outer “surround” cell or tissue culture and one or more inner “center” cell or tissue cultures to enable contained cell or tissue communication between the well spaces, the wells configured to comprise two or more cell or tissue cultures, the wells permitting signal communication between the cells or tissues situated within said wells, and further comprising wall material, electrodes and/or electrode contacts, and well dimensions suitable for facilitating electroporation.

2. The method inducing cellular behavior displayed in a first cell population that is displayed in a second cell population wherein the method comprises culturing the cells in the cell culture dish of claim 1.

3. The method of claim 2 where the desired induced behavior comprises one or more changes in signal transduction.

4. The method of claim 2 where the desired induced behavior comprises one or more changes in ion current flows.

5. The method of claim 2 where the desired induced behavior comprises a change in gene expression.

6. The method of claim 2 where the desired induced behavior comprises a change in protein expression.

7. The method of claim 2 where the desired induced behavior comprises a change in cellular markers.

8. The method of claim 2 where the desired induced behavior comprises a change in cellular phenotype.

9. The method of claim 2 where the desired induced behavior comprises a change in antibody reactivity.

10. The method of claim 2 where the desired induced behavior comprises a change allowing differential cell sorting.

11. The method of claim 2 where the desired induced behavior comprises a change in potency.

12. The method of claim 2 wherein cells of the first cell population are induced to greater potency.

13. The method of claim 2 wherein cells of the first cell population are induced to a new, differentiated cell phenotype.

14. The method of claim 2 wherein cells of the first cell population are further cultured in a 2 Dimensional or 3 Dimensional format.

15. The method of claim 2 wherein cells of the first cell population are genetically-modified.

16. The method of claim 2 wherein cells of the first cell population are contacted with agents further promoting the desired induced behavior.

17. The method of claim 2 wherein the first and/or second cell population is a somatic cell population.

18. The method of claim 2 wherein the first and/or second cell population is stem cell population.

19. The method of claim 2. wherein the first and/or second cell population displays multipotent, pluripotent or totipotent stem cell behavior.

20. The method of claim 2 wherein the medium comprises agents promoting cellular differentiation.

21. The method of claim 2 wherein the medium comprises nucleic acids or proteins.

22. The method of claim 2 wherein the medium comprises non-nucleic acids and non-proteins.

23. The method of claim 21 wherein cells of the first cell population are contacted with nucleic acid or protein transcription factors or other cell fate determinants.

24. The cells, vectors, agents, proteins, nucleic acids, transcription factors, and other cell fate determinants of claims 1-23.

25. The method of treatment comprising administering to a subject, the cells, agents, nucleic acids or proteins of claims 1-24.
